(19) United States
(12) Patent Application Publication
(10) Pub. No.: US 2022/0241331 A1
(43) Pub. Date: Aug. 4, 2022

(54) IDENTIFICATION OF RECURRENT MUTATED NEOPEPTIDES

(71) Applicant: Yeda Research and Development Co. Ltd., Rehovot (IL)

(72) Inventors: Yardena SAMUELS, Rehovot (IL); Shelly KALAORA, Rehovot (IL); Aviyah PERI, Rehovot (IL); Nir FRIEDMAN, Rehovot (IL); Shlomit REICH-ZELIGER, Rehovot (IL); Erez GREENSTEIN, Rehovot (IL)

(73) Assignee: Yeda Research and Development Co. Ltd., Rehovot (IL)

(21) Appl. No.: 17/612,279

(22) PCT Filed: May 19, 2020
(86) PCT No.: PCT/IL2020/050547

§ 371 Date: Nov. 18, 2021

(30) Foreign Application Priority Data
May 19, 2019 (IL) 266728

(51) Int. Cl. A61K 35/17; C07K 14/47; C07K 14/725; C07K 14/82; C12N 5/0783

(52) U.S. Cl. CPC A61K 35/17; C07K 14/4748; C12N 2510/00; C07K 14/82; C12N 5/0636; C07K 14/7051

(57) ABSTRACT

A method of treating cancer in a subject is disclosed. The method comprises administering to the subject a therapeutically effective amount of T cells expressing a T cell receptor (TCR) having a CDR3 amino acid sequence selected from the group consisting of 199-210.

### RELATED APPLICATIONS

This application claims the benefit of priority of Israeli Patent Application No. 266728 filed 19 May 2019, the contents of which are incorporated herein by reference in their entirety.

### SEQUENCE LISTING STATEMENT

The ASCII file, entitled 80448 Sequence Listing.txt, created on 19 May 2020, comprising 97,674 bytes, submitted concurrently with the filing of this application is incorporated herein by reference.

### FIELD AND BACKGROUND OF THE INVENTION

The present invention, in some embodiments thereof, relates to T cell receptors which bind to recurrent mutated neopeptides and method of identifying the recurrent mutated neopeptides.

Immunotherapy sparked new hope for oncology in recent years, due to its remarkable ability to induce long-term tumor regression of metastatic cancer. This feature is shared across immunotherapeutic modalities, including both checkpoint blockade and adoptive cell transfer (ACT) of TILs. It is believed that the final common pathway of these two treatments is specific recognition of tumor antigens by cytotoxic T-lymphocytes. Specifically, with the advancement of sequencing capabilities, the in-depth dissection of immunotherapy success stories has revealed a center-stage role for mutation-derived antigens, designated neo-antigens, in mediating an anti-tumor immune response.

Neo-antigens are cell-surface peptide/human-leukocyte antigen (HLA) complexes where the peptide component, i.e., the neo-peptide, is the altered degradation product of a mutated protein. Restricted in expression to the diseased tissue, and uncurbed by immune tolerance, neo-antigens may elicit specific anti-tumor reactivity upon TCR engagement, and are therefore ideal therapeutic targets.

The great majority of neo-antigens identified from treated patients derive from private, non-recurring, mutations, and thus, although effective, cannot be generalized beyond the individual patient. Hotspot neo-antigens, i.e., neo-antigens that appear in a large group of cancer patients, clearly form only at the intersection of recurrent oncogenic mutations and common HLA alleles. Such neo-antigens are highly sought after for two main reasons. First, hotspot neo-antigens may pave the way toward “off-the-shelf” cellular treatments, vaccines and patient screening strategies. Tumor cells expressing validated mutation/HLA combinations should be amenable to immunotherapy. Even in the absence of a priori immune recognition, pre-determined TCRs, from other patients or even healthy donors, can be used to redirect autologous T-cells against neglected hotspot neo-antigens. Moreover, neo-antigen-specific T-cells, undetectable prior to treatment, have been shown to expand significantly following mutation-based vaccines. Second, hotspot neo-antigens are potentially superior to private neo-antigens as treatment targets. This is because immunotherapy directed at sub-clonal mutations of heterogeneous tumors might give way to immune escape, whereas hotspot neo-antigens, which are derived from clonal oncogenic mutations, are expected to present more homogenously within tumors.

The several hotspot neo-antigens uncovered over the years stem from major oncogenes, such as BRAF, NRAS and p53. The clinical relevance of such neo-antigens, however, was directly demonstrated through successful ACT treatment of a patient with metastatic colon cancer, utilizing autologous TILs directed at a newly discovered HLA-C*08:02/KRAS.G12D hotspot neo-antigen. These recent successes propelled the reemergence of endeavors to discover hotspot neo-antigens. A p53-centered screen revealed native TIL reactivity toward derived neo-antigens in 8% of screened patients, with hotspot neo-antigens presenting in several cases. Other efforts focused on the identification of T-cells from the peripheral blood of patients or healthy donors that target specific hotspots, thereby expanding the repertoire of known KRAS and other oncogene-derived neo-antigens for both HLA class-I and HLA class-II.

To date, neo-antigen discovery efforts are almost exclusively T-cell centric. In these methods, candidate neo-peptides are artificially expressed in antigen presenting cells (APCs), either as pulsed synthetic peptides or via minigene overexpression. APCs are then co-incubated with T-cells, most commonly TILs, and their response profile interpreted for indirect identification of neo-antigens. Further characterization and validation rely heavily on in silico binding predictions, such that identified neo-antigens are restricted to those that are both predicted to bind and are immunogenic in the tested patient. Moreover, irrelevant neo-antigens, which were edited out from the presented repertoire in tumor evolution, will nonetheless be identified as long as they were once immunogenic.

The causal role of RAS proteins in cancer has long been recognized, with activating mutations appearing in a third of all human cancers²⁸. The three main isoforms, KRAS, NRAS and HRAS, share an identical, 86-amino-acid-long N-termini. Within this identical stretch, three mutational hotspots were recognized: at positions 12, 13 and 61. Pan-cancer, KRAS is the most highly mutated RAS isoform (85% of RAS mutations). However, in melanoma, the most successful immunotherapy target to date, NRAS mutations dominate. Specifically, NRAS.61 is the second most highly mutated position in melanoma, appearing in as many as 20% of patients. NRAS-mutant melanoma is associated with poorer outcomes, compared with non-NRAS-mutant melanoma. The multiple attempts to develop RAS-targeted therapy have yet to yield effective, specifically approved therapies for NRAS-mutant melanoma.

### SUMMARY OF THE INVENTION

According to an aspect of the present invention there is provided a method of treating cancer in a subject comprising administering to the subject a therapeutically effective amount of T cells expressing a T cell receptor (TCR) having a CDR3 amino acid sequence selected from the group consisting of 199-210, thereby treating the cancer of the subject.

According to an aspect of the present invention there is provided an isolated population of T cells genetically modified to express a T cell receptor (TCR) having a CDR3 amino acid sequence selected from the group consisting of 199-210.

According to an aspect of the present invention there is provided a use of the isolated population of T cells disclosed herein for treatment of cancer.

According to an aspect of the present invention there is provided a method of selecting a recurrent HLA-presented neoantigen which can be targeted in a cancer-immunotherapy treatment, the method comprising:
(a) analyzing the frequency of occurrence of a cancer-associated mutated protein in the context of an individual HLA allele in a plurality of cancer patients; and
(b) determining the binding affinity of peptides of 8-14 amino acids in length derived from the cancer-associated mutated protein to the individual HLA allele, wherein the peptides comprise a mutation compared to the wild-type protein, wherein a candidate peptide which binds with an affinity above a first predetermined level to an HLA allele having a frequency of occurrence above a second predetermined level, is selected as an HLA-presented neoantigen that can be targeted in a cancer-immunotherapy treatment.

According to an aspect of the present invention there is provided a method of selecting a subject suffering from cancer for cancer-immunotherapy treatment comprising:

(a) ascertaining the HLA profile of a subject;

(b) determining whether the subject comprises a genome which encodes a cancer-associated mutated protein; wherein the subject is selected for treatment when:

(i) the HLA profile of the subject occurs with a frequency above a predetermined level in a plurality of cancer patients;

(ii) the cancer-associated mutated protein of the subject occurs with a frequency above a predetermined level in a plurality of cancer patients; and

(iii) at least one peptide of 8-14 amino acids in length derived from the cancer-associated mutated protein binds to an HLA which is of the identical allele to the subject above a predetermined level, wherein the peptide comprises a mutation compared to the wild-type protein.

According to an aspect of the present invention there is provided a method of treating a subject suffering from cancer using cancer immunotherapy treatment, the method comprising:

(a) selecting the subject according to claim 24; and

(b) treating the subject with a therapeutically effective amount of an agent that targets the at least one peptide, thereby treating the subject.

According to an aspect of the present invention there is provided a method of treating cancer of a subject comprising:

(a) ascertaining the HLA profile of a subject;

(b) determining whether the subject expresses NRAS.Q61K; and

(c) when the subject has been identified as being HLA-A*01:01/NRAS.Q61K, treating the subject with a therapeutically effective amount of an agent that targets the peptide having an amino acid sequence as set forth in SEQ ID NO: 1, thereby treating the cancer.

According to an aspect of the present invention there is provided a method of treating cancer of a subject comprising:

(a) ascertaining the HLA profile of a subject;

(b) determining whether the subject expresses a RAS variant selected from the group consisting of Q61K, Q61R, Q61L and Q61H; and

(c) when the subject expresses the RAS variant, treating the subject with a therapeutically effective amount of an agent that targets a peptide having an amino acid sequence selected from the group consisting of SEQ ID NOs: 1 and 12-132, wherein the peptide is selected according to the corresponding HLA profile as set forth in Table 1C.

According to an aspect of the present invention there is provided a of an agent that targets the peptide having an amino acid sequence as set forth in SEQ ID NO: 1, for treating cancer in a subject, when the subject has been identified as being HLA-A*01:01/NRAS.Q61K.

According to an aspect of the present invention there is provided a of an agent that targets a peptide having an amino acid sequence selected from the group consisting of SEQ ID NOs: 1 and 12-132 for treating cancer in a subject when the subject expresses a RAS variant selected from the group consisting of Q61K, Q61R, Q61L and Q61H, wherein the peptide is selected according to the corresponding HLA profile as set forth in Table 1C.

According to an embodiment of the present invention, the TCR binds to a peptide having a sequence as set forth in SEQ ID NO: 1 in a complex with HLA-A*01:01 allele in the subject.

According to an embodiment of the present invention, the T cells are autologous to the subject.

According to an embodiment of the present invention, the T cells are non-autologous to the subject.

According to an embodiment of the present invention, the T cells are genetically modified to express the T cell receptor.

According to an embodiment of the present invention, the T cells comprise CD8+ T cells.

According to an embodiment of the present invention, the cancer is selected from the group consisting of melanoma, colon cancer, breast cancer, thyroid cancer, stomach cancer, colorectal cancer, leukemia cancer, bladder cancer, lung cancer, ovarian cancer, breast cancer and prostate cancer.

According to an embodiment of the present invention, the cancer is melanoma.

According to an embodiment of the present invention, the method further comprises treating the subject with a checkpoint inhibitor.

According to an embodiment of the present invention, the isolated population of T cells are CD8+ T cells.

According to an embodiment of the present invention, the determining comprises predicting the binding affinity using a prediction algorithm.

According to an embodiment of the present invention, the prediction algorithm comprises NetMHCpan.

According to an embodiment of the present invention, the method further comprises corroborating that the candidate peptide binds to the HLA allele in at least one cancer patient.

According to an embodiment of the present invention, the HLA comprises HLA class I.

According to an embodiment of the present invention, the HLA class I comprises HLA-A.

According to an embodiment of the present invention, the HLA-A comprises HLA-A*01:01.

According to an embodiment of the present invention, the cancer-associated mutated protein is a member of the RAS family.

According to an embodiment of the present invention, the member is selected from the group consisting of NRAS, KRAS and HRAS.

According to an embodiment of the present invention, the member is NRAS.

According to an embodiment of the present invention, the cancer associated mutated protein is a RAF kinase.

According to an embodiment of the present invention, the RAF kinase is B-RAF.

According to an embodiment of the present invention, the cancer patients comprise melanoma patients, thyroid cancer patients, pheochromocytoma patients, seminoma patients, stomach adenocarcinoma patients, cholangiocarcinoma patients, pancreatic adenocarcinoma patients, colorectal adenocarcinoma, leukemia patients, bladder urothelial carcinoma patients, endometrial carcinoma patients, thymic epithelial tumor patients, non-small cell lung cancer patients, sarcoma patients, ovarian cancer patients and prostate cancer patients.

According to an embodiment of the present invention, the cancer is a metastatic cancer.

According to an embodiment of the present invention, the cancer is selected from the group consisting of melanoma, colon cancer, breast cancer, thyroid cancer, stomach cancer, colorectal cancer, leukemia cancer, bladder cancer, lung cancer, ovarian cancer, breast cancer and prostate cancer.

According to an embodiment of the present invention, the cancer-associated mutated protein is a member of the RAS family.

According to an embodiment of the present invention, the member is selected from the group consisting of NRAS, KRAS and HRAS.

According to an embodiment of the present invention, the member is NRAS.

According to an embodiment of the present invention, the cancer-associated mutated protein is a RAF kinase.

According to an embodiment of the present invention, the RAF kinase is B-RAF.

According to an embodiment of the present invention, the agent is selected from the group consisting of a vaccine, an antibody and a population of T cells expressing a receptor that targets the T cell epitope.

According to an embodiment of the present invention, the peptide has an amino acid sequence selected from the group consisting of SEQ ID NOs: 1 and 12-28.

According to an embodiment of the present invention, the RAS variant is NRAS.

According to an embodiment of the present invention, the cancer is selected from the group consisting of melanoma, colon cancer, breast cancer, thyroid cancer, stomach cancer, colorectal cancer, leukemia cancer, bladder cancer, lung cancer, ovarian cancer, breast cancer and prostate cancer.

According to an embodiment of the present invention, the agent is selected from the group consisting of a vaccine, an antibody and a population of T cells expressing a receptor that targets the T cell epitope.

According to an embodiment of the present invention, the method further comprises treating the subject with a checkpoint inhibitor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates the recurrent neo-antigen discovery pipeline. Mutational status and HLA-allotyping of 6,048 cancer patients, with 364 melanoma patients among them, were combined to infer cancer-relevant high-recurrence RAS.61/HLA-allele combinations. These were intersected with peptide binding predictions, enumerating over all possible RAS.61-derived peptides. HLA-A*01:01/RAS.61 stood out as the most promising candidate, combining strongest prediction scores with high frequency of occurrence. Further analysis thus focused on HLA-A*01:01/RAS.61. Two melanoma/TIL samples in the tumor bank were found to harbor the relevant HLA-A*01:01/NRAS. Q61K combination, focusing further efforts. HLA-peptidomics was applied for direct neo-antigen identification. Discovery mode analysis unbiasedly uncovered HLA-A*01:01/ILDTAGKEEY (SEQ ID NO: 1) as a neo-antigen presented on melanoma cells. Absolute targeted mass spectrometry was utilized to prove robustness of presentation and to quantify the neo-peptide in three additional melanoma samples. Neo-antigen immunogenicity was tested in the two available TIL population, revealing specific reactivity and killing capacity. Tetramer-sorted and reactive TILs were sequenced to identify candidate effective, neo-antigen specific, TCR sequences.

FIGS. 2A-E: Data-driven NRAS neo-peptide/HLA allele candidate selection and presentation validation using HLA-peptidomics.

(A) Frequency of HLA-A*01:01/RAS.61-mutant combination in the TCGA melanoma cohort. Left pie chart: HLA-A*01:01, RAS.61 mutations and combined HLA-A*01:01/RAS.61 combination frequencies in melanoma patients. Right pie chart: isoform/substitution distribution among melanoma patients harboring the HLA-A*01:01/RAS.61-mutant combination. (B) A*01:01/RAS.61 is both prevalent and predicted to yield a neo-antigen. X axis: percent of patients with HLA-allele/RAS.61-mutant combination in TCGA melanoma cohort. Y axis: modified best % Rank of NetMHCpan 4.0 binding prediction; Values are calculated as: (2-% Rank) for % Rank<=2, 0 for % Rank>2. (C) Predicted complex structures for HLA-A*01:01 in complex with RAS peptides ILDTAGQEEY (SEQ ID NO: 2; wild-type) and ILDTAGKEEY (SEQ ID NO: 1; mutant, RAS.Q61K). HLA shown in grey cartoon, peptide backbone represented as ribbons, with P7 residue (position 61) sidechain atoms shown. Hydrogens omitted for clarity. Left panel—Overlaid ILDTAGQEEY (SEQ ID NO: 2) complex structures. Right panel—Overlaid ILDTAGKEEY (SEQ ID NO: 1) complex structures. (D) Tandem mass spectra of the ILDTAGKEEY (SEQ ID NO: 1) neo-peptide as it was identified in HLA-peptidomics of the 17T tumor cell-line, harboring the A*01:01/NRAS.Q61K combination. (E) ILDTAGKEEY (SEQ ID NO: 1) neo-peptide was identified in multiple melanoma samples harboring the HLA-A*01:01/NRAS.Q61K combination using HLA-peptidomics.

FIGS. 3A-D: 17TIL and 135TIL show HLA-A*01:01/ILDTAGKEEY (SEQ ID NO: 1)-specific reactivity and killing capacity. (A) TIL populations 17TIL and 135TIL show neo-epitope specific reactivity. IFNγ release from bulk TIL as measured in ELISA. IHW01161, IHW01113, IHW01070-B-LCL harboring HLA-A*01:01. Supernatants were diluted 1:20 prior to assay performance. Specimens exceeding the highest standard control are shown at the maximal concentration of 1000 pg/ml. (B) IFNγ release measured in ELISA with tittered concentrations of pulsed peptides. Supernatants were diluted 1:40 prior to assay performance. (C) A*01:01/ILDTAGKEEY (SEQ ID NO: 1)-tetramer staining of bulk 17TIL. (D) Tetramer-positive 17TIL is able to kill cognate melanoma in a dose dependent manner. In-vitro killing assay of tetramer sorted 17TIL incubated at tittered E:T ratio with GFP-tagged cognate melanoma. In all panels, error bars represent standard deviation of triplicates, t test was used to evaluate significance.

FIGS. 4A-I: Dissection of neo-antigen specific and reactive T-cell clones in 17TIL and 135TIL. Percentages and log 10 TCR-chain frequencies as obtained from bulk TCR sequencing. (A-D) tetramer-positive (Y axis) vs. tetramer-negative (X axis) sorted TIL subpopulations. Colored dots represent our neo-antigen specific candidates. These are chains that are highly enriched in the tetramer-positive TCR repertoire and consist at least 1% of it. (A) TCRα 17TIL; (B) TCRβ 17TIL; (C) TCRα 135TIL; (D) TCRβ 135TIL. (E-H) 4-1BB positive (Y axis) vs. CD4 negative (X axis) sorted TIL subpopulations. 4-1BB staining was performed after overnight co-incubation with cognate melanoma at 1:1 ratio. (E) TCRα 17TIL; (F) TCRβ 17TIL; (G) TCRα 135TIL; (H) TCRβ 135TIL. (I) full TCR chain sequences of neo-antigen specific candidate. Chain percentage in the different subgroup TCR repertoire is also presented. * bulk TIL including both CD4 and CD8 T-cells.

(SEQ ID NO: 10)
CATDCKNQFYF;
(SEQ ID NO: 11)
CASEEGGGFKTIF;
(SEQ ID NO: 153)
CALFGGTSYGKLTF;
(SEQ ID NO: 154)
CALSESGDAAGNKLTF;
(SEQ ID NO: 155)
CAEIPGGSYIPTF;
(SEQ ID NO: 156)
CASSLVSTPLPKETQYF;
(SEQ ID NO: 157)
CASSTPGPSAYEQYF;
(SEQ ID NO: 158)
CAEGENTEAFF;
(SEQ ID NO: 159)
CASSPWDIRTEAFF;
(SEQ ID NO: 160)
CALSESHNNAGNMLTF;
(SEQ ID NO: 161)
CAASQNTEAFF;

FIGS. 5A-E: RAS.61 mutations and HLA-A*01:01, pan-cancer and in melanoma patients (A+B) Percent of patients with HLA allele/RAS.61 mutation combination vs. expected percent assuming independence. (A) melanoma TCGA cohort; (B) pan-cancer TCGA cohort. Linear regression lines are presented. (C) HLA-A*01:01/RAS.61 is both relatively prevalent in the pan-cancer cohort and predicted to yield a neo-antigen. X axis: percent of patients with HLA-allele/RAS.61-mutant combination in TCGA pan-cancer cohort. Y axis: modified best % Rank of NetMHCpan 4.0 binding prediction; Values are calculated as: (2-% Rank) for % Rank<=2, 0 for % Rank>2. (D) Table showing counts and percent patients with HLA-A*01:01, the different RAS.61 mutations and their intersection in the TCGA cohort. (E) Frequency of HLA-A*01:01/RAS.61-mutant combination in the TCGA pan-cancer cohort. Left pie chart: HLA-A*01:01, RAS.61 mutations and combined HLA-A*01:01/RAS.61 combination frequencies in cancer patients. Right pie chart: isoform/substitution distribution among cancer patients harboring the HLA-A*01:01/RAS 0.61-mutant combination.

FIG. 6: Overlaid extracted ion chromatograms for both endogenous and heavy-peptide spike-in ILDTAGKEEY (SEQ ID NO: 1) peptides, as they were identified in targeted HLA-peptidomics of multiple tumor cell-lines bearing the HLA A*01:01/NRAS.Q61K combination. Images were produced using the Skyline software.

FIG. 7: NRAS.Q61K mutation is expressed in NRAS mutant melanoma cell-lines harboring HLA-A*01:01. The area surrounding each of the mutations site was sequenced from the cells cDNA to examine if the mutated allele of the gene is expressed

(CTGGATACAGCTGGAA/CAAGAAGAGTACAGTG -
SEQ ID NO: 248).

FIGS. 8A-C: P7 residue is free to interact with T-cell receptors according to predictions. (A) Hydrogen-bonding interactions between RAS neo-peptides ILDTAGQEEY (SEQ ID NO: 2; wild-type) and ILDTAGKEEY (SEQ ID NO: 1; mutant) and HLA-A*01:01 in cluster centroid structures. Values shown are counts of the number of hydrogen-bonding interactions formed between a given peptide residue (horizontal axis) and a HLA residue (vertical axis). HLA residues shown on the vertical axis are ordered by the total count of hydrogen interactions made with each residue. (B) Boxplot depicting the minimal distance between any sidechain atom of P7 and an HLA residue. (C) Another view at the modeled HLA-A*01:01/ILDRAGKEEY (SEQ ID NO: 1) complex. P7 sidechain faces outwards and is free to interact with the TCR.

FIG. 9: HLA-A*01:01/ILDTAGKEEY (SEQ ID NO: 1) tetramer is sensitive. IFNγ release measured in ELISA after overnight co-incubation of tetramer-sorted TIL with HLA-A*01:01 harboring B-cell IHW01161 that was pulsed with either no-peptide (DMSO only) or 10 ug/ml mutant peptide (ILDTAGKEEY-SEQ ID NO: 1). Cells were incubated in 1:1 ratio. Supernatant was diluted 1:20 before the assay was performed. Background reactivity: IFNγ concentration measured without peptide pulsing. Peptide specific reactivity: IFNγ concentration measured for the pulsed-peptide condition minus background reactivity. Error bars represent standard deviation of triplicates.

FIGS. 10A-H: CD8, CD4, tetramer and 4-1BB staining of 17TIL and 135TIL.

(A,B) CD4 and CD8 staining of bulk TIL; (A) 17TIL, (B) 135TIL. (C,D) A*01:01/ILDTAGKEEY (SEQ ID NO: 1) tetramer staining against CD4 staining of bulk TIL. The tetramer only stains CD4-cells; (C) 17TIL, (D) 135TIL. (E-H) 4-1BB staining of bulk TIL; (E,G) TIL were not exposed to target cells; (F,H) TIL were incubated with cognate melanoma overnight before staining; (E-F) 17TIL, (G-H) 135TIL.

FIGS. 11A-G: 17TIL CD4-, tetramer-positive and tetramer-negative bulk-TCRseq repertoires.

(A,B) Frequency distribution of TCRα/TCRβ chains for the CD4-TIL subpopulation, as determined via bulk-TCRseq. Chains are described at the amino-acid level. Restricting to chains that consist 1% and above of the transcripts, clear oligoclonal distributions emerge;

(A) TCRα;
(SEQ ID NO: 162)
CIVRVPGGKLIF;
(SEQ ID NO: 163)
CAASDSGAGSYQLTF;
(SEQ ID NO: 164)
CLVGATFSGNTPLVF;
(SEQ ID NO: 165)
CALRDSNSGYALNF;
(SEQ ID NO: 166)
CAFRPPTGANNLFF;
(SEQ ID NO: 167)
CILRDGASYDKVIF;
(SEQ ID NO: 168)
CASLISDGQKLLF;
(SEQ ID NO: 169)
CAVSTDSSYKLIF;
(SEQ ID NO: 170)
CATDCKNQFYF;
(SEQ ID NO: 171)
CAASVSGGTSYGKLTF;
(SEQ ID NO: 172)
CAVRPEDNFNFKYF;
(SEQ ID NO: 173)
CAVPRGGSQGNLIF;
(B) TCRβ.
(SEQ ID NO: 174)
CASSLLEHRRGDTQYF;
(SEQ ID NO: 175)
CASSLGLIGPGMNTEAFF;
(SEQ ID NO: 176)
CASSPPSGRADETDTQYF;
(SEQ ID NO: 177)
CASSPWMGNQPQHF;
(SEQ ID NO: 178)
CASSQGGSRRLDYGYTF;
(SEQ ID NO: 179)
CASSQVEGVAF;
(SEQ ID NO: 180)
CASSLVSTPLPKETQYF;
(SEQ ID NO: 181)
CASSQGPVAGENTGELFF;
(SEQ ID NO: 182)
CASSPGTEAFF;
(SEQ ID NO: 183)
CASSFLATPDTQYF;
(SEQ ID NO: 184)
CSASGQHRRYGYTF.

(C-F) Scatter plots comparing TCR chain proportions between bulk-TCRseq experiments. r denotes Pearson's correlation for the compared conditions. Tetramer-enriched chains consisting 1% and above of the CD4−/tetramer+ subpopulation are colored. ILDTAGKEEY (SEQ ID NO: 1). (C-D) CD4−vs. CD4−/tetramer−; (E-F) CD4−vs CD4−/tetramer+(C, E) TCRα, (D, F) TCRβ. (G) Percent of tetramer enriched TCRα and TCRβ chains within their cognate bulk-TCRseq tetramer+ repertoires. Black and white bar bases denote TCRα and TCRβ chains, respectively.

FIGS. 12A-G: 135TIL CD4-, tetramer-positive and tetramer-negative bulk-TCRseq repertoires.

(A,B) Frequency distribution of TCRα/TCRβ chains for the CD4-TIL subpopulation, as determined via bulk-TCRseq. Chains are described at the amino-acid level. Restricting to chains that consist 1% and above of the transcripts, clear oligoclonal distributions emerge;

(A) TCRα:
(SEQ ID NO: 185)
CAGPQDYKLSF;
(SEQ ID NO: 186)
CAVRDRNNNARLMF;
(SEQ ID NO: 187)
CAVLTGGGNKLTF;
(SEQ ID NO: 188)
CALSESHNNAGNMLTF;
(SEQ ID NO: 189)
CAMRAASNTGNQFYF;
(SEQ ID NO: 190)
CAETPNSGNTPLVF;
(SEQ ID NO: 191)
CALSEPIYNQGGKLIF;
(SEQ ID NO: 192)
CALSDLSTSGTYKYIF;
(B) TCRβ:
(SEQ ID NO: 193)
CALSDLSTSGTYKYIF;
(SEQ ID NO: 194)
CASSPAPAGAFGEQYF;
(SEQ ID NO: 195)
CASSQQGQGEAGNTIYF;
(SEQ ID NO: 196)
CAASQNTEAFF;
(SEQ ID NO: 197)
CSARDTLRGYYNEQFF;
(SEQ ID NO: 198)
CASSLGVSNQPQHF;

(C-F) Scatter plots comparing TCR chain proportions between bulk-TCRseq experiments. r denotes Pearson's correlation for the compared conditions. Tetramer-enriched chains consisting 1% and above of the CD4−/tetramer+ subpopulation are colored. ILDTAGKEEY (SEQ ID NO: 1). (C-D) CD4−vs. CD4−/tetramer−; (E-F) CD4−vs CD4−/tetramer+(C, E) TCRα, (D, F) TCRβ. (G) Percent of tetramer enriched TCRα and TCRβ chains within their cognate bulk-TCRseq tetramer+ repertoires. Black and white bar bases denote TCRα and TCRβ chains, respectively.

FIGS. 13A-J: Single-cell RNA and TCR sequencing of CD8+ 17TIL after incubation with cognate melanoma. (A) 2D visualization of transcriptome-based clusters by tSNE. Each dot corresponds to one single cell, colored according to cluster designation. TCR genes were excluded prior to clustering; (B-C) Clone mapping onto the cluster space. Presented are two neo-antigen specific clones, the three most frequent (non-neo-antigen specific) CD8+ clones, and the bulk of non-expanded clones. (B) tSNE representation of the cluster space with cells colored according to clone; (C) Bar plot showing clonal distribution across the different clusters. Numbers on top of bars designate cluster size. (D-E) Gene-expression heatmaps. Differentially expressed genes are listed to the right, with colored dots marking clusters/clones in which the gene is differentially expressed. (D) Cluster based differential-gene analysis, genes of interest are presented to the right; (E) Clone based differential-gene analysis. NRAS neoantigen-specific Clones N17.1 and N17.2 are compared against clone E17.3. All of the differential genes are presented to the right. (F-H) Violin plots comparing gene-expression signatures across the clusters. Each dot corresponds to one single cell, colored according to cluster designation. (H) Cytotoxicity signature; (G) Exhaustion signature; (H) G2/M cell cycle signature. (I) Clonal groups on the scales of cytotoxicity (Y axis) and exhaustion (X axis). (J) Expression levels of selected differentially expressed genes mapped onto tSNE representation of the cluster space. Dots represent single cells; purple hues represent low to high expression level.

FIGS. 14A-C: Single-cell TCR sequencing agrees with bulk TCRseq repertoires. (A) Few expanded clones dominate the single-cell TCR repertoire. Frequency distribution showing single-cells clones (i.e. paired αβ TCR chains) consisting of at least 1% of the single-cell repertoire. (B-C) Scatter plots comparing TCR chain proportions between single-cell TCRseq and bulk-TCRseq experiments. Single-cell TCR chain frequencies were accumulated over both singleton-chain and paired αβ cells. r denotes Pearson's correlation for the compared conditions.

FIGS. 15A-J: Singleton-chain cells stem from cognate paired ab TCR clones.

(A-E) clonal cells mapped onto the tsne plot. Cells where the full TCR sequence was detected (i.e. both a and b chains) are colored in cyan. Singleton-a cells are in yellow, and singleton-b cells are in green; (A) E17.1, (B) E17.2, (C) E17.3, (D) N17.1, (E) N17.2. (F-J) Heatmaps showing the expression levels of V and J genes, as detected in single-cell RNAseq, for singleton-chain and paired ab cells pertaining to the clones of interest; (F) E17.1, (G) E17.2, (H) E17.3, (I) N17.1, (J) N17.2.

FIG. 16: Frequency of NRAS.Q61K mutation across cancer types.

FIG. 17: Frequency of RAS.Q61K mutation across cancer types (including NRAS, KRAS and HRAS).

FIGS. 18A-C: Functional validation of individual neoantigen-specific T-cell receptors. TCRα/TCRβ chain combinations with high likelihood of forming ILDTAGKEEY/(SEQ ID NO: 1) A*01:01 TCR specificities were inferred from TCR sequencing data. Peripheral mononuclear cells (PBMC) from healthy donors were electroporated with in-vitro transcribed mRNA, coding for TCRα/TCRβ combinations of interest. N135.1={NA135.1, NB135.1}, N17.2={NA17.2, NB17.2}, N17.3={NA17.4, NB17.3}, EØ=PBMC that were electroporated without adding mRNA (negative control), CMV—reactive TCR against a CMV derived peptide that binds A*01:01 (positive control). (A) Flow cytometry plots for electroporated PBMC. Top row—mouse TCRβ constant region staining. Bottom row—ILDTAGKEEY/A*01:01 tetramer staining. Cells were stained at 6-30 hours post electroporation. (B) IFNγ release from electroporated donor PBMC as measured in ELISA. A*01:01+ B-LCL IHW01161 was pulsed with either wild-type or mutant versions of the peptide at 10 μM prior to overnight co-incubation with electroporated PBMC. Supernatants were diluted 1:20 prior to assay performance to avoid saturation. (C) In-vitro killing assay of electroporated PBMC incubated at tittered E:T ratios with GFP-tagged 17T melanoma.

### DESCRIPTION OF SPECIFIC EMBODIMENTS OF THE INVENTION

The present invention, in some embodiments thereof, relates to T cell receptors which bind to recurrent mutated neopeptides and method of identifying the recurrent mutated neopeptides.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Immunotherapeutics have curative potential in metastatic cancer, as demonstrated specifically in melanoma. The anti-tumor effect is oftentimes mediated through T-cell recognition of neo-antigens; i.e., HLA-presented mutation-bearing peptides. With few exceptions, identified neo-antigens from responders stem from private mutations, and thus cannot be generalized beyond the individual patient. By definition, “recurrent neo-antigens” are such that are shared among groups of patients. Naturally, these are antigens that derive from common driver mutations and present on common HLA alleles. Moreover, due to the clonality of driver mutations, they are expected to present uniformly within tumors and across metastases. Recurrent neo-antigens should therefore have great clinical value, as they may serve for the development of effective, tumor-specific, “off-the-shelf” therapies.

The present inventors have now combined a novel bioinformatic analysis on the TCGA melanoma cohort which considers both the presence of recurrent mutations and the patient HLA allotype in combination with binding predictions, thus directing the discovery of promising recurrently presented neo-antigen candidates. This revealed that 2.2% of the patients possess the HLA-A*01:01/NRAS.Q61K combination. Using HLA-peptidomics, the present inventors were able to directly demonstrate the presentation of HLA-A*01:01/NRAS.Q61K-derived hotspot neo-antigen on multiple tumor cell-lines. Tumor infiltrating lymphocytes (TILs) from two unrelated individuals with tumors bearing the A*01:01/NRAS.Q61K combination showed specific reactivity toward the mutated peptide. Tetramer-sorted T-cells from these pools were able to eliminate their cognate melanoma in a dose-dependent manner. T-cell receptor (TCR) sequencing of tetramer- and 4-1BB-positive TILs suggests that the neo-antigen is immuno-dominant in the bulk TIL population.

The present inventors thus predict that HLA-A*01:01/NRAS.Q61K hotspot neo-antigen is a robust immunogenic target that is relevant for thousands of patients yearly.

Thus, according to a first aspect of the present invention, there is provided a method of selecting an HLA-presented neoantigen which can be targeted in a cancer-immunotherapy treatment, the method comprising:

(a) analyzing the frequency of occurrence of a cancer-associated mutated protein in the context of an individual HLA allele in a plurality of cancer patients; and

(b) determining the binding affinity of peptides of 8-14 amino acids in length derived from the cancer-associated mutated protein to the individual HLA allele, wherein the peptides comprise a mutation compared to the wild-type protein,

wherein a candidate peptide which binds with an affinity above a first predetermined level to an HLA allele having a frequency of occurrence above a second predetermined level, is selected as an HLA-presented neoantigen that can be targeted in a cancer-immunotherapy treatment.

As used herein the term “neoantigen” is an epitope that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

In one embodiment, the neoantigen is a short peptide that is bound to a class I or II MHC receptor thus forming a ternary complex that can be recognized by a T-cell bearing a matching T-cell receptor binding to the MHC/peptide complex with appropriate affinity. Peptides binding to MHC class I molecules are typically about 8-14 amino acids in length. T-cell epitopes that bind to MHC class II molecules are typically about 12-30 amino acids in length. In the case of peptides that bind to MHC class II molecules, the same peptide and corresponding T cell epitope may share a common core segment, but differ in the overall length due to flanking sequences of differing lengths upstream of the amino-terminus of the core sequence and downstream of its carboxy terminus, respectively. A T-cell epitope may be classified as an antigen if it elicits an immune response.

Proteins from which the neoantigens are derived comprise cancer-associated modifications. Exemplary modifications include, but are not limited to cancer associated mutations and cancer-associated phosphorylation patterns.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. Preferably a mutation is a non-synonymous mutation. The term “non-synonymous mutation” refers to a mutation, preferably a nucleotide substitution, which does result in an amino acid change such as an amino acid substitution in the translation product.

According to the invention, the term “mutation” includes point mutations, Indels, fusions, chromothripsis and RNA edits.

According to a specific embodiment, the mutation is a point mutation—i.e. a single amino acid substitution.

According to the invention, the term “Indel” describes a special mutation class, defined as a mutation resulting in a colocalized insertion and deletion and a net gain or loss in nucleotides. In coding regions of the genome, unless the length of an indel is a multiple of 3, they produce a frameshift mutation. Indels can be contrasted with a point mutation; where an Indel inserts and deletes nucleotides from a sequence, a point mutation is a form of substitution that replaces one of the nucleotides. In one embodiment, the indel is a frameshift deletion mutation. In another embodiment, the indel is a frameshift insertion mutation.

Fusions can generate hybrid genes formed from two previously separate genes. It can occur as the result of a translocation, interstitial deletion, or chromosomal inversion. Often, fusion genes are oncogenes. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. Oncogenic fusion transcripts may also be caused by trans-splicing or read-through events.

According to the invention, the term “chromothripsis” refers to a genetic phenomenon by which specific regions of the genome are shattered and then stitched together via a single devastating event.

According to the invention, the term “RNA edit” or “RNA editing” refers to molecular processes in which the information content in an RNA molecule is altered through a chemical change in the base makeup. RNA editing includes nucleoside modifications such as cytidine (C) to uridine (U) and adenosine (A) to inosine (I) deaminations, as well as non-templated nucleotide additions and insertions. RNA editing in mRNAs effectively alters the amino acid sequence of the encoded protein so that it differs from that predicted by the genomic DNA sequence.

Preferably, the mutations are non-synonymous mutations, preferably non-synonymous mutations of proteins expressed in a tumor or cancer cell.

In a particular embodiment, the protein which expresses a cancer-related modification pattern is expressed in melanoma cells, lung cancer cells, renal cancer cells or Head and neck squamous carcinoma cells.

Preferably, the protein which expresses a cancer-related modification pattern is expressed in melanoma cells.

Preferably, the protein which expresses a cancer-related modification pattern is a human protein.

Examples of proteins which may express cancer related modification patterns include those that are members of the RAS family—e.g. Neuroblastoma RAS Viral (V-Ras) Oncogene Homolog (NRAS; UniProtKB—P01111), Kirsten rat sarcoma viral oncogene homolog (KRAS; UniProtKB—P01116) and Harvey Rat Sarcoma Viral Oncogene Homolog (HRAS, UniProtKB—P01112).

Specific contemplated NRAS variants include Q61K, Q61R, Q61L and Q61H.

Another example of a cancer associated mutated protein is a RAF kinase—e.g. B-RAF UniProtKB—P15056.

Specific B-RAF variants include V600E, V600M, G466E, H725Y, K601E and V600G.

Other examples include, but are not limited to kallikrein 4, papillomavirus binding factor (PBF), preferentially expressed antigen of melanoma (PRAME), Wilms' tumor-1 (WT1), Hydroxysteroid Dehydrogenase Like 1 (HSDL1), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule EpCAM), ovarian and uterine carcinoma antigen (CA125), folate receptor a, sperm protein 17, tumor-associated differentially expressed gene-12 (TADG-12), mucin-16 (MUC-16), L1 cell adhesion molecule (L1CAM), mannan-MUC-1, Human endogenous retrovirus K (HERV-K-MEL), Kita-kyushu lung cancer antigen-1 (KK-LC-1), human cancer/testis antigen (KM-HN-1), cancer testis antigen (LAGE-1), melanoma antigen-A1 (MAGE-A1), Sperm surface zona pellucida binding protein (Sp17), Synovial Sarcoma, X Breakpoint 4 (SSX-4), Transient axonal glycoprotein-1 (TAG-1), Transient axonal glycoprotein-2 (TAG-2), Enabled Homolog (ENAH), mammoglobin-A, NY-BR-1, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin 1 (MUC1), p62, survivin, lymphocyte common antigen (CD45), Dickkopf WNT Signaling Pathway Inhibitor 1 (DKK1), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor 1 Homolog (COA-1), O-linked (3-N-acetylglucosamine transferase (OGT), Osteosarcoma Amplified 9, Endoplasmic Reticulum Lectin (OS-9), Transforming Growth Factor Beta Receptor 2 (TGF-betaRll), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (ME1), Nuclear Transcription Factor Y Subunit C (NFYC), G Antigen 1,3 (GAGE-1,3), melanoma antigen-A6 (MAGE-A6), cancer testis antigen XAGE-1b, six transmembrane epithelial antigen of the prostate 1 (STEAP1), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTC1), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FN1), Growth Arrest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T-Cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDXS), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OA1), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gp100), N-acetylglucosaminyltransferase V gene (GnTVf), Lymphocyte Antigen 6 Complex Locus K (LY6K), melanoma antigen-A10 (MAGE-A10), melanoma antigen-A12 (MAGE-A12), melanoma antigen-C2 (MAGE-C2), melanoma antigen NA88-A, Taxol-resistant-associated protein 3 (TRAG-3), BDZ1 binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B,) promyelocytic leukemia/retinoic acid receptor alpha fusion protein (pml-RARalpha), melanoma antigen-C1 (MAGE-C1), membrane protein alternative spliced isoform (D393-CD20), melanoma antigen-A4 (MAGE-A4), or melanoma antigen-A3 (MAGE-A3).

Additional examples of proteins that may express cancer related modification patterns are known in the art and are described, for example, in Reuschenbach et al., Cancer Immunol. Immunother. 58:1535-1544 (2009); Parmiani et al., J. Nat. Cancer Inst. 94:805-818 (2002); Zarour et al., Cancer Medicine. (2003); Bright et al., Hum. Vaccin. Immunother. 10:3297-3305 (2014); Wurz et al., Ther. Adv. Med. Oncol. 8:4-31 (2016); Criscitiello, Breast Care 7:262-266 (2012); Chester et al., J. Immunother. Cancer 3:7 (2015); Li et al., Mol. Med. Report 1:589-594 (2008); Liu et al., J. Hematol. Oncol. 3:7 (2010); Bertino et al., Biomed. Res. Int. 731469 (2015); and Suri et al., World J. Gastrointest. Oncol. 7:492-502 (2015).

In one embodiment, the mutations are cancer specific somatic mutations.

Methods for detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In one embodiment, the step of identifying cancer specific somatic mutations or identifying sequence differences involves using next generation sequencing (NGS).

In one embodiment, the step of identifying cancer specific somatic mutations or identifying sequence differences comprises sequencing genomic DNA and/or RNA of the tumor specimen.

To reveal cancer specific somatic mutations or sequence differences the sequence information obtained from the tumor specimen is preferably compared with a reference such as sequence information obtained from sequencing nucleic acid such as DNA or RNA of normal non-cancerous cells such as germline cells which may either be obtained from the patient or a different individual. In one embodiment, normal genomic germline DNA is obtained from peripheral blood mononuclear cells (PBMCs).

The term “genome” relates to the total amount of genetic information in the chromosomes of an organism or a cell.

The term “exome” refers to part of the genome of an organism formed by exons, which are coding portions of expressed genes. The exome provides the genetic blueprint used in the synthesis of proteins and other functional gene products. It is the most functionally relevant part of the genome and, therefore, it is most likely to contribute to the phenotype of an organism. The exome of the human genome is estimated to comprise 1.5% of the total genome (Ng, P C et al., PLoS Gen., 4(8): 1-15, 2008).

The term “transcriptome” relates to the set of all RNA molecules, including mRNA, rRNA, tRNA, and other non-coding RNA produced in one cell or a population of cells. In context of the present invention the transcriptome means the set of all RNA molecules produced in one cell, a population of cells, preferably a population of cancer cells, or all cells of a given individual at a certain time point.

According to the invention, a “reference” may be used to correlate and compare the results obtained in the methods of the invention from a tumor specimen. Typically the “reference” may be obtained on the basis of one or more normal specimens, in particular specimens which are not affected by a cancer disease, either obtained from a patient or one or more different individuals, preferably healthy individuals, in particular individuals of the same species. A “reference” can be determined empirically by testing a sufficiently large number of normal specimens.

Any suitable sequencing method can be used according to the invention for determining mutations, Next Generation Sequencing (NGS) technologies being preferred. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention e.g. those described in detail in Zhang et al. 2011: The impact of next-generation sequencing on genomics. J. Genet Genomics 38 (3), 95-109; or in Voelkerding et al. 2009: Next generation sequencing: From basic research to diagnostics. Clinical chemistry 55, 641-658. Non-limiting examples of such NGS technologies/platforms are:

1) The sequencing-by-synthesis technology known as pyrosequencing implemented e.g. in the GS-FLX 454 Genome Sequencer™ of Roche-associated company 454 Life Sciences (Branford, Conn.), first described in Ronaghi et al. 1998: A sequencing method based on real-time pyrophosphate”. Science 281 (5375), 363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand.

2) The sequencing-by-synthesis approaches developed by Solexa (now part of Illumina Inc., San Diego, Calif.) which is based on reversible dye-terminators and implemented e.g. in the Illumina/Solexa Genome Analyzer™ and in the Illumina HiSeq 2000 Genome Analyze™. In this technology, all four nucleotides are added simultaneously into oligo-primed cluster fragments in flow-cell channels along with DNA polymerase. Bridge amplification extends cluster strands with all four fluorescently labeled nucleotides for sequencing.

3) Sequencing-by-ligation approaches, e.g. implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, he Polonator™ G.007 platform of Dover Systems (Salem, N.H.) also employs a sequencing-by-ligation approach by using a randomly arrayed, bead-based, emulsion PCR to amplify DNA fragments for parallel sequencing.

4) Single-molecule sequencing technologies such as e.g. implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™) and Genovoxx (AnyGene™)

5) Nano-technologies for single-molecule sequencing in which various nanostructures are used which are e.g. arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™TM^{T}) platforms developed by Nabsys (Providence, R.I.), and the proprietary ligase-based DNA sequencing platform with DNA nanoball (DNB) technology called combinatorial probe-anchor ligation (cPALTM)

6) Electron microscopy based technologies for single-molecule sequencing, e.g. those developed by LightSpeed Genomics (Sunnyvale, Calif.) and Halcyon Molecular (Redwood City, Calif.) [0170] 7) Ion semiconductor sequencing which is based on the detection of hydrogen ions that are released during the polymerisation of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

Preferably, DNA and RNA preparations serve as starting material for NGS. Such nucleic acids can be easily obtained from samples such as biological material, e.g. from fresh, flash-frozen or formalin-fixed paraffin embedded tumor tissues (FFPE) or from freshly isolated cells or from CTCs which are present in the peripheral blood of patients. Normal non-mutated genomic DNA or RNA can be extracted from normal, somatic tissue, however germline cells are preferred in the context of the present invention. Germline DNA or RNA may be extracted from peripheral blood mononuclear cells (PBMCs) in patients with non-hematological malignancies. Although nucleic acids extracted from FFPE tissues or freshly isolated single cells are highly fragmented, they are suitable for NGS applications.

Several targeted NGS methods for exome sequencing are described in the literature (for review see e.g. Teer and Mullikin 2010: Human Mol Genet 19 (2), R145-51), all of which can be used in conjunction with the present invention. Many of these methods (described e.g. as genome capture, genome partitioning, genome enrichment etc.) use hybridization techniques and include array-based (e.g. Hodges et al. 2007: Nat. Genet. 39, 1522-1527) and liquid-based (e.g. Choi et al. 2009: Proc. Natl. Acad. Sci USA 106, 19096-19101) hybridization approaches. Commercial kits for DNA sample preparation and subsequent exome capture are also available: for example, Illumina Inc. (San Diego, Calif.) offers the TruSee™. DNA Sample Preparation Kit and the Exome Enrichment Kit TruSeq™ Exome Enrichment Kit.

Methods for identifying disease-specific phosphorylation patterns are known in the art and include for example stable isotope labeling with amino acids in cell culture (SILAC), RRPA, and phospho-specific Western blots.

In a preferred embodiment, the HLA allele is a class I HLA allele. In particular embodiments, the class I HLA allele is an HLA-A allele or an HLA-B allele. In a preferred embodiment, the HLA allele is a class II HLA allele. Sequences of class I and class II HLA alleles can be found in the IPD-EVIGT/HLA Database. Exemplary HLA alleles include but are not limited to A*01:01, A*02:01, A*02:03, A*02:04, A*02:07, A*03:01, A*24:02, A*29:02, A*31:01, A*68:02, B*35:01, B*44:02, B*44:03, B*51:01, B*54:01 or B57:01 In particular embodiments, the HLA allele is HLA-A*01:01.

Subject specific HLA alleles or HLA genotype of a subject may be determined by any method known in the art. In a particular embodiment, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

Preferably a cancer-associated mutated protein in the context of an individual HLA allele is selected which has a high frequency in a predetermined number of cancer patients (e.g. at least greater than 200, 300, 400, 500, 600, 700, 800, 900, 1000, 2000 or more).

The cancer patient group may be suffering from the same cancer type—melanoma or may be part of a pan-cancer group suffering from a number of different cancer types.

The cancer patient group may include melanoma patients, thyroid cancer patients, pheochromocytoma patients, seminoma patients, stomach adenocarcinoma patients, cholangiocarcinoma patients, pancreatic adenocarcinoma patients, colorectal adenocarcinoma, leukemia patients, bladder urothelial carcinoma patients, endometrial carcinoma patients, thymic epithelial tumor patients, non-small cell lung cancer patients, sarcoma patients, ovarian cancer patients and prostate cancer patients, or any combination of the above described cancer patients.

In one embodiment, the cancer patient group includes only melanoma cancer patients.

It will be appreciated that the HLA status may have a high frequency in the group and/or there is a high frequency of the presence of the particular mutation in that group. Preferably, the HLA status frequency is high (e.g. over 0.5%, 1%, 2%, 3%, 4%, 5%, 6%, 7%, 8%, 9%, 10%) and the frequency of the particular mutation in that group is also high (e.g. over 0.5%, 1%, 2%, 3%, 4%, 5%, 6%, 7%, 8%, 9%, 10% a).

Once the HLA allele and the particular mutated protein have been selected, the binding affinity of peptides (which comprise the selected mutation) of 8-14 amino acids in length derived from the selected cancer-associated mutated protein to the selected HLA allele are analyzed.

Methods of analyzing binding affinity of peptides to HLA alleles are known in the art.

In one embodiment, the binding affinity can be predicted using a prediction algorithm for HLA binding. Such prediction algorithms include but are not limited to NetMHC, NetMHC II, NetMHCpan, IEDB Analysis Resource (URL immuneepitope.org), RankPep, PREDEP, SVMHC, Epipredict, HLABinding, and others (see e.g., J Immunol Methods 2011; 374:1-4).

Using such predictions, a list of candidate neoantigens can be generated that bind with an affinity above a predetermined amount to the HLA. According to a particular embodiment, only candidate peptides that bind with a % Rank≤0.5 (default parameters of NetMHCpan) are selected, or a corresponding level using a different prediction algorithm. According to another embodiment, candidate peptides are selected whose binding can be characterized as % Rank≤2 (default parameters of NetMHCpan), or a corresponding level using a different prediction algorithm.

It will be appreciated that the binding affinity may be lower than the above mentioned levels if the HLA allele frequency and/or the frequency of the mutation is high.

Thus, it will be understood that it is the combination of the three parameters—frequency of HLA allele, frequency of occurrence of the mutation and the binding affinity which together dictate the selection of candidate neoantigens, and is not based on only a single parameter. Thus, the predetermined amount for any one of the parameters is not a fixed amount but rather is fluid and can be changed according to the levels of the other two parameters.

Neoantigen candidate peptides from such a list can then be recommended as targets in cancer-immunotherapy treatments, which are further described herein below.

Optionally, the candidate peptides are corroborated by ascertaining that the candidate peptide binds to the specified HLA allele in at least one cancer patient.

This may be carried out using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

In a particular embodiment, the analysis may be determined using liquid chromatography and tandem mass spectrometry (LC−MS/MS) and/or HPLC—see for example Kalaora et al., Oncotarget. 2016 Feb. 2; 7(5): 5110-5117, the contents of which being incorporated herein by reference.

The reactivity of the selected neoantigens can then be assessed as further described herein below.

Firstly, the neoantigens are synthesized and loaded onto antigen presenting cells (APCs) under conditions that allow the presentation of the epitopes on the surface of the APCs.

Antigen presenting cells (APC) are cells which present peptide fragments of protein antigens in association with HLA (MHC) molecules on their cell surface. Some APCs may activate antigen specific T cells.

Preferably, the APC can also stimulate CD4+ helper T cells as well as cytotoxic T cells.

Examples of APCs include, but are not limited to dendritic cells, macrophages, Langerhans cells and B

According to a particular embodiment, the APCs are dendritic cells or B cells. Most preferable are B cells.

In one embodiment, the APCs are immortalized—i.e. a transformed cell line, such as Epstein Barr Virus (EBV)-transformed B cells.

In one embodiment, the APCs may be genetically modified to express HLA alleles restricted to the subject who is being tested.

B cells that are HLA deficient (e.g. B721.221) can be used so that the system is “clean” from non-relevant HLAs. Particular HLAs (e.g. those relevant to a particular subject) can be overexpressed in such B cells.

An exemplary method for deleting/inactivating endogenous class I or class II genes in antigen presenting cells which express non-relevant HLA alleles is CRISPR-Cas9 mediated genome editing.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50. According to an embodiment of this aspect of the present invention, the peptides are purified (e.g. >80% purity, >85% purity, >90% purity, >95% purity).

According to a particular embodiment, the peptides are attached to cell penetrating moieties.

As used herein, the term “cell penetrating moiety” refers to a moiety (e.g. a peptide, a lipid, such as palmitic acid) which enhances translocation of an attached peptide across a cell membrane.

According to one embodiment, the penetrating moiety is a peptide and is attached to the peptides spanning the disease-associated modification (either directly or non-directly) via a peptide bond. In one embodiment, the penetrating agent is attached to the N terminus of the peptide. In another embodiment, the penetrating agent is attached to the C terminus of the peptide. In still another embodiment, the penetrating agent is attached in the middle of the peptide (i.e. not at the terminii).

Typically, peptide-penetrating agents have an amino acid composition containing either a high relative abundance of positively charged amino acids such as lysine or arginine, or have sequences that contain an alternating pattern of polar/charged amino acids and non-polar, hydrophobic amino acids.

Another method of enhancing cell penetration is via N-terminal myristoilation. In this protein modification, a myristoyl group (derived from myristic acid) is covalently attached via an amide bond to the alpha-amino group of an N-terminal amino acid of the peptide.

As mentioned, the peptides of this aspect of the present invention are loaded onto the APCs under conditions that allow them to be presented on the surface of the APCs.

To be presented on the surface of the APCs, they have to cross the APC cell membrane and loaded onto newly synthesized HLA class I or II receptors. Formed HLA-peptide complexes are translocated onto the cell membrane, where they are readily available for T-cell recognition.

In one embodiment, the peptides are incubated with the APCs in a medium which maintains the APCs in a viable state (e.g. RPMI) for an amount of time between 12-48 hours, 12-24 hours, 6-48 hours or 8-48 hours. The concentration of the peptide is preferably between 10-50 μM and more preferably between 10-30 μM during the loading stage.

Next, activation of CD4+ or CD8+ T cells may be determined. Methods for detecting specific T cell activation include detecting the proliferation of T cells, the production of cytokines (e.g., lymphokines, interferon gamma, TNF alpha), or the generation of cytolytic activity. For CD4+ T cells, a preferred method for detecting specific T cell activation is the detection of the proliferation of T cells. For CD8+ T cells, a preferred method for detecting specific T cell activation is the detection of the generation of cytolytic activity.

According to a particular embodiment, in order to determine the reactivity of the peptides, an ELISPOT assay may be carried out, where the CD8+ CTL response, which can be assessed by measuring IFN-gamma production by antigen-specific effector cells, is quantitated by measuring the number of Spot Forming Units (SFU) under a stereomicroscope (Rininsland et al., (2000) J Immunol Methods: 240(1-2):143-155). In this assay, antigen-presenting cells (APC) are immobilized on the plastic surface of a micro titer well, and effector T cells are added at various effector:target ratios. Antigen presenting cells are preferably B cells or dendritic cells. The binding of APC's by antigen-specific effector cells triggers the production of cytokines including IFN-gamma by the effector cells (Murali-Krishna et al., (1998) Adv Exp Med Biol.: 452:123-142). In one embodiment subject specific T cells are used in the ELISPOT assay. The amount of soluble IFNγ secreted from the TILs may also be measured by ELISA assay (e.g. Biolegend).

Another method for determining the reactivity of the peptides is by direct determination of cell lysis as measured by the classical assay for CTL activity namely the chromium release assay (Walker et al., (1987) Nature: 328:345-348; Scheibenbogen et al., (2000) J Immunol Methods: 244(1-2):81-89). Effector Cytotoxic T Lymphocytes (CTL) bind targets bearing antigenic peptide on Class I MHC and signal the targets to undergo apoptosis. If the targets are labeled with ⁵¹Chromium before the CTL are added, the amount of ⁵¹Cr released into the supernatant is proportional to the number of targets killed. Antigen-specific lysis is calculated by comparing lysis of target cells expressing disease or control antigens in the presence or absence of patient effector cells, and is usually expressed as the %-specific lysis. Percent specific cytotoxicity is calculated by (specific release-spontaneous release)/(maximum release-spontaneous release) and may be 20%-85% for a positive assay. Percent specific cytotoxicity is usually determined at several ratios of effector (CTL) to target cells (E:T). Additionally, the standard lytic assay is qualitative and must rely on a limiting dilution analysis (LDA) for quantitative results, and the LDA frequently underestimates the true level of CTL response. Although CTL can each kill many targets in vivo, in vitro this assay requires numbers of CTL equal to or greater than the number of targets for detectable killing. In one embodiment CTL responses are measured by the chromium release assay, monitoring the ability of T cells (Effector cells) to lyse radiolabelled HLA matched “target cells” that express the appropriate antigen-MHC complex.

It will be appreciated that by uncovering promising recurrently presented neo-antigen candidates, the present inventors are now able to select which patients are promising candidates for such therapy. The patients may be selected in the absence of concrete knowledge (experimental confirmation) as to whether they present the peptide or not.

Thus, according to another aspect of the disclosure, there is provided a method of selecting a subject suffering from cancer for cancer-immunotherapy treatment comprising:

(a) ascertaining the HLA profile of a subject;

(b) determining whether the subject comprises a genome which encodes a cancer-associated mutated protein; wherein the subject is selected for treatment when:

(i) the HLA profile of the subject occurs with a frequency above a predetermined level in a plurality of cancer patients;

(ii) the cancer-associated mutated protein of the subject occurs with a frequency above a predetermined level in a plurality of cancer patients; and

(iii) at least one peptide of 8-14 amino acids in length derived from the cancer-associated mutated protein binds to an HLA which is of the identical allele to the subject above a predetermined level, wherein the peptide comprises a mutation compared to the wild-type protein.

Methods of determining the HLA profile of a subject have been described herein above.

Methods of determining whether the subject comprises a genome which encodes a cancer-associated mutated protein are known and include both polypeptide-based methods and polynucleotide based methods, as further described herein above.

Subjects who are of a frequently-occurring HLA and who express a frequently occurring mutation in a cancer associated mutated protein are likely candidates for selection. If a peptide of 8-14 amino acids in length derived from the cancer-associated mutated protein binds with high affinity (as further described herein above) to the individual HLA allele, then a cancer immunity therapy treatment which targets the peptide may be recommended for that subject.

Candidate subjects are those suffering from metastatic cancer. Examples of cancers include but are not limited to melanoma, colon cancer, breast cancer, thyroid cancer, stomach cancer, colorectal cancer, leukemia cancer, bladder cancer, lung cancer, ovarian cancer, breast cancer and prostate cancer.

Agents that can be used in cancer immunotherapy treatment include, but are not limited to vaccines, antibodies and populations of T cells expressing a receptor that targets the T cell epitope.

As used herein, the term “vaccine” refers to a pharmaceutical preparation (pharmaceutical composition) or product that upon administration induces an immune response, in particular a cellular immune response, which recognizes and attacks a pathogen or a diseased cell such as a cancer cell.

The vaccine may be used for the prevention or treatment of a disease such as cancer (e.g. melanoma). The term “personalized cancer vaccine” or “individualized cancer vaccine” concerns a particular cancer patient and means that a cancer vaccine is adapted to the needs or special circumstances of an individual cancer patient.

In one embodiment, the vaccine comprises a peptide predicted as being an advantageous target by the methods of the invention or a nucleic acid, preferably RNA, encoding the peptide or polypeptide.

The cancer vaccines provided according to the invention when administered to a patient provide one or more T cell epitopes suitable for stimulating, priming and/or expanding T cells specific for the patient's tumor. The T cells are preferably directed against cells expressing antigens from which the T cell epitopes are derived. Thus, the vaccines described herein are preferably capable of inducing or promoting a cellular response, preferably cytotoxic T cell activity, against a cancer disease characterized by presentation of one or more tumor-associated neoantigens. Since a vaccine provided according to the present invention will target cancer specific mutations it will be specific for the patient's tumor.

The vaccine can comprise one or more T cell epitopes identified according to the methods described herein, such as 2 or more, 5 or more, 10 or more, 15 or more, 20 or more, 25 or more, 30 or more and preferably up to 60, up to 55, up to 50, up to 45, up to 40, up to 35 or up to 30 T cell epitopes.

According to a particular embodiment, the T cell epitope consists of a sequence as set forth in any one of SEQ ID NOs: 1 and 12-132.

According to a more particular embodiment, the T cell epitope consists of a sequence as set forth in any one of SEQ ID NOs: 1 and 12-28.

Presentation of these epitopes by cells of a patient, in particular antigen presenting cells, preferably results in T cells targeting the epitopes when bound to MHC and thus, the patient's tumor, preferably the primary tumor as well as tumor metastases, expressing antigens from which the T cell epitopes are derived and presenting the same epitopes on the surface of the tumor cells.

The peptides determined according to the methods of the present invention may be ranked for their usability as epitopes for cancer vaccination. Thus, in one aspect, the method of the invention comprises a manual or computer-based analytical process in which the identified peptides are analyzed and selected for their usability in the respective vaccine to be provided. In a preferred embodiment, the analytical process is a computational algorithm-based process. Preferably, the analytical process comprises determining and/or ranking epitopes according to a prediction of their capacity of being immunogenic.

The vaccines of the present invention may further comprise an adjuvant.

The term “adjuvant” as used herein refers to an agent that nonspecifically increases an immune response to a particular antigen thereby reducing the quantity of antigen necessary in any given vaccine and/or the frequency of injection necessary in order to generate an adequate immune response to the antigen of interest. Suitable adjuvants for use herein include, but are not limited to, poly IC; synthetic oligodeoxynucleotides (ODNs) with a CpG motif; modified polyinosinic:polycytidylic acid (Poly-IC) including, but not limited to, Poly-IC/LC (Hiltonol) and Poly-IC12U (Ampligen); Poly-K; carboxymethyl cellulose (CMC); Adjuvant 65 (containing peanut oil, mannide monooleate, an aluminum monostearate); Freund's complete or incomplete adjuvant; mineral gels such as aluminum hydroxide, aluminum phosphate, and alum; surfactants such as hexadecylamine, octadecylamine, lysolecithin, dimethyldioctadecylammonium bromide, N,N-dioctadecyl-N′,N″-bis(2-hydroxymethyl)propanediamine, methoxyhexadecylglyerol and pluronic polyols; polyanions such as pyran, dextran sulfate, polyacrylic acid, and carbopol; peptides such as muramyl dipeptide, dimethylglycine and tuftsin; and oil emulsions. The adjuvants of the present invention may include nucleic acids based on inosine and cytosine such as poly I:poly C; poly IC; poly dC; poly dI; poly dIC; Poly-IC/LC; Poly-K; and Poly-IC12U as well as oligodeoxynucleotides (ODNs) with a CpG motif, CMC and any other combinations of complementary double stranded IC sequences or chemically modified nucleic acids such as thiolated poly IC as described in U.S. Pat. Nos. 6,008,334; 3,679,654 and 3,725,545.

The peptide-based vaccines disclosed herein are capable of being used in combination with another therapeutic. Examples of therapeutics that can be used in conjunction with the vaccines disclosed herein include, but are not limited to: immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1; and immunomodulatory drugs including, but not limited to, lenalidomide (Revlimid).

In addition, the peptide-based vaccines disclosed herein may be administered for cancer treatment in combination with chemotherapy in regimens that do not inhibit the immune system including, but not limited to, low dose cyclophosphamide and taxol. The vaccines may also be administered for cancer in combination with therapeutic antibodies including, but not limited to, anti-HER2/neu (Herceptin) and anti-CD20 (Rituxan).

The peptide-based vaccines can be administered for treatment of chronic infections in combination with drugs used to treat the particular type of infection including, but not limited to, anti-viral drugs, anti-retroviral drugs, anti-malarial drugs, etc.

In one embodiment, the agents of this aspect of the present invention are administered together with immune checkpoint inhibitors.

As used herein, the phrase “immune checkpoint inhibitor” refers to a compound capable of inhibiting the function of an immune checkpoint protein. Inhibition includes reduction of function and full blockade. In particular the immune checkpoint protein is a human immune checkpoint protein. Thus the immune checkpoint protein inhibitor preferably is an inhibitor of a human immune checkpoint protein. Immune checkpoint proteins are described in the art (see for instance Pardoll, 2012. Nature Rev. cancer 12: 252-264). The designation immune checkpoint includes the experimental demonstration of stimulation of an antigen-receptor triggered T lymphocyte response by inhibition of the immune checkpoint protein in vitro or in vivo, e.g. mice deficient in expression of the immune checkpoint protein demonstrate enhanced antigen-specific T lymphocyte responses or signs of autoimmunity (such as disclosed in Waterhouse et al., 1995. Science 270:985-988; Nishimura et al., 1999. Immunity 11:141-151). It may also include demonstration of inhibition of antigen-receptor triggered CD4+ or CD8+ T cell responses due to deliberate stimulation of the immune checkpoint protein in vitro or in vivo (e.g. Zhu et al., 2005. Nature Immunol. 6:1245-1252).

Preferred immune checkpoint protein inhibitors are antibodies that specifically recognize immune checkpoint proteins. A number of CTLA-4, PD1, PDL-1, PD-L2, LAG-3, BTLA, B7H3, B7H4, TIM3 and KIR inhibitors are known and in analogy of these known immune checkpoint protein inhibitors, alternative immune checkpoint inhibitors may be developed in the (near) future. For example ipilimumab is a fully human CTLA-4 blocking antibody presently marketed under the name Yervoy (Bristol-Myers Squibb). A second CTLA-4 inhibitor is tremelimumab (referenced in Ribas et al, 2013, J. Clin. Oncol. 31:616-22). Examples of PD-1 inhibitors include without limitation humanized antibodies blocking human PD-1 such as lambrolizumab (e.g. disclosed as hPD109A and its humanized derivatives h409A11, h409A16 and h409A17 in WO2008/156712; Hamid et al., N. Engl. J. Med. 369: 134-144 2013), or pidilizumab (disclosed in Rosenblatt et al., 2011. J. Immunother. 34:409-18), as well as fully human antibodies such as nivolumab (previously known as MDX-1106 or BMS-936558, Topalian et al., 2012. N. Eng. J. Med. 366:2443-2454, disclosed in U.S. Pat. No. 8,008,449 B2). Other PD-1 inhibitors may include presentations of soluble PD-1 ligand including without limitation PD-L2 Fc fusion protein also known as B7-DC-Ig or AMP-244 (disclosed in Mkrtichyan M, et al. J Immunol. 189:2338-47 2012) and other PD-1 inhibitors presently under investigation and/or development for use in therapy. In addition, immune checkpoint inhibitors may include without limitation humanized or fully human antibodies blocking PD-L such as MEDI-4736 (disclosed in WO2011066389 A1), MPDL3280A (disclosed in U.S. Pat. No. 8,217,149 B2) and MIH1 (Affymetrix obtainable via eBioscience (16.5983.82)) and other PD-L1 inhibitors presently under investigation. According to this invention an immune checkpoint inhibitor is preferably selected from a CTLA-4, PD-1 or PD-L1 inhibitor, such as selected from the known CTLA-4, PD-1 or PD-L1 inhibitors mentioned above (ipilimumab, tremelimumab, labrolizumab, nivolumab, pidilizumab, AMP-244, MEDI-4736, MPDL3280A, MIH1). Known inhibitors of these immune checkpoint proteins may be used as such or analogues may be used, in particular chimerized, humanized or human forms of antibodies.

Other agents used in the arsenal of cancer immunotherapy agents include T cell populations that are capable of binding to the peptide epitopes described herein for adoptive cell therapy (ACT).

ACT refers to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73),In one embodiment TCRs are selected for administering to a subject based on binding to neoantigens as identified herein. In one embodiment T cells are expanded using methods known in the art. Expanded T cells that express tumor specific TCRs may be administered back to a subject. In another embodiment PBMCs are transduced or transfected with polynucleotides for expression of TCRs and administered to a subject. T cells expressing TCRs specific to neoantigens are expanded and administered back to a subject. In one embodiment T cells that express TCRs for the neoantigens uncovered using the methods described herein, that result in cytolytic activity when incubated with autologous tumor tissue are expanded and administered to a subject.

Thus, for example the present invention contemplates use of T cell populations comprising T cell receptors that can bind to at least one of the peptide epitopes having the sequences as set forth in SEQ ID NOs: 1 and 12-132 and have antigenic specificity towards the corresponding mutated polypeptides.

Alternatively, or additionally, the present invention contemplates use of T cell populations expressing chimeric antibodies (CAR-T cells) on the surface thereof that can bind to at least one of the peptide epitopes having the sequences as set forth in SEQ ID NOs: 1 and 12-132 and have antigenic specificity towards the corresponding mutated polypeptides.

The phrase “antigenic specificity,” as used herein, means that the TCR (or antibody) can specifically bind to and immunologically recognize mutated target, e.g., mutated NRAS or BRAF, with high avidity. For example, a TCR may be considered to have “antigenic specificity” for mutated target if T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-gamma upon co-culture with (a) antigen-negative HLA-A*01:01⁺ target cells pulsed with a low concentration of mutated target peptide (e.g., about 0.05 ng/mL to about 5 ng/mL, 0.05 ng/mL, 0.1 ng/mL, 0.5 ng/mL, 1 ng/mL, 5 ng/mL, or a range defined by any two of the foregoing values of SEQ ID NOs: 1, 12-18, 30, 38-39, 44, 46, 57, 61, 69, 86, 91, 92 or 101) or (b) antigen-negative HLA-A*01:01⁺ target cells into which a nucleotide sequence encoding the mutated target has been introduced such that the target cell expresses the mutated target. Cells expressing the inventive TCRs may also secrete IFN-gamma. upon co-culture with antigen-negative HLA-A*01:01⁺ target cells pulsed with higher concentrations of mutated target peptide.

Alternatively or additionally, a TCR may be considered to have “antigenic specificity” for a mutated target if T cells expressing the TCR secrete at least twice as much IFN-gamma upon co-culture with (a) antigen-negative HLA-A*01:01⁺ target cells pulsed with a low concentration of mutated target peptide or (b) antigen-negative HLA-A*01:01⁺ target cells into which a nucleotide sequence encoding the mutated target has been introduced such that the target cell expresses the mutated target as compared to the amount of IFN-gamma expressed by a negative control. The negative control may be, for example, (i) T cells expressing the TCR, co-cultured with (a) antigen-negative HLA-A*01:01⁺ target cells pulsed with the same concentration of an irrelevant peptide (e.g., some other peptide with a different sequence from the mutated target peptide) or (b) antigen-negative HLA-A*011:01⁺ target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative HLA-A*01:01⁺ target cells pulsed with the same concentration of mutated target peptide or (b) antigen-negative HLA-A*01:01⁺ target cells into which a nucleotide sequence encoding the mutated target has been introduced such that the target cell expresses the mutated target. IFN-gamma secretion may be measured by methods known in the art such as, for example, enzyme-linked immunosorbent assay (ELISA).

Alternatively or additionally, a TCR may be considered to have “antigenic specificity” for a mutated target if at least twice as many of the numbers of T cells expressing the TCR secrete IFN-gamma upon co-culture with (a) antigen-negative HLA-A*01:01⁺ target cells pulsed with a low concentration of mutated target peptide or (b) antigen-negative HLA-A*01:01⁺ target cells into which a nucleotide sequence encoding the mutated target has been introduced such that the target cell expresses the mutated target as compared to the numbers of negative control T cells that secrete IFN-gamma. The concentration of peptide and the negative control may be as described herein with respect to other aspects of the invention. The numbers of cells secreting IFN-gamma may be measured by methods known in the art such as, for example, ELISPOT.

Methods of engineering T cells to express recombinant T cell receptors for cancer treatment are disclosed in Ping et al Protein Cell. 2018 March; 9(3): 254-266.

The invention provides a TCR comprising two polypeptides (i.e., polypeptide chains), such as an alpha (alpha) chain of a TCR, a beta chain of a TCR, a gamma (gamma) chain of a TCR, a delta (delta) chain of a TCR, or a combination thereof. The polypeptides of the inventive TCR can comprise any amino acid sequence, provided that the TCR has antigenic specificity for the mutated target, e.g., mutated NRAS.

In an embodiment of the invention, the TCR comprises two polypeptide chains, each of which comprises a variable region comprising a complementarity determining region (CDR)1, a CDR2, and a CDR3 of a TCR.

As well as CDRs, the TCRs disclosed herein also comprise V regions and J regions. Particular combinations of V and J regions are presented in Table 3, herein below.

The sequences of CDR3 regions of exemplary β chains of T cell receptors which may be used according to this aspect of the present invention are those set forth in SEQ ID NO: 200, 202, 204, 206, 208 or 210.

The sequences of CDR3 regions of exemplary α chains of T cell receptors which may be used according to this aspect of the present invention are those set forth in SEQ ID NOs: 199, 201, 203, 205, 207 or 209.

It will be appreciated that the sequences of the CDR3 regions may comprise at least one or even two amino acid substitutions and retain binding activity.

In one embodiment, the amino acid substitution is a conservative substitution.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a peptide having anti-bacterial properties.

The T cell populations may be genetically modified to express a T cell receptor that binds to at least one of the peptide epitopes having the sequences as set forth in SEQ ID NOs: 1 and 12-132 (e.g. T cell receptors having the CDR3 amino acid sequences as set forth in SEQ ID NOs: 199-210).

According to a particular embodiment, the TCR receptor comprises an alpha chain which comprises a CDR3 region as set forth in SEQ ID NO: 209 and a beta chain which comprises a CDR3 region as set forth in SEQ ID NO: 210.

According to a particular embodiment, the TCR receptor comprises an alpha chain which comprises a CDR3 region as set forth in SEQ ID NO: 199 and a beta chain which comprises a CDR3 region as set forth in SEQ ID NO: 200.

According to a particular embodiment, the TCR receptor comprises an alpha chain which comprises a CDR3 region as set forth in SEQ ID NO: 201 and a beta chain which comprises a CDR3 region as set forth in SEQ ID NO: 202.

According to a particular embodiment, the TCR receptor comprises an alpha chain which comprises a CDR3 region as set forth in SEQ ID NO: 205 and a beta chain which comprises a CDR3 region as set forth in SEQ ID NO: 204.

According to a particular embodiment, the TCR receptor comprises an alpha chain which comprises a CDR3 region as set forth in SEQ ID NO: 207 and a beta chain which comprises a CDR3 region as set forth in SEQ ID NO: 208.

Also contemplated are isolated antibodies and/or diabodies which are capable of binding to at least one of the peptide epitopes having the sequences as set forth in SEQ ID NOs: 1 and 12-132. The antibodies/diabodies may comprise at least one of the CDR sequences specified herein.

The TCRs (and antibodies) of the invention of the invention can comprise synthetic amino acids in place of one or more naturally-occurring amino acids. Such synthetic amino acids are known in the art, and include, for example, aminocyclohexane carboxylic acid, norleucine, alpha-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, beta-phenylserine beta-hydroxyphenylalanine, phenylglycine, alpha-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, alpha-aminocyclopentane carboxylic acid, alpha-aminocyclohexane carboxylic acid, alpha-aminocycloheptane carboxylic acid, .alpha.-(2-amino-2-norbornane)-carboxylic acid, alpha, gamma-diaminobutyric acid, alpha, beta-diaminopropionic acid, homophenylalanine, and alpha-tert-butylglycine.

The TCRs (and antibodies) of the invention (including functional variants thereof) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCRs (and antibodies) of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4.sup.th ed., Cold Spring Harbor Press, Cold Spring Harbor, N.Y. (2012). Alternatively, the TCRs, polypeptides, and/or proteins described herein (including functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive TCRs, polypeptides, and proteins can be synthetic, recombinant, isolated, and/or purified. Included in the scope of the invention are conjugates, e.g., bioconjugates, comprising any of the inventive TCRs, polypeptides, or proteins, nucleic acids, recombinant expression vectors, host cells, populations of host cells, and antibodies, or antigen binding portions thereof. Conjugates, as well as methods of synthesizing conjugates in general, are known in the art.

An embodiment of the invention provides a nucleic acid comprising a nucleotide sequence encoding any of the TCRs (or antibodies) described herein. “Nucleic acid,” as used herein, includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetyl cytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N.sup.6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acids of the invention can be incorporated into a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as lamdaGT10, lamdaGT11, lamdaZapII (Stratagene), lamdaEMBL4, and lamdaNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech). Preferably, the recombinant expression vector is a viral vector, e.g., a retroviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from Co1E, 2.mu. plasmid, .lamda., SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

The populations of tumor-reactive T cells expressing subject-specific TCRs or may be combined with a pharmaceutically acceptable carrier to obtain a pharmaceutical composition comprising a personalized cell population of tumor-reactive T cells. Preferably, the carrier is a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The T cells can be administered by any suitable route as known in the art. Preferably, the T cells are administered as an intra-arterial or intravenous infusion, which preferably lasts approximately 30-60 min. Other examples of routes of administration include intraperitoneal, intrathecal and intralymphatic. T cells may also be administered by injection. T cells may be introduced at the site of the tumor.

For purposes of the invention, the dose, e.g., number of cells in the inventive cell population expressing subject specific TCRs, administered should be sufficient to effect, e.g., a therapeutic or prophylactic response, in the subject over a reasonable time frame. For example, the number of cells should be sufficient to bind to a cancer antigen, or detect, treat or prevent cancer in a period of from about 2 hours or longer, e.g., 12 to 24 or more hours, from the time of administration. In certain embodiments, the time period could be even longer. The number of cells will be determined by, e.g., the efficacy of the particular cells and the condition of the subject (e.g., human), as well as the body weight of the subject (e.g., human) to be treated.

Many assays for determining an administered number of cells from the inventive cell population expressing subject specific TCRs are known in the art. For purposes of the invention, an assay, which comprises comparing the extent to which target cells are lysed or one or more cytokines such as, e.g., IFN-gamma and IL-2 are secreted upon administration of a given number of such cells to a subject, could be used to determine a starting number to be administered to a mammal. The extent to which target cells are lysed, or cytokines such as, e.g., IFN-gamma and IL-2 are secreted, upon administration of a certain number of cells, can be assayed by methods known in the art. Secretion of cytokines such as, e.g., IL-2, may also provide an indication of the quality (e.g., phenotype and/or effectiveness) of a cell preparation.

The number of the cells administered from the inventive cell population expressing subject specific TCRs may also be determined by the existence, nature and extent of any adverse side effects that might accompany the administration of a particular cell population.

The present invention further contemplates tetramers expressing the T cell epitopes disclosed herein (peptides having an amino acid sequences as set forth in SEQ ID NOs: 1 and 12-132). The tetramers can be used in a tetramer assay. The tetramers comprise the 4 copies of one of the peptides as set forth in SEQ ID NOs. 1 and 12-132, each peptide being presented by the appropriate MHC molecule as summarized in Table 1C. The tetramer is typically labeled with a fluorophore.

Any cell (e.g. E. coli) may be used to synthesize the light chain and a shortened version of the heavy chain that includes a biotin amino acid recognition tag. These MHC chains are biotinylated with the enzyme BirA and refolded with the antigenic peptide described herein. Fluorophore tagged streptavidin is added to the bioengineered MHC monomers, and the biotin-streptavidin interaction causes four MHC monomers to bind to the streptavidin and create a tetramer.

It is expected that during the life of a patent maturing from this application many relevant checkpoint inhibitors will be developed and the scope of the term checkpoint inhibitors is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Cell-Lines and TILs

The tumor cell-lines utilized in this study were collected from several sources. 17T and 135T tumor cells and TILs were collected from patients with metastatic melanoma and were established as described previously⁴¹. Whole-exome sequencing was obtained for 17T, as previously described⁴². Established TILs were expanded according to the Rapid Expansion Protocol (REP), as described previously⁴³

W6/32 hybridoma cells (HB95) and IVA12 hybridoma cells (HB145) were purchased from ATCC⁴⁴. Commercial tumor cell line SK-MEL-30 (ACC-151) was purchased from DSMZ⁴⁵. The EBV-transformed B-cells IHW01161, IHW01113 and IHW01070 were purchased from the IHWG Cell and DNA Bank³⁸. The hybridoma cells HB95 and HB145 were used to purify pan-HLA-I and pan-HLA-II antibodies for the preparation of the HLA affinity columns. All cell lines were tested regularly and were found negative for mycoplasma contamination (EZ-PCR Mycoplasma Kit, Biological Industries).

HLA-Typing of Tumor Tissue

HLA-typing of 17T cells was computationally extracted from whole-exome data using the PolySolver software⁴⁶. High-resolution, Sanger sequence-based typing of the HLA-I A locus was performed on genomic DNA extracted from the 135T cell line, using QIAGEN's DNeasy Blood and Tissue kit (Cat #69504) and GeneDx SBTexcellerator HLA-A kit (Cat #4100234). HLA-typing of the utilized commercial tumor cell lines was done using the seq2HLA software tool, as they appear in the “TRON cell line portal”⁴⁶′⁴⁷. Tumor cell-line MM121224 was previously HLA-typed by Prof. Mitch Levesque (University of Zurich Hospital, Zurich, Switzerland). HLA-typing of EBV-transformed B-cells was provided by the IHWG Cell and DNA Bank³⁸. High-resolution HLA-typing of 9176 patients, forming the TCGA pan-cancer cohort, was kindly provided by Prof. Hannah Carter⁴⁸.

NetMHCpan Predictions

The NetMHCpan 4.0 software package served to scan the landscape of RAS.Q61-derived peptides for ones predicted to bind common HLA alleles³⁰. 27-mer peptide variants flanking position 61 of the RAS family consensus C-terminal amino-acid sequence were constructed with alternating 61 position substitutions, representing both wild-type and common-mutant diversity. The Wild-type variant has a peptide sequence GETCLLDILDTAGQEEYSAMRDQYMRT (SEQ ID NO: 5). The Q61R variant has a peptide sequence GETCLLDILDTAGREEYSAMRDQYMRT (SEQ ID NO: 6). The Q61K variant has a peptide sequence GETCLLDILDTAGKEEYSAMRDQYMRT (SEQ ID NO: 7). The Q61L variant has a peptide sequence GETCLLDILDTAGLEEYSAMRDQYMRT (SEQ ID NO: 8). The Q61H variant has a peptide sequence GETCLLDILDTAGHEEYSAMRDQYMRT (SEQ ID NO: 9).

Based on HLA allele frequency in the pan-cancer TCGA cohort, the 15 most abundant alleles for each class-I loci were aggregated to form a list of 45 frequent alleles (Table 1A).

TABLE 1A

| | count of | |
|---|---|---|
| | patients | |
| | possessing | |
| Allele | allele | Frequency |
| A*02:01 | 3799 | 41.40 |
| A*01:01 | 2324 | 25.33 |
| A*03:01 | 2120 | 23.10 |
| A*24:02 | 1578 | 17.20 |
| A*11:01 | 1059 | 11.54 |
| A*68:01 | 605 | 6.59 |
| A*26:01 | 566 | 6.17 |
| A*32:01 | 561 | 6.11 |
| A*31:01 | 514 | 5.60 |
| A*29:02 | 463 | 5.05 |
| A*23:01 | 440 | 4.80 |
| A*25:01 | 423 | 4.61 |
| A*30:01 | 344 | 3.75 |
| A*33:03 | 257 | 2.80 |
| A*30:02 | 238 | 2.59 |
| B*07:02 | 1927 | 21.00 |
| B*08:01 | 1531 | 16.68 |
| B*44:02 | 1221 | 13.31 |
| B*35:01 | 1081 | 11.78 |
| B*51:01 | 924 | 10.07 |
| B*15:01 | 881 | 9.60 |
| B*18:01 | 851 | 9.27 |
| B*44:03 | 774 | 8.44 |
| B*40:01 | 753 | 8.21 |
| B*27:05 | 570 | 6.21 |
| B*57:01 | 497 | 5.42 |
| B*14:02 | 452 | 4.93 |
| B*13:02 | 405 | 4.41 |
| B*38:01 | 387 | 4.22 |
| B*35:03 | 288 | 3.14 |
| C*07:01 | 2391 | 26.06 |
| C*07:02 | 2192 | 23.89 |
| C*04:01 | 2029 | 22.11 |
| C*06:02 | 1493 | 16.27 |
| C*05:01 | 1264 | 13.78 |
| C*03:04 | 1145 | 12.48 |
| C*12:03 | 964 | 10.51 |
| C*03:03 | 802 | 8.74 |
| C*01:02 | 779 | 8.49 |
| C*02:02 | 742 | 8.09 |
| C*16:01 | 600 | 6.54 |
| C*08:02 | 577 | 6.29 |
| C*15:02 | 453 | 4.94 |
| C*07:04 | 286 | 3.12 |
| C*17:01 | 270 | 2.94 |

NetMHCpan 4.0 was executed with these 27-mer peptides and HLA allele lists as input, in FASTA mode, restricting to peptide lengths of 8-14 amino-acids. The output was filtered to retain only peptides spanning the 61 position. Peptides ranked (% Rank) at ≤0.5 were considered predicted strong binders. Peptides ranked at 0.5≤% Rank≤2 were considered predicted weak binders.

NetMHCpan 4.0 was executed with these 27-mer peptides and HLA allele lists as input, in FASTA mode, restricting to peptide lengths of 8-14 amino-acids. The output was filtered to retain only peptides spanning the 61 position. Peptides ranked (% Rank) at ≤0.5 were considered predicted strong binders. Peptides ranked at 0.5≤% Rank≤2 were considered predicted weak binders.

TCGA Analysis of RAS.61 and HLA Allele Frequencies

The data of TCGA provisional cohorts were downloaded via cBioportal, accumulating to a total of 8038 patients⁴⁹⁻⁵¹. Previously published high-resolution HLA class-I typing data of 9176 pan-cancer TCGA patients was obtained as described above²⁷. Patient mutation data was aggregated with HLA-typing data, resulting in an intersected database of 6840 patients in total, 364 of whom melanoma patients. Pan-cancer HLA frequencies were calculated. The frequency of N/K/H-RAS.61 mutations and their subtypes were calculated for both pan-cancer and melanoma only. The 15 most abundant HLA alleles for each class-I locus, i.e., A, B and C, were chosen for further analysis, resulting in 45 analyzed alleles in total. For each HLA allele, the total number of patients harboring an N/K/H-RAS.61 mutation was calculated both for pan-cancer and melanoma alone. Intersection frequencies were compared to expected frequencies under an independence assumption.

Structural Modeling of HLA-A*01:01 in Complex

[symbol]

Structures of HLA-A*01:01 complexed with RAS.61-derived peptides were modelled using a crystallographic complex featuring HLA-A*01:01 in complex with an ALK tyrosine kinase receptor decapeptide (PDB: 6at9)³⁴. The crystallographic bound peptide was manually mutated to yield the ILDTAGKEEY (SEQ ID NO: 1) and ILDTAGQEEY (SEQ ID NO: 2) peptides in complex with the HLA receptor. The HLA structure was truncated to the peptide-binding domain (chain A, residues 1 to 180). The resulting peptide-HLA structures were used as starting conformations for peptide docking and for molecule dynamics simulation.

Peptide docking was performed using the freely accessible web server interfaces FlexPepDock³⁵, ClusPro³⁶ and DINC³⁷. Molecular dynamics simulations were performed using GROMACS version 2018.3.⁵² with a GROMOS 54a7 united atom force field⁵³. The complex was placed in a rhombic dodecahedral box with a minimum distance of 10 Å between the solute and box wall, and solvated by SPC water. The system's charge was neutralized by the addition of 5 Na⁺ counter ions. Steric clashes were removed by minimization, conducted using the steepest descent algorithm for a maximum of 5,000 steps. The system was equilibrated at constant volume and temperature (NVT ensemble) with all protein and peptide heavy atoms restrained for 100 ps at 10° K, followed by further equilibration without restraints for another 100 ps at 300° K. The system's pressure was equilibrated by simulation under constant atmospheric pressure (NPT ensemble) for 300 ps at 300° K. Positional restraints were applied to protein residues during all equilibration steps using the LINCS algorithm⁵⁴. The final coordinates resulting from equilibration were used to commence five independent production runs for both (ILDTAGKEEY (SEQ ID NO: 1) and ILDTAGQEEY (SEQ ID NO: 2)) systems, each conducted for 500 ns in the NPT ensemble. The temperature was held constant at 300° K using the velocity rescaling thermostat⁵⁵ coupled with a time constant of 0.1 ps, and the system's pressure was kept constant at 1 bar using the Parrinello-Rahman barostat⁵⁶ coupled with a time constant of 2 ps. A timestep of 2fs was used to integrate the motions of the system. Long-range electrostatics were calculated using the Particle Mesh Ewald⁵⁷ method, while short-range cutoffs were set to 1.0 nm for both the vdW and Coulomb interactions.

To focus on bound conformations, only conformations in which the RAS peptide's N- and C-termini were within 7 Å of the HLA B- and F-pockets, respectively, were analyzed. Distances were measured between the following atoms: HLA Tyr171 sidechain hydroxyl oxygen (OH) and peptide Ile1 backbone amide nitrogen (N), and between HLA Tyr123 sidechain phenolic carbon (CZ) and peptide Tyr10 sidechain gamma carbon (CG). Molecular structures were visualized using PyMOL version 1.3⁵⁸. The conformations from docking and from bound simulation frames were clustered by HLA-peptide hydrogen-bonding interactions. Hydrogen bonds were detected using the Wernet-Nilsson criteria⁵⁹ as implemented in MDTraj⁶⁰ version 1.9.2. Cluster centroids were identified as simulation frames for which the corresponding hydrogen bonding fingerprint possessed the lowest Manhattan/Cityblock distance to all cluster members.

cDNA Sequencing

Total RNA was extracted from melanoma cell lines 17T, 135T, SK-MEL-30 and MM121224 following the manufacturer's protocol using the RNeasy Mini Kit (Cat#74104, QIAGEN), and eluted in 30p1 diethylpyrocarbonate (DEPC)-treated distilled H2O. A total of 500 ng RNA was used for single-strand complementary DNA (cDNA) synthesis using the iScript Reverse Transcription Super mix for RT-qPCR kit (Cat#1708841, Biorad) as per the manufacturer's protocol. The NRAS region containing position 61 was amplified by PCR using forward primer (5′ TTGGAGCAGGTGGTGTTGGG-3′(SEQ ID NO: 3)) and reverse primer (5′—GTATCAACTGTCCTTGTTGGC 3′(SEQ ID NO: 4)). 2 μl of cDNA were taken for the PCR reaction, mixed with 2×KAPA HIFI (Cat# KM2605 KAPA Biosystems) to a final volume of 25 μl, using a standard PCR program with the following parameters: one cycle at 95° C. for 3 min; 35 20 sec cycles of 98° C.; annealing temperature of 58° C. for 30 sec; and one cycle at 72° C. for 1 min. The PCR products were separated on a 1% agarose gel and then purified by Wizard SV Gel and PCR Clean-Up System (Cat# A9281, Promega), followed by Sanger sequencing using a 3730 DNA Analyzer (ABI). The sequencing primers were the same as the PCR primers. The sequencing results were analyzed using the SnapGene software (Version 4.3.2).

Production and Purification of Membrane HLA Molecules:

Cell pellets consisting of 2×10⁸ cells each were collected and lysed on ice using a lysis buffer containing 0.25% sodium deoxycholate, 0.2 mM iodoacetamide, 1 mM EDTA, 1:200 Protease Inhibitors Cocktail (Sigma-Aldrich, P8340), 1 mM PMSF and 1% octyl-b-D glucopyranoside in PBS. Samples were then incubated at 4° C. for 1 h. The lysates were cleared by centrifugation at 48,000 g for 60 min at 4° C. and then passed through a pre-clearing column containing Protein-A Sepharose beads.

HLA-I molecules were immunoaffinity purified from cleared lysate with the pan-HLA-I antibody (W6/32 antibody purified from HB95 hybridoma cells) covalently bound to Protein-A Sepharose beads or to Amino-Link beads (Thermo-Fisher Scientific, as reported previously)^{61,62.} Affinity columns were washed first with 10 column volumes of 400 mM NaCl, 20 mM Tris-HCl and then with 10 volumes of 20 mM Tris-HCl, pH 8.0. The HLA peptides and HLA molecules were then eluted with 1% trifluoracetic acid, followed by separation of the peptides from the proteins by binding the eluted fraction to disposable reversed-phase C18 columns (Harvard Apparatus). Elution of the peptides was done with 30% acetonitrile in 0.1% trifluoracetic acid⁶¹. The eluted peptides were then cleaned using C18 stage tips as described previously⁶³.

Identification of Eluted HLA Peptides

Liquid Chromatography

Cell lines 17T, SK-MEL-30 and MM121224: The HLA peptides were dried by vacuum centrifugation, solubilized with 0.1% formic acid, and resolved with a 7-40% acetonitrile gradient with 0.1% formic acid for 180 min and 0.15 μL/min on a capillary column pressure-packed with Reprosil C18-Aqua (Dr. Maisch, GmbH, Ammerbuch-Entringen, Germany) as previously described⁶⁴. For cell lines 17T, SK-MEL-30 and MM121224, chromatography was performed with the UltiMate 3000 RSLCnano-capillary UHPLC system (Thermo Fisher Scientific), which was coupled by electrospray to tandem mass spectrometry on Q-Exactive-Plus (Thermo Fisher Scientific). HLA peptides were eluted over 2h with a linear gradient from 5% to 28% acetonitrile with 0.1% formic acid at a flow rate of 0.15 μl/min.

Cell line 135T: ULC/MS grade solvents were used for all chromatographic steps. Each sample was solubilized in 12 μL 97:3 waster: acetonitrile with 0.1% formic acid. Samples were loaded using split-less nano-Ultra Performance Liquid Chromatography (10 kpsi nanoAcquity; Waters, Milford, Mass., USA). The mobile phase was: A) H₂O+0.1% formic acid and B) acetonitrile+0.1% formic acid. Desalting of the samples was performed online using a reversed-phase Symmetry C18 trapping column (180 μm internal diameter, 20 mm length, 5 μm particle size; Waters). The peptides were then separated using a T3 HSS nano-column (75 μm internal diameter, 250 mm length, 1.8 μm particle size; Waters) at 0.35 μL/min. HLA peptides were eluted from the column into the mass spectrometer using the following gradient: 5% to 28% B in 120 min, 28% to 35% B in 15 min, 35% to 95% in 15 min, maintained at 95% for 10 min and then back to initial conditions.

Mass Spectrometry

For the 17T cell line, the experiment was run in discovery mode. Cell-lines SK-MEL-30, MM121224 and 135T and 17T were analyzed in an absolute targeted mass spectrometry, looking for ILDTAGKEEY (SEQ ID NO: 1) specifically, and utilizing heavy-peptide spike-in, enabling also for peptide quantification). Synthetic heavy-isotope-labeled ILDTAGKEEY (SEQ ID NO: 1), with heavy lysine (12C6; 15N2) incorporated, was purchased in >95% purity from JPT.

17T discovery mode: Data was acquired using a data-dependent “top-10” method, fragmenting the peptides by higher-energy collisional dissociation. The full-scan MS spectra were acquired at a resolution of 70,000 at 200 m/z with a target value of 3×10⁶ ions. Ions were accumulated to an automatic gain control (AGC) target value of 10⁵ with a maximum injection time of generally 100 msec. The peptide match option was set to Preferred. The normalized collision energy was set to 25% and the MS/MS resolution was 17,500 at 200 m/z. Fragmented m/z values were dynamically excluded from further selection for 20 sec. MS data were analyzed using MaxQuant (version 1.5.8.3)⁶⁰ with FDR 0.05. The peptide identifications were based on the human section of the UniProt database⁶⁵ (April 2017) and a customized reference database that contained the mutated sequences identified for 17T by WES.

SK-MEL-30, MM121224 absolute targeted mode: 0.1 pmol heavy peptide was added to the peptidome sample injected into the mass-spectrometer. Analysis was then performed using the PRM method. An inclusion list was imported into the method for MS/MS acquisitions. The instrument switched between full MS and MS/MS acquisitions to fragment the ions in the inclusion list. Full-scan MS spectra were acquired at a resolution of 70,000, with a mass-to-charge ratio (m/z) of 350-1,400 AMU. Fragmented masses were accumulated to an AGC target value of 10⁵ with a maximum injection time of 400 msec and 1.8 m/z window. Analysis again utilized the MaxQuant software (version 1.5.8.3)⁶⁰ with the Andromeda search engine⁶⁶. The ILDTAGKEEY (SEQ ID NO: 1) neo-antigen was manually added to the human UniProt database (April 2017). The following parameters were used: precursor ion mass and fragment mass tolerance of 20 ppm, false discovery rate (FDR) of 0.05 for SK-MEL-30 and 0.3 for MM121224, and variable modification of oxidation (Met), acetylation (protein N-terminus) and heavy Lysine (12C6; 15N2).

135T, 17T Absolute Targeted Mode:

The nanoUPLC was coupled online through a nanoESI emitter (10 μm tip; New Objective; Woburn, Mass., USA) to a quadrupole orbitrap mass spectrometer (Q Exactive Plus, Thermo Scientific) using a Flexion nanospray apparatus (Proxeon). Data was acquired in Parallel Reaction Monitoring (PRM) with one MS1 scan for every 10 PRM scans. MS1 scan range was set to 300-1800 m/z, resolution of 70,000, AGC of 3e6 and maximum injection time was set to 120 msec. The PRM channels were acquired at 35,000 resolution, maximum injection time of 200 msec, AGC of 2e5, NCE of 27 and isolation of 1.7 m/z.

Peptide Quantification:

Raw PRM data was imported into Skyline. Absolute quantification was obtained by summing extracted ion chromatograms of all fragment ions per peptide and exporting the ratio of total signal of the native peptide versus the heavy labeled internal standard that was spiked into the sample, multiplied by the amount of internal standard.

Analysis of T-Cell Reactivity by IFN-γ Release Assay

IFNγ's release from TIL, as measured in an enzyme-linked immunosorbent assay (ELISA), was used to quantify reactivity. Synthetic pure (>95% purity) mutant (ILDTAGKEEY (SEQ ID NO: 1)) and wild-type (ILDTAGQEEY (SEQ ID NO: 2)) peptides were purchased from GenScript and dissolved in DMSO.

EBV-transformed B-cells bearing HLA allele A*01:01 were used for peptide pulsing. A B-cell suspension at 1×10⁶ cells/ml was incubated with the peptide of choice, at the desired concentration (0.001-10 μg/ml), for 4 h in a 37° C., 5% CO₂, humidified incubator. The DMSO volume was kept at 1% in all samples. For the no-peptide control, DMSO devoid of peptides was added. The B-cells were washed in PBS three times before proceeding to the co-incubation with TILs. TILs were co-cultured with either cognate melanoma or EBV-transformed B-cells at a 1:1 ratio (10⁵-2×10⁶ cells) and incubated overnight in a 37° C., 5% CO₂, humidified incubator. The soluble IFNγ secreted from TILs was quantified from the co-culture supernatant using Biolegend Human IFN-γ ELISA MAX Deluxe (Cat#430106). All experiments were conducted in biological triplicates.

Fluorescence-Based In Vitro Killing Assay:

In the killing assay, loss of fluorescent content was used to quantify target cell death^{13,62}. Melanoma cell lines were infected to stably express GFP. The GFP-expressing lentiviral vector pCDH-CMV-MCS-EFlα-GreenPuro (System Biosciences, Cat# CD513B-1) was packaged with psPAX and pMD2.G helper plasmids (Addgene) to form viral particles. The plasmids were co-transfected into HEK293T cells seeded at 3×10⁶ per 10-cm plates using Turbofect (Thermo-fisher scientific, Cat # R0532) as described by the manufacturer. Virus-containing media was harvested 72 h after transfection, filtered and aliquoted. GFP-expressing cells were selected for 48 h after infection with 3 mg/ml and 2 mg/ml puromycin for 17T and 135T cells, respectively. The GFP-expressing melanoma cells, i.e., the target cells, were plated in 48-well plates with a puromycin devoid, 10% FCS supplemented, RPMI-1640 growth medium and incubated overnight at 37° C. and 5% CO₂ in a humidified incubator to form an attached monolayer of cells at 100% confluence. 1.5×10⁵ and 0.5×10⁵ cells per well were plated for 17T, and 135T, respectively. Cognate tetramer-positive sorted TILs were then added at effector to target (E:T) ratios ranging from 0:1 to 4:1. Co-incubation plates were incubated at 37° C. and 5% CO₂ in a humidified incubator. The highest E:T condition in each experiment was monitored periodically under light microscopy for melanoma killing. The experiment was terminated upon perceived total melanoma killing. For 17T, the duration of co-culture was 16 h, whereas for 135T, the experiment lasted 24 h. After incubation, non-adherent TILs and dead target cells were washed away with PBS. The fluorescence of the remaining live target cells was quantified using a Typhoon-9410 laser flatbed scanner (GE Healthcare, USA). The fluorescence reading was focused 3 mm above the plate surface. The percentage of specific lysis was calculated as 100×(C−X)/C, where C is the fluorescence in the TIL-free condition and X is the fluorescence in the presence of TILs. All experiments were performed in biological triplicates.

Flow Cytometry Analysis and Fluorescence-Activated Cell Sorting

TILs were stained with either the HLA-A*01:01/ILDTAGKEEY (SEQ ID NO: 1) tetramer (NIH Tetramer Facility) or anti-4-1BB antibody (309809, Biolegend). Staining for 4-1BB was done on rested TILs, to gauge baseline levels of activation, or after co-culture with cognate melanoma for 16-20 h, at a 1:1 ratio, 37° C. and 5% CO₂ in a humidified incubator. Tetramer staining was conducted on rested TILs. The BD LSR II (BD Biosciences) was used for flow cytometry, while the BD FACSAria III Cell Sorter (BD Biosciences) was used for fluorescence-activated cell sorting. Size and granularity measures served to gate on viable, singlet TILs. The TILs were further gated to distinguish neo-antigen specific or activated subpopulations, based on tetramer or anti-4-1BB staining, respectively. The sorting experiments gated on positive and negative sub-populations without overlap. After sorting, TILs were rested for 24 h before being used in downstream reactivity assays.

TCR Sequencing

TCR library preparation was prepared on sorted TILs, as was previously described⁶⁸. In short, RNA was extracted from TIL pools and treated with DNase (RNeasy Micro kit (QIAGEN), RQ1 RNase free DNase (Promega)). Reverse transcription was then performed using primers directed at the constant regions of the TC_{α/β} chains (SuperScript III (Invitrogen)). Single stranded oligonucleotides consisting of both a universal primer region and a unique molecular identifier (UMI) were ligated onto the 3′ end of the TCR cDNA transcripts (T4 RNA ligase). Over three consecutive PCR steps, the library was then adequately amplified and split into α and β chain pools. Libraries were sequenced with 300 cycles on the NextSeq Illumina platform and were processed using an in-house pipeline. Mainly, reads were: (1) clustered according to UMI, for accurate frequency evaluation; (2) annotated for V and J germline gene segments according to the IMGT predetermined library⁶⁹; and (3) determined for their CDR3 sequence at both the nucleotide and amino-acid level. The annotated output consisted of separate collections of α and β chains, and was further filtered to exclude non-productive and singleton sequences. Experiments were conducted in biological duplicates with 0.5×10⁶ cells collected for each replicate. Chain frequencies were averaged over duplicates.

Single-Cell RNA and TCR Sequencing of CD8+ 17TIL

17TIL and the cognate 17T melanoma cell-line were plated in 1:1 ratio at 4×106 cells per well in a 24-well tissue culture plate. After co-incubation overnight, the cells were washed, stained with the HLA-A*01:01/ILDTAGKEEY (SEQ ID NO: 1) tetramer (1:50) and then sorted into tetramer-positive and tetramer-negative fractions, as described above. Immediately after sorting, cells were washed and resuspended in PBS 0.04% BSA, strained using a 40 μm mesh (Corning, #431750), counted using trypan blue staining, and adjusted to 1000 live cells/μ1. Single-cell suspensions were loaded onto the Chromium Controller (10× Genomics) for droplet formation with targeted cell recovery aimed at 4000 cells for each sample. Single-cell RNAseq and TCRseq libraries were prepared using the Chromium Single Cell 5′ Reagent Kit and Single Cell V(D)J Kit (10× Genomics), respectively, according to manufacturer's protocol. Samples were sequenced on the NextSeq 11lumina platform with 26-bp read 1, 8-bp i7 index and 58-bp read 2 for gene-expression libraries, and on the miSeq with 150-bp paired end reads for TCR libraries. The Cell Ranger software (10× Genomics, version 3.0.0) was used for demultiplexing, initial quality assessment, alignment and quantification. Samples were aligned to the GRCh38 human genome assembly. In total, 4511 cells were recovered for the tetramer-positive fraction, with a median of 1291 sampled genes per cell. For the tetramer-negative fraction 4165 cells were recovered, with a median of 1790 genes per cell. Count matrices were generated using the count function with default settings, and were loaded onto the R package Seurat (version 2.3.4) for downstream analysis. Preliminary data inspection suggested non-specific tetramer staining of CD8+ T-cells at 1:50, with 96% of the annotated CD8 cells, i.e. cells where at least one of the genes CD8A/CD8B was detected, residing in the tetramer-positive fraction. We therefore decided to focus our analyses on the subpopulation of sequenced CD8+ TIL. To this end, we combined the samples in-silico, and retained only CD8 annotated cells for further analysis. For quality control, cells with high mitochondrial content (over 20% of total mRNA) or deviant UMI counts (bottom and top 5% of the sample's UMI distribution), were discarded. Unsupervised clustering was employed to gain insight into the functional states of CD8 cells in our dataset. Reads relating to the V(D)J genes of the TCR α and β chains were filtered out of the RNAseq data before clustering, to eliminate any non-functional clone bias. The data was further normalized and scaled using default package parameters, regressing out the number of UMIs, and the percentage of mitochondrial gene expression. For principal component analysis (PCA), 622 variable genes were identified using the FindVariableGenes function. Statistically significant principal component values were identified by means of the RunPCA command. Using an elbow plot, a cut-off of 15 leading dimensions was chosen for subsequent analysis. Clusters were determined using the shared nearest neighbor procedure, implemented in Seurat's FindClusters function, using k=8 and resolution=0.3. 2-dimensional t-SNE maps were generated for ease of visual inspection (RunTSNE command).

CDR3 sequences and V(D)J annotations were assigned by Cell Ranger's vdj command using the same IMGT reference database as in bulk TCR sequencing experiments. Cells with paired TCR annotations, i.e. one detected α chain and one detected β chain, were grouped into clones. However, preliminary analysis revealed an abundance of cells for which a single TCR chain was detected (674 singleton TCRβ and 90 singleton TCRα cells). Going back to gene-expression data, we observed that these cells with single-chain TCR annotations tend to cluster with, and share the same V region genes as their cognate paired-TCR, clone-defining, cells (see FIG. 15A-J). We therefore broadened our clone definition to incorporate these cells as well, and clones of interest were expanded by aggregation of cells with matching single chain annotation (a or (3).

Predefined gene expression signatures were used to assess clusters and clones on scales of cytotoxicity, exhaustion and proliferation. Cytotoxicity (NKG7, CCL4, CST7, PRF1, GZMA, GZMB, IFNG, CCL3) and exhaustion (PD1, TIGIT, LAG3, TIM3, CTLA4) markers, as well as a G2/M signature based on 54 genes. Marker lists' average expression levels for each cluster were calculated using Seurat's AddModuleScore function.

Electroporation of in-vitro transcribed mRNA into donor peripheral blood mononuclear cells: Electroporation of in-vitro transcribed (IVT) mRNA was utilized for transient TCR expression in primary T-cells, with slight modifications to the previously described procedure. V(D)J sequences of leading tetramer-enriched TCRα and TCR(3 chains were reconstructed from TCR sequencing data (see Table 1B).

TABLE 1B

| TCR name | Chain name | | CDR3 | V region | J region |
|---|---|---|---|---|---|
| N135.1 | NA135.1 | alpha | 209 | TRAV19 | TRAJ39 |
| | NB135.1 | beta | 210 | TRBV6-1 | TRBJ1-1 |
| N17.1 | NA17.1 | alpha | 199 | TRAV17 | TRAJ49 |
| | NB17.1 | beta | 200 | TRBV27 | TRBJ2-5 |
| N17.2 | NA17.2 | alpha | 201 | TRAV5 | TRAJ9 |
| | NB17.2 | beta | 202 | TRBV6-6 | TRBJ2-7 |
| N17.3 | NA17.4 | alpha | 205 | TRAV19 | TRAJ17 |
| | NB17.3 | beta | 204 | TRBV6-1 | TRBJ1-1 |
| N17.5 | NA17.6 | alpha | 207 | TRAV19 | TRAJ17 |
| | NB17.5 | beta | 208 | TRBV6-1 | TRBJ1-1 |

TCRα/TCRβ pairings were deduced from single-cell data or chain frequencies in bulk TCR sequencing data. The TCR variable regions were fused to murine constant domains, as previously described, to increase cell surface expression of the desired α/β pairings. Full TCRα and TCRβ codon optimized sequences were purchased as synthetic double stranded DNA (Genscript or Twist bioscience). Each chain was individually cloned into the pGEM-4A/64A plasmid, using the NcoI/XbaI and NotI restriction sites (NEB numbers). Cloned plasmids were linearized with the restriction enzyme SpeI-HF (NEB, #R3133L). mRNAs were generated from linearized pGEM-4A/64A plasmids by IVT using “T7-Scribe Standard RNA IVT Kit” (Cellscript, #C-AS2607), and then further purified using “RNA clean-up and concentration kit” (Norgen biotek, #23600). Healthy donor whole blood preparations were purchased from Israel's national EMS organization (‘Magen David Adom’). PBMC were separated from whole blood by centrifugation on a Ficoll-Paque cushion (GE healthcare, 17-1440-03), and frozen in 10×10⁶ cells aliquots. For each electroporation experiment, PBMC were thawed and resuspended at a concentration of 1×10⁶ cells/mL in prewarmed TIL medium supplemented with 50 ng/mL OKT-3 (LEAF anti-human CD3mAb, Biolegend, #317304), and 300 IU/mL IL-2 (Proleukin, Clinigen). Cells were plated in tissue-culture treated 24-well plates at 2 mL per well, and were kept in culture for 3-7 days prior to electroporation. IL-2 was supplemented every three days, cells were subcultured and medium was replenished, as needed to maintain the cells at an approximate density of 1×10⁶ cells/mL. For electroporation, the PBMC (90%+T-lymphocytes after culturing with OKT-3 and IL-2) were washed in Opti-MEM (Gibco, #11058021), then resuspended in Opti-MEM at 2×10⁷ cells/mL. 100 μL cell suspension aliquots were mixed with mRNA preparations at 5 μg per transcript, i.e. TCRα and TCRβ pairs were mixed together (5 μg each) into the same cell suspension aliquot to test their pairing. Cell/mRNA suspensions were transferred into 2 mm cuvettes (BTX, #45-0141) and then electroporated using the ECM 830 electroporator (BTX, San Diego, Calif.) using the following conditions: mode=LV, voltage=0400V, pulse length=0500 μs. Following electroporation, cells were transferred to 24-well plates containing fresh medium and cultured at 37° C. Electroporated cells were rested for at least two hours before use in downstream assays. To evaluate surface expression of the desired TCR chains, cells were stained for mouse TCRβ constant region (antibody clone H57-597, Biolegend) at 6-30 hours post electroporation and were analyzed in flow cytometry.

Results

Data-Driven Recurrent Candidate Mutation Selection Based on Patient Data and Binding Predictions

The present inventors chose to focus their discovery efforts on NRAS, the second most highly mutated oncogene in melanoma and, specifically, on position 61, which is the most recurrently mutated position of the protein. Glutamine 61 lies within an 86-long N-terminal sequence that is shared by all main isoforms of the RAS family proto-oncogenes, including NRAS, KRAS and HRAS. Derived neo-peptides may, therefore, apply beyond NRAS also to other RAS.61-mutated tumors.

To qualify as an interesting hotspot neo-antigen candidate, the combined HLA-allele/RAS.61 mutation frequency should be harbored by a high number of cancer patients²⁷. Therefore, the TCGA pan-cancer cohort was utilized aggregating both the mutation data and HLA class-I allotypes of 6840 cancer patients, to explore the potential of different HLA alleles to form recurrent neo-antigen with the RAS.61 mutation. Owing to the high recurrence rate of RAS.61 mutations specifically in melanoma, the present inventors focused on the TCGA melanoma cohort, which consists of 364 individuals.

First, they calculated the frequency of HLA class-I alleles in the pan-cancer cohort, and compiled a list of 45 highly frequent alleles, consisting of the 15 top alleles of each locus, for further analysis (Table 1A). Allele frequencies were then intersected with RAS.61 mutation frequencies. No significant frequency skews were observed for RAS.61 mutant melanoma. Therefore, the most frequent HLA alleles remained the most interesting candidates for further exploration (FIG. 5A-B).

To further assess the potential of frequent HLA alleles in binding RAS.61-derived neo-antigens, NetMHCpan was used, the most commonly used prediction algorithm for peptide/HLA binding³⁰. In the literature, NetMHCpan is known to produce high sensitivity/low specificity predictions³¹. While an unbiased approach, such as HLA-peptidomics, may still uncover non-predicted binders, “looking under the streetlight” of binding predictions may serve as a reasonable starting point for HLA class-I recurrent neo-antigen binder discovery.

The present inventors queried the NetMHCpan algorithm (version 4.0) for RAS.61 mutation-bearing peptides of length 8-14 that are predicted to bind the list of common HLA class-I alleles. Considering all the four prevalent position 61 amino-acid substitutions, i.e., arginine, lysine, leucine and histidine, a total of 70 unique peptides were predicted to bind, including 13 predicted strong binders (% Rank≤0.5), and 57 predicted weak binders (0.5<% Rank≤2) (Table 1C-D).

TABLE 1C

| | | | | | Binding |
|---|---|---|---|---|---|
| | binding HLA | RAS | peptide | % | status |
| peptide sequence | allele | variant | length | Rank | (SB/WB) |
| ILDTAGLEEY (SEQ ID | HLA-A*01:01 | Q61L | 10 | 0.0564 | SB |
| NO: 12) | | | | | |
| ILDTAGHEEY (SEQ ID | HLA-A*01:01 | Q61H | 10 | 0.0922 | SB |
| NO: 13) | | | | | |
| LLDILDTAGHEEY | HLA-A*01:01 | Q61H | 13 | 0.1401 | SB |
| (SEQ ID NO: 14) | | | | | |
| LLDILDTAGLEEY | HLA-A*01:01 | Q61L | 13 | 0.155 | SB |
| (SEQ ID NO: 15) | | | | | |
| ILDTAGREEY | HLA-A*01:01 | Q61R | 10 | 0.1591 | SB |
| (SEQ ID NO: 16) | | | | | |
| LLDILDTAGREEY | HLA-A*01:01 | Q61R | 13 | 0.16 | SB |
| (SEQ ID NO: 17) | | | | | |
| LLDILDTAGKEEY | HLA-A*01:01 | Q61K | 13 | 0.1636 | SB |
| (SEQ ID NO: 18) | | | | | |
| ILDTAGKEEY - SEQ | HLA-A*01:01 | Q61K | 10 | 0.1676 | SB |
| ID NO: 1 | | | | | |
| DTAGHEEYSAM | HLA-A*26:01 | Q61H | 11 | 0.1737 | SB |
| (SEQ ID NO: 19) | | | | | |
| DTAGLEEYSAM | HLA-A*26:01 | Q61L | 11 | 0.2 | SB |
| (SEQ ID NO: 20) | | | | | |
| DTAGREEYSAM | HLA-A*26:01 | Q61R | 11 | 0.2672 | SB |
| (SEQ ID NO: 21) | | | | | |
| DTAGHEEYSAM | HLA-A*25:01 | Q61H | 11 | 0.2928 | SB |
| (SEQ ID NO: 22) | | | | | |
| DTAGLEEYSAM | HLA-A*25:01 | Q61L | 11 | 0.2948 | SB |
| (SEQ ID NO: 23) | | | | | |
| DTAGKEEYSAM | HLA-A*26:01 | Q61K | 11 | 0.2996 | SB |
| (SEQ ID NO: 24) | | | | | |
| LLDILDTAGL | HLA-C*05:01 | Q61L | 10 | 0.3732 | SB |
| (SEQ ID NO: 25) | | | | | |
| DTAGREEYSAM | HLA-A*25:01 | Q61R | 11 | 0.435 | SB |
| (SEQ ID NO: 26) | | | | | |
| LLDILDTAGL | HLA-C*08:02 | Q61L | 10 | 0.4646 | SB |
| (SEQ ID NO: 27) | | | | | |
| DTAGKEEYSAM | HLA-A*25:01 | Q61K | 11 | 0.4842 | SB |
| (SEQ ID NO: 28) | | | | | |
| DILDTAGLEEY | HLA-A*26:01 | Q61L | 11 | 0.5077 | WB |
| (SEQ ID NO: 29) | | | | | |
| DILDTAGLEEY | HLA-A*01:01 | Q61L | 11 | 0.565 | WB |
| (SEQ ID NO: 30) | | | | | |
| DTAGLEEY | HLA-A*26:01 | Q61L | 8 | 0.5942 | WB |
| (SEQ ID NO: 31) | | | | | |
| ETCLLDILDTAGR | HLA-A*68:01 | Q61R | 13 | 0.6012 | WB |
| (SEQ ID NO: 32) | | | | | |
| DTAGLEEYSAMR | HLA-A*68:01 | Q61L | 12 | 0.6288 | WB |
| (SEQ ID NO: 33) | | | | | |
| GETCLLDILDTAGL | HLA-B*40:01 | Q61L | 14 | 0.6729 | WB |
| (SEQ ID NO: 34) | | | | | |
| ILDTAGLEEYSAM | HLA-C*05:01 | Q61L | 13 | 0.6948 | WB |
| (SEQ ID NO: 35) | | | | | |
| REEYSAMRDQY | HLA-B*44:03 | Q61R | 11 | 0.712 | WB |
| (SEQ ID NO: 36) | | | | | |
| KEEYSAMRDQY | HLA-B*44:03 | Q61K | 11 | 0.7359 | WB |
| (SEQ ID NO: 37) | | | | | |
| CLLDILDTAGHEEY | HLA-A*01:01 | Q61H | 14 | 0.7455 | WB |
| (SEQ ID NO: 38) | | | | | |
| CLLDILDTAGLEEY | HLA-A*01:01 | Q61L | 14 | 0.7457 | WB |
| (SEQ ID NO: 39) | | | | | |
| HEEYSAMRDQY | HLA-B*44:03 | Q61H | 11 | 0.7591 | WB |
| (SEQ ID NO: 40) | | | | | |
| DTAGHEEYSAMR | HLA-A*68:01 | Q61H | 12 | 0.7991 | WB |
| (SEQ ID NO: 41) | | | | | |
| DILDTAGREEY | HLA-A*26:01 | Q61R | 11 | 0.8055 | WB |
| (SEQ ID NO: 42) | | | | | |
| LEEYSAMRDQY | HLA-B*44:03 | Q61L | 11 | 0.8138 | WB |
| (SEQ ID NO: 43) | | | | | |
| DILDTAGHEEY | HLA-A*01:01 | Q61H | 11 | 0.8152 | WB |
| (SEQ ID NO: 44) | | | | | |
| ILDTAGHEEYSAM | HLA-C*05:01 | Q61H | 13 | 0.825 | WB |
| (SEQ ID NO: 45) | | | | | |
| CLLDILDTAGREEY | HLA-A*01:01 | Q61R | 14 | 0.8271 | WB |
| (SEQ ID NO: 46) | | | | | |
| HEEYSAMRDQY | HLA-B*18:01 | Q61H | 11 | 0.832 | WB |
| (SEQ ID NO: 47) | | | | | |
| ILDTAGLEEY | HLA-A*29:02 | Q61L | 10 | 0.8336 | WB |
| (SEQ ID NO: 48) | | | | | |
| DILDTAGHEEY | HLA-A*26:01 | Q61H | 11 | 0.8376 | WB |
| (SEQ ID NO: 49) | | | | | |
| CLLDILDTAGKEEY | HLA-A*01:01 | Q61K | 14 | 0.8452 | WB |
| (SEQ ID NO: 50) | | | | | |
| DILDTAGKEEY | HLA-A*26:01 | Q61K | 11 | 0.867 | WB |
| (SEQ ID NO: 51) | | | | | |
| DTAGKEEYSAMR | HLA-A*68:01 | Q61K | 12 | 0.8698 | WB |
| (SEQ ID NO: 52) | | | | | |
| DTAGREEYSAMR | HLA-A*68:01 | Q61R | 12 | 0.8707 | WB |
| (SEQ ID NO: 53) | | | | | |
| LDILDTAGL | HLA-B*37:01 | Q61L | 9 | 0.8771 | WB |
| (SEQ ID NO: 54) | | | | | |
| REEYSAMRDQY | HLA-B*44:02 | Q61R | 11 | 0.913 | WB |
| (SEQ ID NO: 55) | | | | | |
| DTAGHEEY | HLA-A*26:01 | Q61H | 8 | 0.9161 | WB |
| (SEQ ID NO: 56) | | | | | |
| GLEEYSAMRDQY | HLA-A*01:01 | Q61L | 12 | 0.9427 | WB |
| (SEQ ID NO: 57) | | | | | |
| LEEYSAMRDQY | HLA-B*18:01 | Q61L | 11 | 0.9561 | WB |
| (SEQ ID NO: 58) | | | | | |
| ILDTAGHEEY | HLA-A*29:02 | Q61H | 10 | 0.9664 | WB |
| (SEQ ID NO: 59) | | | | | |
| KEEYSAMRDQY | HLA-B*44:02 | Q61K | 11 | 0.9705 | WB |
| (SEQ ID NO: 60) | | | | | |
| LDILDTAGLEEY | HLA-A*01:01 | Q61L | 12 | 0.9773 | WB |
| (SEQ ID NO: 61) | | | | | |
| LLDILDTAGK | HLA-A*03:01 | Q61K | 10 | 1.0274 | WB |
| (SEQ ID NO: 62) | | | | | |
| ETCLLDILDTAGK | HLA-A*68:01 | Q61K | 13 | 1.0365 | WB |
| (SEQ ID NO: 63) | | | | | |
| AGHEEYSAM | HLA-C*16:01 | Q61H | 9 | 1.0375 | WB |
| (SEQ ID NO: 64) | | | | | |
| HEEYSAMRDQY | HLA-B*44:02 | Q61H | 11 | 1.0413 | WB |
| (SEQ ID NO: 65) | | | | | |
| ILDTAGLEEYSAM | HLA-C*08:02 | Q61L | 13 | 1.0519 | WB |
| (SEQ ID NO: 66) | | | | | |
| ILDTAGREEYSAM | HLA-C*05:01 | Q61R | 13 | 1.0743 | WB |
| (SEQ ID NO: 67) | | | | | |
| ILDTAGKEEYSAM | HLA-C*05:01 | Q61K | 13 | 1.104 | WB |
| (SEQ ID NO: 68) | | | | | |
| ILDTAGLEEYS | HLA-A*01:01 | Q61L | 11 | 1.12 | WB |
| (SEQ ID NO: 69) | | | | | |
| ILDTAGHEEY | HLA-A*30:02 | Q61H | 10 | 1.1609 | WB |
| (SEQ ID NO: 70) | | | | | |
| ILDTAGHEEYSAM | HLA-C*08:02 | Q61H | 13 | 1.1742 | WB |
| (SEQ ID NO: 71) | | | | | |
| LEEYSAMRDQY | HLA-B*44:02 | Q61L | 11 | 1.1806 | WB |
| (SEQ ID NO: 72) | | | | | |
| AGLEEYSAM | HLA-C*03:03 | Q61L | 9 | 1.1839 | WB |
| (SEQ ID NO: 73) | | | | | |
| AGLEEYSAM | HLA-C*03:04 | Q61L | 9 | 1.1839 | WB |
| (SEQ ID NO: 74) | | | | | |
| ETCLLDILDTAGL | HLA-A*25:01 | Q61L | 13 | 1.1971 | WB |
| (SEQ ID NO: 75) | | | | | |
| AGHEEYSAM | HLA-C*03:03 | Q61H | 9 | 1.1979 | WB |
| (SEQ ID NO: 76) | | | | | |
| AGHEEYSAM | HLA-C*03:04 | Q61H | 9 | 1.1979 | WB |
| (SEQ ID NO: 77) | | | | | |
| REEYSAMRDQY | HLA-B*18:01 | Q61R | 11 | 1.2379 | WB |
| (SEQ ID NO: 78) | | | | | |
| LLDILDTAGL | HLA-A*02:01 | Q61L | 10 | 1.2399 | WB |
| (SEQ ID NO: 79) | | | | | |
| CLLDILDTAGL | HLA-A*02:01 | Q61L | 11 | 1.2596 | WB |
| (SEQ ID NO: 80) | | | | | |
| AGHEEYSAM | HLA-C*12:03 | Q61H | 9 | 1.2728 | WB |
| (SEQ ID NO: 81) | | | | | |
| DTAGLEEY | HLA-A*25:01 | Q61L | 8 | 1.2746 | WB |
| (SEQ ID NO: 82) | | | | | |
| AGLEEYSAM | HLA-C*16:01 | Q61L | 9 | 1.2903 | WB |
| (SEQ ID NO: 83) | | | | | |
| KEEYSAMRDQY | HLA-B*18:01 | Q61K | 11 | 1.2987 | WB |
| (SEQ ID NO: 84) | | | | | |
| AGREEYSAM | HLA-C*16:01 | Q61R | 9 | 1.3265 | WB |
| (SEQ ID NO: 85) | | | | | |
| DILDTAGREEY | HLA-A*01:01 | Q61R | 11 | 1.3332 | WB |
| (SEQ ID NO: 86) | | | | | |
| DTAGREEY | HLA-A*26:01 | Q61R | 8 | 1.3555 | WB |
| (SEQ ID NO: 87) | | | | | |
| ILDTAGREEYSAM | HLA-C*08:02 | Q61R | 13 | 1.3583 | WB |
| (SEQ ID NO: 88) | | | | | |
| ILDTAGLEEY | HLA-A*30:02 | Q61L | 10 | 1.3613 | WB |
| (SEQ ID NO: 89) | | | | | |
| ILDTAGKEEYSAM | HLA-C*08:02 | Q61K | 13 | 1.3703 | WB |
| (SEQ ID NO: 90) | | | | | |
| LDILDTAGHEEY | HLA-A*01:01 | Q61H | 12 | 1.3796 | WB |
| (SEQ ID NO: 91) | | | | | |
| DILDTAGKEEY | HLA-A*01:01 | Q61K | 11 | 1.4003 | WB |
| (SEQ ID NO: 92) | | | | | |
| AGREEYSAMR | HLA-A*31:01 | Q61R | 10 | 1.415 | WB |
| (SEQ ID NO: 93) | | | | | |
| ILDTAGREEY | HLA-A*30:02 | Q61R | 10 | 1.4159 | WB |
| (SEQ ID NO: 94) | | | | | |
| GREEYSAMRDQYM | HLA-B*27:05 | Q61R | 14 | 1.4995 | WB |
| (SEQ ID NO: 95) | | | | | |
| CLLDILDTAGK | HLA-A*03:01 | Q61K | 11 | 1.5047 | WB |
| (SEQ ID NO: 96) | | | | | |
| ILDTAGHEEY | HLA-C*05:01 | Q61H | 10 | 1.5198 | WB |
| (SEQ ID NO: 97) | | | | | |
| ILDTAGLEEY | HLA-C*05:01 | Q61L | 10 | 1.5428 | WB |
| (SEQ ID NO: 98) | | | | | |
| AGREEYSAM | HLA-C*12:03 | Q61R | 9 | 1.548 | WB |
| (SEQ ID NO: 99) | | | | | |
| ETCLLDILDTAGL | HLA-A*26:01 | Q61L | 13 | 1.5659 | WB |
| (SEQ ID NO: 100) | | | | | |
| ILDTAGHEEYS | HLA-A*01:01 | Q61H | 11 | 1.5862 | WB |
| (SEQ ID NO: 101) | | | | | |
| AGHEEYSAM | HLA-C*14:02 | Q61H | 9 | 1.5922 | WB |
| (SEQ ID NO: 102) | | | | | |
| LLDILDTAGHEEYS | HLA-A*01:01 | Q61H | 14 | 1.5996 | WB |
| (SEQ ID NO: 103) | | | | | |
| ETCLLDILDTAGR | HLA-A*33:03 | Q61R | 13 | 1.6356 | WB |
| (SEQ ID NO: 104) | | | | | |
| DTAGLEEYSAMR | HLA-A*33:03 | Q61L | 12 | 1.659 | WB |
| (SEQ ID NO: 105) | | | | | |
| DTAGREEYSAMR | HLA-A*33:03 | Q61R | 12 | 1.6668 | WB |
| (SEQ ID NO: 106) | | | | | |
| ILDTAGREEY | HLA-A*29:02 | Q61R | 10 | 1.6924 | WB |
| (SEQ ID NO: 107) | | | | | |
| REEYSAMRDQYM | HLA-B*40:01 | Q61R | 12 | 1.6932 | WB |
| (SEQ ID NO: 108) | | | | | |
| DILDTAGHEEYSAM | HLA-A*26:01 | Q61H | 14 | 1.7391 | WB |
| (SEQ ID NO: 109) | | | | | |
| LLDILDTAGLEEYS | HLA-A*01:01 | Q61L | 14 | 1.7415 | WB |
| (SEQ ID NO: 110) | | | | | |
| TAGLEEYSAM | HLA-B*35:01 | Q61L | 10 | 1.7686 | WB |
| (SEQ ID NO: 111) | | | | | |
| DTAGHEEY | HLA-A*25:01 | Q61H | 8 | 1.7688 | WB |
| (SEQ ID NO: 112) | | | | | |
| REEYSAMRDQY | HLA-A*30:02 | Q61R | 11 | 1.7827 | WB |
| (SEQ ID NO: 113) | | | | | |
| DILDTAGLEEY | HLA-A*25:01 | Q61L | 11 | 1.7871 | WB |
| (SEQ ID NO: 114) | | | | | |
| DILDTAGKEEYSAM | HLA-A*26:01 | Q61K | 14 | 1.7883 | WB |
| (SEQ ID NO: 115) | | | | | |
| LLDILDTAGREEYS | HLA-A*01:01 | Q61R | 14 | 1.7989 | WB |
| (SEQ ID NO: 116) | | | | | |
| AGKEEYSAMR | HLA-A*31:01 | Q61K | 10 | 1.8091 | WB |
| (SEQ ID NO: 117) | | | | | |
| DTAGKEEYSAMR | HLA-A*33:03 | Q61K | 12 | 1.8141 | WB |
| (SEQ ID NO: 118) | | | | | |
| AGHEEYSAM | HLA-C*01:02 | Q61H | 9 | 1.826 | WB |
| (SEQ ID NO: 119) | | | | | |
| LLDILDTAGKEEYS | HLA-A*01:01 | Q61K | 14 | 1.8335 | WB |
| (SEQ ID NO: 120) | | | | | |
| DILDTAGREEYSAM | HLA-A*26:01 | Q61R | 14 | 1.8399 | WB |
| (SEQ ID NO: 121) | | | | | |
| DTAGHEEYSAMR | HLA-A*33:03 | Q61H | 12 | 1.8422 | WB |
| (SEQ ID NO: 122) | | | | | |
| AGREEYSAM | HLA-C*14:02 | Q61R | 9 | 1.8632 | WB |
| (SEQ ID NO: 123) | | | | | |
| GREEYSAMRDQY | HLA-B*44:03 | Q61R | 12 | 1.8976 | WB |
| (SEQ ID NO: 124) | | | | | |
| HEEYSAMRDQYMR | HLA-A*68:01 | Q61H | 13 | 1.899 | WB |
| (SEQ ID NO: 125) | | | | | |
| GKEEYSAMRDQY | HLA-B*44:03 | Q61K | 12 | 1.901 | WB |
| (SEQ ID NO: 126) | | | | | |
| GHEEYSAMRDQY | HLA-B*44:03 | Q61H | 12 | 1.905 | WB |
| (SEQ ID NO: 127) | | | | | |
| GLEEYSAMRDQY | HLA-B*44:03 | Q61L | 12 | 1.9295 | WB |
| (SEQ ID NO: 128) | | | | | |
| DILDTAGLEEYSAM | HLA-A*26:01 | Q61L | 14 | 1.9383 | WB |
| (SEQ ID NO: 129) | | | | | |
| REEYSAMRDQYMR | HLA-A*31:01 | Q61R | 13 | 1.948 | WB |
| (SEQ ID NO: 130) | | | | | |
| ILDTAGKEEY | HLA-A*30:02 | Q61K | 10 | 1.9588 | WB |
| (SEQ ID NO: 131) | | | | | |
| AGREEYSAM | HLA-B*07:02 | Q61R | 9 | 1.9832 | WB |
| (SEQ ID NO: 132) | | | | | |

Table 1C. List of RAS.Q61-derived neo-peptides that are predicted to bind common HLA-alleles according to NetMHCpan 4.0
(*) amino-acid substitutions that were taken into account are: Arginine (R, Lysine (K), Leucine (L), Histidine (H).
(*) peptides of length 9-14AA were take into account
(*) default NetMHCpan parameters were used to define binders, i.e. strong binder (SB) if % Rank <= 0.5; weak binder (WB) if % Rank <= 2.

TABLE 1D

| | | | | | Binding |
|---|---|---|---|---|---|
| | binding HLA | RAS | peptide | % | status |
| peptide sequence | allele | variant | length | Rank | (SB/WB) |
| DTAGQEEYSAM (SEQ | HLA-A*26:01 | WT | 11 | 0.2735 | SB |
| ID NO: 133) | | | | | |
| DTAGQEEYSAM | HLA-A*25:01 | WT | 11 | 0.4443 | SB |
| (SEQ ID NO: 134) | | | | | |
| ILDTAGQEEY (SEQ | HLA-A*01:01 | WT | 10 | 0.1099 | SB |
| ID NO: 2) | | | | | |
| LLDILDTAGQEEY | HLA-A*01:01 | WT | 13 | 0.143 | SB |
| (SEQ ID NO: 135) | | | | | |
| CLLDILDTAGQEEY | HLA-A*01:01 | WT | 14 | 0.7697 | WB |
| (SEQ ID NO: 136) | | | | | |
| DILDTAGQEEY | HLA-A*01:01 | WT | 11 | 1.0481 | WB |
| (SEQ ID NO: 137) | | | | | |
| DILDTAGQEEY | HLA-A*26:01 | WT | 11 | 0.8175 | WB |
| (SEQ ID NO: 138) | | | | | |
| DILDTAGQEEYSAM | HLA-A*26:01 | WT | 14 | 1.9655 | WB |
| (SEQ ID NO: 139) | | | | | |
| DTAGQEEY | HLA-A*26:01 | WT | 8 | 1.7666 | WB |
| (SEQ ID NO: 140) | | | | | |
| DTAGQEEYSAMR | HLA-A*68:01 | WT | 12 | 0.8619 | WB |
| (SEQ ID NO: 141) | | | | | |
| DTAGQEEYSAMR | HLA-A*33:03 | WT | 12 | 1.9662 | WB |
| (SEQ ID NO: 142) | | | | | |
| GQEEYSAMRDQY | HLA-B*44:03 | WT | 12 | 1.874 | WB |
| (SEQ ID NO: 143) | | | | | |
| ILDTAGQEEY | HLA-A*29:02 | WT | 10 | 1.5352 | WB |
| (SEQ ID NO: 144) | | | | | |
| ILDTAGQEEY | HLA-A*30:02 | WT | 10 | 1.5942 | WB |
| (SEQ ID NO: 145) | | | | | |
| ILDTAGQEEYSAM | HLA-C*05:01 | WT | 13 | 0.9699 | WB |
| (SEQ ID NO: 146) | | | | | |
| ILDTAGQEEYSAM | HLA-C*08:02 | WT | 13 | 1.2923 | WB |
| (SEQ ID NO: 147) | | | | | |
| LDILDTAGQEEY | HLA-A*01:01 | WT | 12 | 1.8164 | WB |
| (SEQ ID NO: 148) | | | | | |
| LLDILDTAGQEEYS | HLA-A*01:01 | WT | 14 | 1.6584 | WB |
| (SEQ ID NO: 149) | | | | | |
| QEEYSAMRDQY | HLA-B*44:02 | WT | 11 | 0.8176 | WB |
| (SEQ ID NO: 150) | | | | | |
| QEEYSAMRDQY | HLA-B*18:01 | WT | 11 | 1.1461 | WB |
| (SEQ ID NO: 151) | | | | | |
| QEEYSAMRDQY | HLA-B*44:03 | WT | 11 | 0.6195 | WB |
| (SEQ ID NO: 152) | | | | | |

Table 1D: List of WT RAS.Q61-derived peptides that are predicted to bind common HLA-alleles according to NetMHCpan 4.0
(*) amino-acid substitutions that were taken into account are: Arginine (R, Lysine (K), Leucine (L), Histidine (H).
(*) peptides of length 9-14AA were take into account
(*) default NetMHCpan parameters were used to define binders, i.e. strong binder (SB) if % Rank <= 0.5; weak binder (WB) if % Rank <= 2.

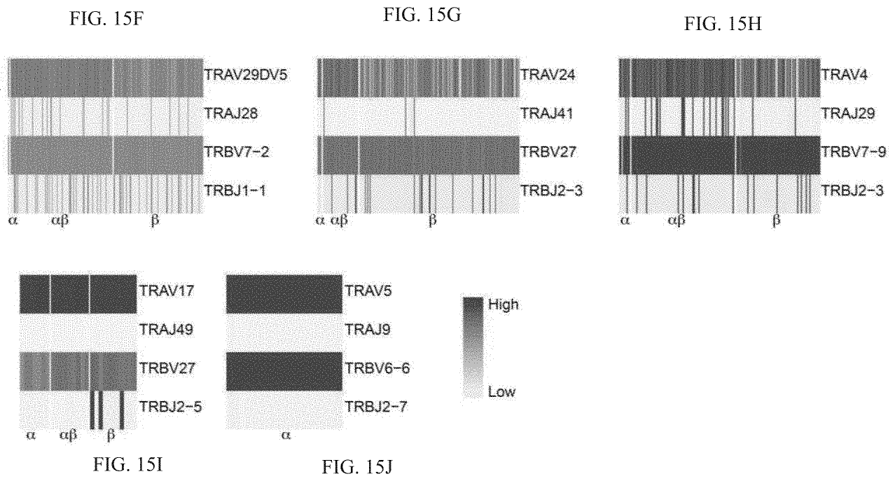

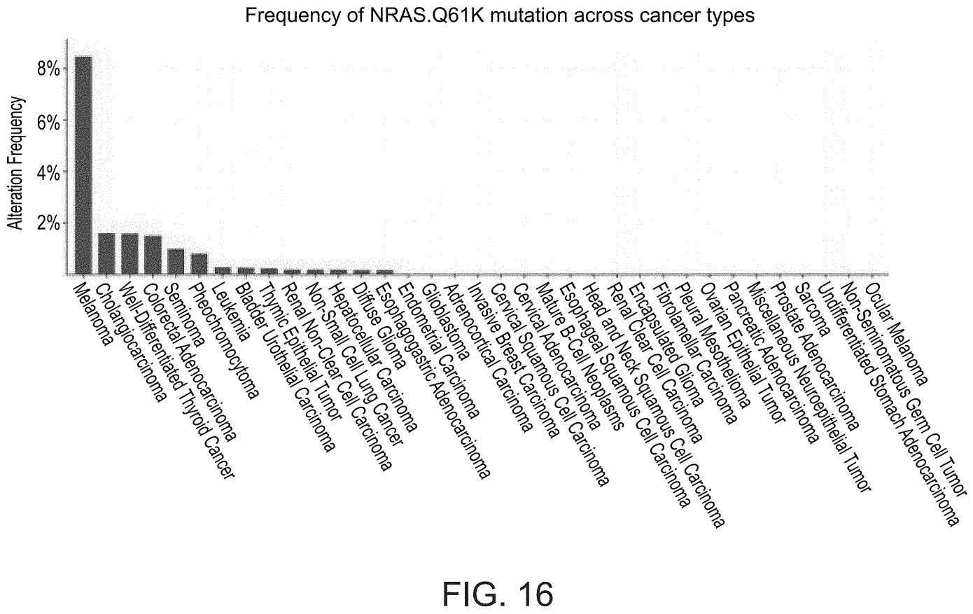

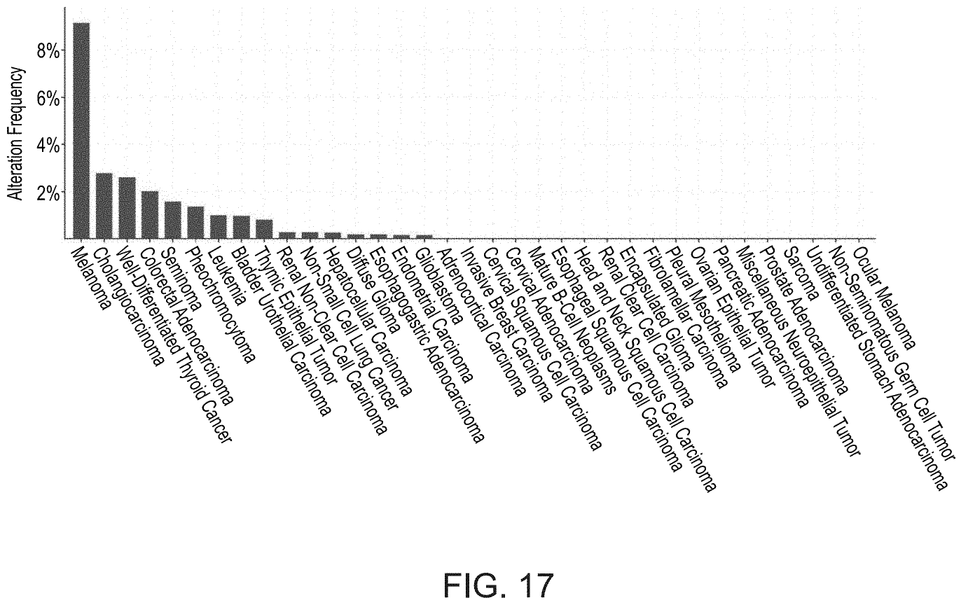

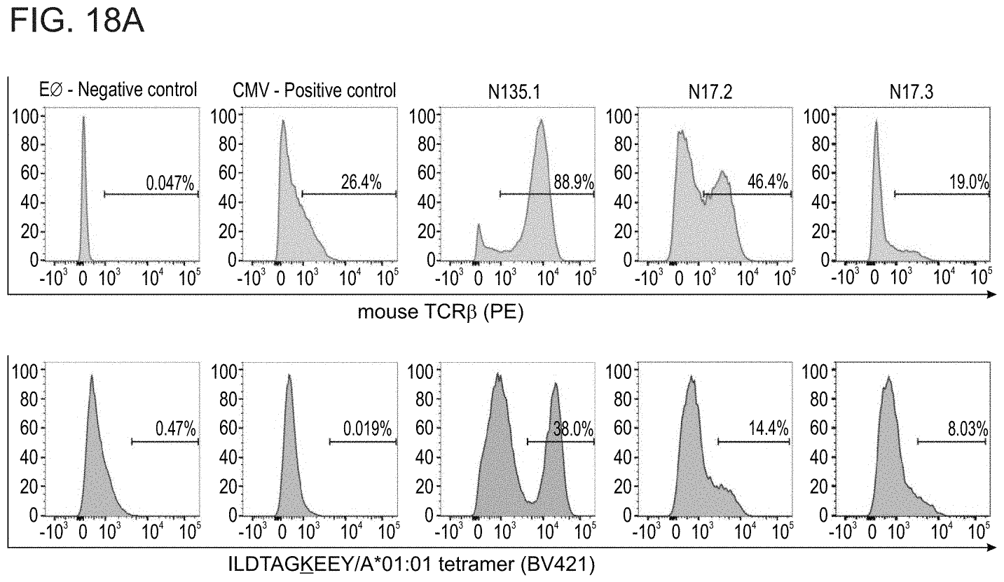

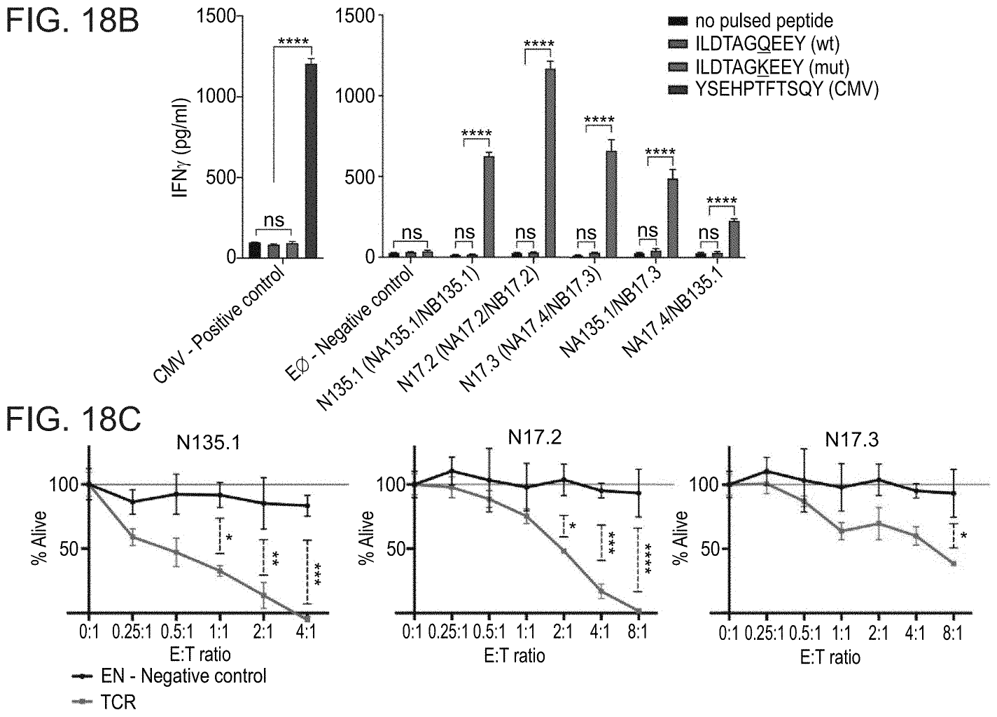

A careful assessment of this list of peptides revealed that, most often, the same peptide was predicted to bind in several amino-acid substitution variants (including also the wild-type variant, glutamine), leading to a count of 25 canonical peptides, with four predicted strong binders, and 21 predicted weak binders. In total, 26 HLA class-I alleles were predicted to bind RAS.61-derived peptides (FIGS. 2A and 5C). Interestingly, the highly frequent HLA allele A*01:01 was found to have compelling binding predictions (strong binding and found in about 8% of the samples with RAS.61 mutation, see below), and was thus selected for further analysis.

HLA-A*01:01 is known to be one of the most abundant class-I alleles in the general population. Consistently, our analysis of TCGA cancer patients revealed that 25.3% and 29.2% of pan-cancer and melanoma patients, respectively, possess at least one copy of this allele. As mentioned above, RAS.61 mutations are frequent cancerous events, with 3% of all cancer cases and 25.4% of melanoma cases affected. Most importantly, the frequency of HLA-A*01:01 does not diminish when restricted to the RAS.61-mutant population: 25.8% of RAS.61-mutant pan-cancer patients and 28% of RAS.61-mutant melanoma patients possess it. The above holds true also when analyzing large subgroups across various RAS isoforms and amino-acid substitutions (FIGS. 2B and 5D-E). As expected, the most frequent amino-acid substitutions at the RAS.61 position are arginine (R, 51%), lysine (K, 28.2%), leucine (L, 11.7%) and histidine (H, 10.1%). NRAS is the most abundantly mutated RAS isoform at position 61 (64.3% of RAS.61 mutations, 65.5% of patients with a RAS.61 mutation and HLA-A*01:01). NRAS.61 mutations appear in 24.3% of TCGA melanoma patients, with NRAS.Q61R noted in 11.7% of melanoma patients and NRAS.Q61K found in 8.7% of melanoma patients. All in all, 0.8% (6.6:1000) of pan-cancer and 7.1% of melanoma patients manifest the HLA-A*01:01/RAS.61 combination (FIGS. 2B and 5D-E), with 3% and 2.2% of TCGA melanoma patients possessing the HLA-A*01:01/NRAS.Q61R and HLA-A*01:01/NRAS. Q61K combination, respectively.

HLA-A*01:01 was predicted to bind the highest number of peptides, 21.7% of all predicted peptides, and 61.5% of the predicted strong binders. Peptides derived from all four amino-acid substitutions were predicted to strongly bind to HLA-A*01:01 (Table 1D).

It may be concluded that HLA-A*01:01/RAS.61 is a high-potential candidate combination for being a producer of recurrent neo-antigens. HLA-A*01:01/NRAS.Q61K is prevalent among cancer patients, especially those with melanoma. Should it be validated to be a neo-antigen-producing combination, thousands of individuals could benefit from its targeting yearly in the United States alone.

Direct Identification of a NRAS.Q61K-Derived Neo-Antigen in Tumor Cell Lines

Next, the present inventors set out to unbiasedly query the neo-antigen landscape of the NRAS.Q61K mutation in the context of HLA-A*01:01. To this end, they performed HLA-peptidomics on the 17T melanoma cell line, which possess the desired mutation/HLA combination. A previous whole-exome effort had determined the repertoire of somatic mutations for 17T³², based on the matched normal and metastatic tumor tissue from a suitable patient. They immunoaffinity purified pHLA complexes from 17T cell lysate. The peptide fraction was then eluted, followed by capillary chromatography and tandem mass spectrometric analysis of the HLA-bound peptides. Mass spectrometry results were analyzed using the MaxQuant software tool⁶⁰ and queried against the human proteome dataset (Uniprot), to which the amino acid changes corresponding to the mutations identified by the whole-exome sequencing were manually added, including the NRAS.Q61K variant. They detected 2356 peptides by mass spectrometry, including one NRAS.Q61K-derived neo-peptide—the nonamer ILDTAG [symbol] EEY (SEQ ID NO: 1) (FIG. 2D). No other neopeptides were detected.

Peptide identification accuracy was validated by comparing the endogenous peptide spectra to synthetic peptide spectra (FIG. 6). Both wild-type and mutant NRAS transcripts were expressed in the 17T tumor cell line (FIG. 7). To test the robustness of the presentation, a set of 3 additional tumor cell lines was compiled, all harboring the HLA-A*01:01 allele and NRAS.Q61K mutation (FIG. 2E). All cell lines were validated to express mutant NRAS transcripts (FIG. 7). The pHLA immunopurification from cell lysates and peptide elution was performed as described above on the three additional cell lines. High sensitivity absolute targeted mass spectrometry was employed to detect the neo-antigen across the available cell lines. Predetermined amounts of synthetic stable isotopically labeled ILDTAGKEEY (SEQ ID NO: 1) peptide (i.e., “heavy-peptide”) were spiked into the samples, enabling quantification of the identified endogenous peptide. Indeed, the endogenous neo-peptide was found in all three cell lines, in amounts ranging from 25 to 55 amol per sample (FIG. 2E).

It can be concluded that ILDTAG [symbol] EEY (SEQ ID NO: 1) is a robust, naturally processed, NRAS.Q61K-derived neo-peptide that is presented in the context of HLA allele A*01:01.

Mutant lysine of HLA-A*01:01/ [symbol] is free to interact with T-Cell Receptors

To gauge the nature of wild-type vs. neo-peptide binding to HLA, and to estimate the availability of the mutant residue, when in complex, to interact with T-cell receptors, the side-chains of decapeptides of interest, namely, ILDTAGKEEY (SEQ ID NO: 1) and ILDTAGQEEY (SEQ ID NO: 2), were threaded onto the backbone alignment of a previously resolved crystal structure depicting HLA-A*01:01 bound to an ALK decapeptide (PDB: 6at9)³⁴. The HLA structure was truncated to the peptide-binding domain, and the resulting peptide-HLA structures served as starting conformations for molecular dynamics (MD) simulations. After initial steric-clash minimization and equilibration, five independent simulation repeats were performed for each of the two complexes, each conducted for 500 ns. Both the mutant and wild-type peptides remained in close proximity to the HLA receptor throughout the duration of simulation. To focus on bound conformations, only output conformations in which the peptide's N- and C-termini were within 7 Å of the HLA anchor pockets B and F, respectively, were retained for further analysis. Docking served to produce additional binding conformation predictions, using the free-access web server interfaces FlexPepDock³⁵, ClusPro³⁶ and DINC³⁷. The simulation frames were clustered by HLA-peptide hydrogen-bonding interactions.

Analysis of hydrogen-bonding interactions present in predicted HLA-ras peptide complexes indicated that the mutant and wild-type peptides interact with HLA-A*01:01 in a highly similar manner. Previous work demonstrated that the combination of aspartic acid at position 3 (P3) and tyrosine at the C-terminus (Pa) form a canonical anchoring motif for HLA-A*01:01-bound decapeptides⁶⁵. In agreement with this, hydrogen bond are observed between the HLA receptor and these residues for both the wild-type and mutant variants (FIG. 8A). In contrast, P7 (residue 61) is predicted to form fewer hydrogen bonds with the HLA receptor. Consistently, while P7's backbone atoms remain within the peptide-binding groove throughout the simulation, its sidechain is highly solvent-exposed in both peptides. Bound simulated conformations thus indicate that the sidechain of P7 is directed toward the TCR-facing surface in a range of peptide conformations within the binding groove (FIG. 2C). The simulations also cautiously suggest that the mutant lysine in P7 occupies a narrower range of conformations, nearer to residues 147-156, compared to the wild-type glutamine (FIG. 8B). Further sampling of the conformational motions accessible to the HLA-peptide complexes is required to more precisely define the accessible conformations of P7. In conclusion, structural modeling indicates that residue 61 faces out of the HLA pocket and is freely available to interact with T-cell receptors. This, in turn, suggests that differential mutant vs. wild-type TCR binding may be possible.

Immunogenicity Evaluation of the HLA-A*01:01/ [symbol] Hotspot Neo-Antigen

To evaluate the immunogenicity of the NRAS.Q61K neo-antigen, two melanoma cell lines that harbor the HLA-A*01:01/NRAS.Q61K combination, 17T and 135T were analyzed. Synthetic mutant (ILDTAG [symbol] EEY-SEQ ID NO: 1) or wild-type (ILDTAG [symbol] EEY-SEQ ID NO: 2) peptide was pulsed on either one of three commercial, A*01:01 expressing, Epstein bar virus (EBV)-transformed B cells³⁵. No other HLA class-I allele was shared among all three B-cell lines, and no other HLA class-I allele expressed by these cell lines was predicted to bind the neo-peptide at hand (Table 2).

TABLE 2

| IHW | | | | | | | Ethnic |
|---|---|---|---|---|---|---|---|
| Number | Locus A* | | Locus B* | | Locus Cw* | | Group |
| IHW01161 | A*01:01 | A*68:01:02 | B*08:01 | B*13:02 | B*07:01 | B*03:03 | Caucasian |
| IHW01113 | A*01:01 | A*02:01 | B*57:01 | B*44:02:01:01 | B*06:02 | B*05:01 | Caucasian |
| IHW01070 | A*01:01 | A*02:01 | B*08:01 | B*40:01 | B*07:01 | B*03:04:01 | Caucasian |

The peptide-pulsed B-cells were co-incubated with either 17TIL or 135TIL overnight, followed by measurement of peptide stimulated interferon-γ (IFN-γ) release from the TIL by means of enzyme-linked immunosorbent assay (ELISA). As depicted in FIG. 3A, the mutated NRAS.Q61K peptide stimulated a clear interferon-γ (IFN-γ) signal, whereas the wild-type version did not elicit significant increase over a non-pulsed B-cell control. Peptide titration assays further confirmed specific TIL reactivity towards the HLA-A*01:01/NRAS.Q61K neo-antigen, for both 17TIL and 135TIL (FIG. 3B). While TIL response was non-significant and did not depend on peptide concentration for the wild-type variant, a clear dose-response relationship manifested for the neo-peptide in both TIL populations, with 17TIL and 135TIL showing significant response at minimum concentrations of 10 ng/ml and 1 ng/ml respectively.

The HLA-A*01:01/ILDTAG [symbol] EEY-SEQ ID NO: 1—neo-antigen is thus shown to be recognized by TIL from two unrelated patients with tumors bearing the HLA-A*01:01/NRAS.Q61K combination.

To further explore the neo-antigen reactive TIL sub-population, the bulk TIL was stained with a fluorophore conjugated tetramer. Flow cytometry analysis of stained TIL revealed that 22.6% of bulk 17TIL and 75.1% of bulk 135TIL were neo-antigen specific (FIG. 3C). Fluorescence-activated cell sorting was used to tetramer-sort the bulk TIL populations. As a testament for tetramer sensitivity, tetramer positive sub-populations of 17TIL and 135TIL retained their IFNγ release capabilities, in response to neo-peptide pulsed B-cells, while the tetramer negative sub-populations did not show significant reactivity towards the neo-antigen (FIG. 9). An in-vitro killing assay was employed to test the cytotoxic capacity of tetramer positive TIL populations. GFP-labeled 17T or 135T melanoma, were co-incubated with their cognate tetramer-positive, tetramer-negative or bulk TIL populations at varying effector to target ratios. As can be seen in FIG. 3D, tetramer positive TIL were able to eliminate the melanoma, and showed the expected dose-response aptitude. Tetramer-positive 17TIL showed significant advantage in killing capacity over both tetramer-negative and bulk 17TIL. It can be concluded that the HLA-A*01:01/ILDTAG [symbol] EEY (SEQ ID NO: 1) hotspot neo-antigen is immunogenic, with T-cells targeting it being able to eliminate the antigen-expressing melanoma.

Identification of HLA-A*01:01/ [symbol] Specific TCRs and Characterization of Antigen-Targeting TIL

As the HLA-A*01:01/NRAS.Q61K combination is expected to appear in 2.2% of melanoma cases, and apply to 1.4:1000 individuals pan-cancer, it is important to identify TCRs that target the HLA-A*01:01/ILDTAG [symbol] EEY (SEQ ID NO: 1) neo-antigen for future research and clinical applications. To characterize the neo-antigen-specific TCR-repertoire within the bulk TIL, RNA based sequencing of expressed TCRα and TCRβ chains on tetramer sorted and bulk CD4-TIL. For both 17TIL and 135TIL, and in all three subpopulations analyzed, i.e. tetramer-positive, tetramer-negative and bulk CD4-, hundreds to thousands of distinct TCR chains, defined by productive amino acid sequences of the CDR3 region, were detected. However, restricting to those that consist 1% and above of the transcripts, clear oligoclonal distributions emerged, with 11 distinct TCRβ (12 TCRα) amino-acid sequences dominating CD4-17TIL, and 6 TCRβ (7 TCRα) chains dominating CD4-135TIL (accounting for 75.4% and 92.2% of the repertoires, respectively, see FIGS. 11A-B and 12A-B). Similarly-shaped oligoclonal distributions were observed for the tetramer-positive and tetramer-negative sorted subpopulations (FIGS. 11A-G and 12A-G). To increase signal reliability, sequences that were considerably enriched in frequency in the tetramer-positive subpopulation as compared to the tetramer-negative subpopulation were selected for further analysis. As depicted in FIG. 4A-B, this pin-pointed four TCRβ and five TCRα chains with both transcript frequency of at least 1% and frequency-enrichment of at least 100-fold when comparing the 17TIL tetramer-positive sub-population with the tetramer-negative one, suggesting that multiple neo-antigen-specific clones exist within 17TIL. The cumulative frequencies for these four TCRβ chains were 68.5%, 3% and 0.005% in the tetramer-positive, bulk CD4- and tetramer-negative populations, respectively. The most abundant TCRα chain in the tetramer-positive sub-population, TRBV27/CASSLVSTPLPKETQYF (SEQ ID NO: 200)/TRBJ2-5 (denoted NB17.1), consisted of 50.9% of the transcripts in this group (see FIG. 41). Similarly, for TCRα, cumulative frequencies for the five chains of interest were 68.9%, 3.2% and 0% in the tetramer-positive, bulk CD4- and tetramer-negative populations, respectively. The most abundant TCRα chain in the tetramer-positive sub-population, TRAV17/CATDCKNQF (SEQ ID NO: 199)/TRAJ49 (denoted NA17.1), consisted of 44.1% of the transcripts in this group. Interestingly, the α and β frequency distributions were consistent enough to enable an educated guess regarding chain pairing (FIG. 11G).

The full list of identified TCR receptors is presented in Table 3, herein below:

TABLE 3

| | | CDR | | CDR3 | V region | J region |
|---|---|---|---|---|---|---|
| Chain | | amino acid | | nucleic acid | nucleotide | nucleotide |
| name | V region | sequence | J region | sequence | sequence | sequence |
| NA17.1 | TRAV17 | 199 | TRAJ49 | 211 | 223 | 236 |
| NB17.1 | TRBV27 | 200 | TRBJ2-5 | 212 | 224 | 237 |
| NA17.2 | TRAV5 | 201 | TRAJ9 | 213 | 225 | 238 |
| NB17.2 | TRBV6-6 | 202 | TRBJ2-7 | 214 | 226 | 239 |
| NA17.3 | TRAV16 | 203 | TRAJ52 | 215 | 227 | 240 |
| NB17.3 | TRBV6-1 | 204 | TRBJ1-1 | 216 | 228 | 241 |
| NA17.4 | TRAV19 | 205 | TRAJ17 | 217 | 229 | 242 |
| NB17.4 | TRBV12-3 | 206 | TRBJ1-1 | 218 | 230 | 243 |
| NA17.6 | TRAV19 | 207 | TRAJ39 | 219 | 231 | 244 |
| NB17.5 | TRBV6-6 | 208 | TRBJ1-1 | 220 | 232 | 245 |
| NA135.1 | TRAV19 | 209 | TRAJ39 | 221 | 234 | 246 |
| NB135.1 | TRBV6-1 | 210 | TRBJ1-1 | 222 | 235 | 247 |

For 135TIL, a single pair of TCRβ/TCRα chains met the above criteria (see FIG. 4C-D, I, chains NA135.1 and NB135.1), with frequencies of 89.6% and 85% within tetramer-positive 135TIL, respectively. Interestingly, chain NA135.1, identified for 135TIL, bears sequence similarity to NA17.4, the fourth most frequent TCRα candidate identified for 17TIL.

To add another layer to the analysis, the TCRs of reactive TIL were independently sequenced. Bulk 17TIL and 135TIL were each co-cultured in 1:1 ratio with the cognate melanoma cell line overnight. TIL were then stained and sorted according to their expression of activation marker 4-1BB. 16.1% of bulk 17TIL and 16.8% of bulk 135TIL expressed 4-1BB in response to cognate melanoma (background 4-1BB expression without stimulation was observed in 0.058% and 0% of the cells respectively, see FIG. 10E-H). All of the neo-antigen-specific TCR chains, as identified above, were found to take part in the 4-1BB+ repertoire. Since 4-1BB is preferentially expressed on activated CD8+ T-cells, and the majority of bulk 17TIL (˜70-80%) are CD4+ cells, the present inventors compared TCR chain frequencies between the 4-1BB+ and bulk CD4-subpopulations. Three chains: NA17.1 (p=0.04), NA17.2 (p=0.045) and NB17.1 (p=0.05) were found to be significantly enriched in the 4-1BB+ subpopulation (binomial one-sided test, with Benjamini Hochberg correction, FIG. 4E-H). These experiments show that several functional TCRs in 17TIL, and one TCR in 135TIL, are directed against HLA-A*01:01/ILDTAGKEEY (SEQ ID NO: 1).

Single-Cell Profiling of CD8+17TIL and the Neo-Antigen-Specific Subset

Through bulk TCR sequencing of tetramer-positive 17TIL, the present inventors identified those TCRα and TCRβ chains that mediate the recognition of HLA-A*01:01/ILDTAGKEEY (SEQ ID NO: 1). Next, the inventors sought to further characterize the transcriptional profile of neo-antigen-specific cells in response to cognate melanoma, and to compare it to other CD8+ populations within 17TIL. To this end, they performed single-cell RNA and TCR sequencing on 17TIL cells after overnight co-incubation with the 17T cell line (see Methods). After quality control and in-silico filtering for CD8+ cells (see Methods), a total of 2341 cells were retained.

Unsupervised clustering analysis uncovered six stable CD8+ subgroups in the data, each with a distinct expression profile (FIG. 13A, D). They used a predefined marker list to score the clusters on the scales of cytotoxicity, exhaustion and proliferation (FIG. 13F-H). Cluster 3 (382 cells) scored as the most highly cytotoxic cluster (Wilcoxon test with Benjamini Hochberg correction, see FIG. 13F). An abundant set of cytotoxicity-related markers were found to be differentially expressed in cluster 3, including: CCL3, GNLY, 4-1BB (TNFRSF9), IFNG, CCL4, CST7, GZMH, GZMB, NKG7 and PRF1. Clusters 1 (855 cells), 5 (259 cells) and 6 (35 cells) scored more cytotoxic than clusters 2 (530 cells) and 4 (280 cells). Both cluster 1 and cluster 3 scored high on the exhaustion scale, and were found significantly more exhausted than the other clusters (Wilcoxon test with Benjamini Hochberg correction, see FIG. 13G). Exhaustion markers differentially expressed in cluster 3 included: TIM-3 (HAVCR2), LAG3, SLA, TNFRSF1B, CTLA4 and CD39 (ENTPD1). Cluster 1 differentially expressed an extensive array of known markers of dysfunction, including TIGIT, PD1 (PDCD1), CTLA4, CD39, TIM-3, BATF, LAG3 and the transcription factor TOX. CD39, and its co-expression with CD103 (ITGAE, also highly differentially expressed in cluster 1) have been previously suggested to distinguish tumor-reactive cells within TIL populations. The coupling of exhaustion to cytotoxicity (as is apparent for clusters 3 and 1) is consistent with an activation-dependent exhaustion program, as was previously suggested. Other clusters, such as clusters 6 and 2, exhibit cytotoxicity without exhaustion.

Interestingly, layilin (LAYN), a recently identified marker for TIL exhaustion in hepato-cellular carcinoma as well as for poor prognosis in gastric and colorectal malignancies, came up highly differentially expressed in cluster 1. Leukocyte-associated immunoglobulin-like receptor 2 (LAIR2, CD306), a decoy soluble receptor for the immunoinhibitory membranal LAIR1, was found to be the most highly differentially expressed gene in cluster 3. T-cell specific expression of microRNA-155 (MIR155HG), also differentially expressed by cluster 3, have been recently deemed necessary for optimal anti-tumor immunity, and predicted a favorable outcome in human melanoma patients.

Cluster 5 emerged as the single most highly proliferative cluster based on the G2/M marker list (see FIG. 13H). Cell-cycle related genes that were differentially expressed in cluster 5 include: MKI67, TOP2A, STMN1, CENPF, MCM7 and TUBB4B.

TCR data was available for 1443 of the 2341 cells included in the analysis, with paired TCRα and TCRβ chains in 644 cells. In 674 cells only the TCRβ chain was sequenced productively, while a single TCRα chain was detected in 90 cells. Dual TCRα receptors were detected in seventeen cells, and dual TCRβ receptors appeared in eleven cells. Other supernumerary combinations were present in a total of seven cells. 60% of dual TCRα receptors, but only a single dual TCRβ combination, appeared in more than one cell, suggesting the higher frequency of cells expressing two different TCRα chains. Paired TCRα and TCRβ chains, and dual TCR combinations appearing in two or more cells were used to define T-cell clones (see methods).

In agreement with bulk TCR sequencing experiments, few expanded clones dominated the TCR repertoire obtained by single-cell analysis (see FIG. 14). For clonal analysis, three main groups were analyzed: (1) neo-antigen-specific clones; (2) the three most highly expanded clones; and (3) the bulk of non-expanded clones.

Initially, cells that express the two most frequent neo-antigen-specific TCR chains for 17TIL in bulk experiments NA17.1 and NB17.1 were identified. Their pairing was confirmed at the single-cell level by nine cells, thus defining clone N17.1 (i.e. NRAS specific clone 17.1). Together with 11 additional single-NB17.1 cells, and 7 additional single-NA17.1 cells, the N17.1 clone aggregated to a total of 27 cells (see methods and FIG. 15D). A single cell in the dataset confirmed the pairing of NA17.2 with NB17.2 (herein clone N17.2). This cell did not pass quality control filters (suspect as doublet due to high UMI count), however five single alpha chain cells pertaining to N17.2 were retained for gene-expression analysis (FIG. 13). Two cells matched NB17.3 with both NA17.3 and NA17.4 in a dual receptor (clone N17.3). These two cells failed to pass quality filters due to high mitochondrial gene expression and high UMI count, and were thus excluded from further analysis.

The three most highly expanded CD8+ clones for 17TIL, E17.1, E17.2 and E17.3, comprised of 465, 253 and 163 cells, respectively. Non-expanded clones, defined by TCRs that were detected no more than two times in our dataset, consisted of 100 different receptors and 112 cells in total.

Mapping clonal annotations onto the gene-expression cluster space, we found high within-clone transcriptional homogeneity (p=1.8×10⁻⁴⁶, X²; see FIG. 13B). Most of the cells of both neo-antigen-specific clones, N17.1 and N17.2, were found in the same functional cluster—the highly cytotoxic cluster 3 (93% and 80% of the cells, p=0.01 and p=0.02, respectively; standardized residuals analysis with Benjamini Hochberg correction). E17.3 was similarly enriched in cluster 3 (63% of the cells, adj. p<0.01), though at a different sub-cluster than the NRAS-specific clones. E17.1 was found to be enriched within cluster 1 (76% of the cells, adj. p<0.01). Clone E17.2 localized to both cluster 1 and cluster 4, with its cells grouped together across these clusters (48% and 44% of the cells, adj. p<0.01 and p=0.02 respectively, see FIG. 13A-C). Interestingly, the TCR-heterogenous non-expanded subpopulation tended to cluster together in cluster 2 (70% of the cells, adj. p<0.005), and were stably enriched in a single cluster under varying clustering parameters. Taken together with the low cytotoxicity and exhaustion scores observed for cluster 2, this may suggest a unifying transcriptional profile for bystander TIL.

As would be expected for a cell-cycle related profile, an assortment of cells, from all of the described clones, mapped onto cluster 5.

To gain further insight into the unique features of the NRAS neo-antigen-specific clones N17.1 and N17.2, we contrasted them with E17.3, which also maps onto cluster 3. As evident in FIG. 5E, LAIR2 tops the list of differentially expressed genes, marking it a unique marker of the neo-antigen-specific clone. Other highly differential genes for N17.1 and N17.2 include FABP5, a marker of tissue-resident memory T-cells, RBPJ—a transcription factor that was found to correlated with TIL dysfunction⁴⁹, KLRD1, KLRC3 and KLRC1. Interestingly, N17.1 was found to clonally express the TCRdelta gene TRDV1 and TCRγ gene TRGV8, in addition to its TCRγβ receptor.

Validation of Individual Neoantigen-Specific TCRα/TCRβ Pairs

The present inventors set out to validate the neoantigen-specificity and functional-competence of TCR candidates, that were deduced based on TCR sequencing data for 17TIL and 135TIL, i.e. N17.1={NA17.1, NB17.1}, N17.2={NA17.2, NB17.2}, N17.3={NA17.3/NA17.4, NB17.3} and N135.1={NA135.1, NB135.1}. To this end, they electroporated in-vitro transcribed mRNA transcripts, pertaining to the TCRα/TCRβ pairs of interest, into healthy donor peripheral blood T-lymphocytes (see methods section), and evaluated tetramer binding as well as neoantigen specific IFNγ secretion and killing capacity. At 6-30 hours post electroporation, cells were stained for flow cytometry analysis (FIG. 18A). As expected, cells electroporated with N17.2 and N135.1 stained positive with the ILDTAGKEEY (SEQ ID NO: 1)/A*01:01 tetramer, thus confirming their neoantigen binding capacity. N17.3 represents a dual TCRα clone in 17TIL single-cell data, i.e. cells of this clone express two different TCRα chains (NA17.3, NA17.4) and one TCRβ chain (NB17.3). They therefore tested both combinations with electroporation, confirming N17.3={NA17.4, NB17.3} to be the productive neoantigen-specific chain pairing (see FIG. 18A). The NA17.3/NB17.3 combination did not elicit tetramer binding, despite adequate surface expression (data not shown). They further tested the functional specificity of the different TCRs using IFNγ ELISA (see methods). The A*01:01+ B-LCL IHW01161 was pulsed with 10 μM of either the wild-type (ILDTAGQEEY (SEQ ID NO: 2) or mutant (ILDTAGKEEY (SEQ ID NO: 1) peptide, or incubated at the same conditions with an equal volume of plain DMSO (‘no-pulsed peptide’ control). The IHW01161 presenting cells were subsequently co-incubated at 1:1 ratio with electroporated T-lymphocytes. Supernatant IFNγ levels were measured via ELISA after overnight co-incubation. As depicted in FIG. 18B, N17.2, N17.3 and N135.1 all induced significant, neoantigen-specific, IFNγ release from the transfected cells. Interestingly, there is an amino-acid sequence similarity between receptors N135.1 and N17.3, both for the α and for the β chains. To test the functional correlate of this similarity, chain combinations were swapped, NA135.1/NB17.3 and NA17.3/NB135.1, using the TCR electroporation system. Strikingly, these hybrid TCRs, bringing together TCR chains from two unrelated patients, were functionally potent (see FIG. 18B). Swapping the chains of other TCRs, that are neoantigen-specific but unrelated in sequence, did not yield neoantigen specificity, as measured by tetramer staining and IFNγ release assays (data not shown). Taken together, the present findings suggest positive selection towards the identification of ILDTAGKEEY/A*01:01 neoantigen, with convergence of sequence within patient and, remarkably, also across patients. With neoantigen binding and specific reactivity established, the present inventors turned to test the most important functional capacity for TCRs—targeted killing of neoantigen-expressing tumor cells. To this end, they incubated electroporated donor lymphocytes with GFP-expressing 17T melanoma cell-line at different effector to target (E:T) ratios (see methods). As can be seen in FIG. 18C, N135.1, N17.2 and N17.3 are all able to kill 17T.

### REFERENCES

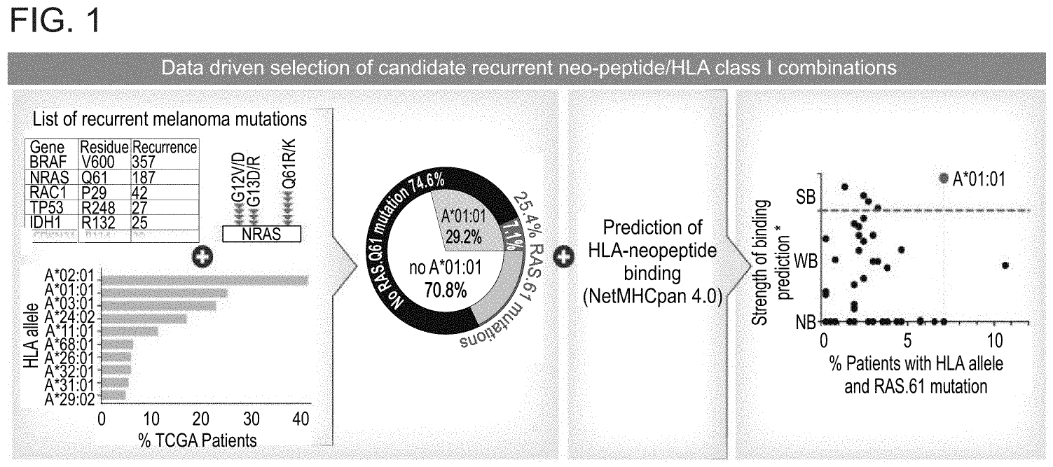

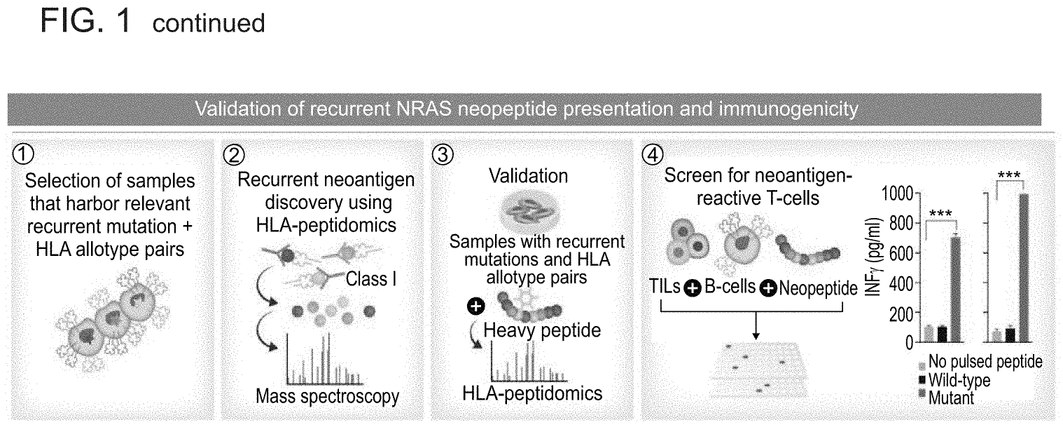

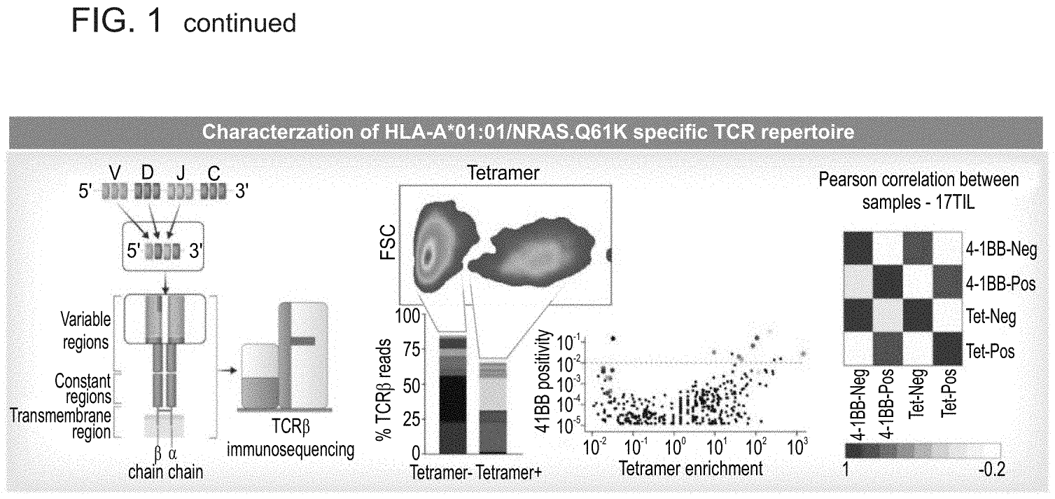

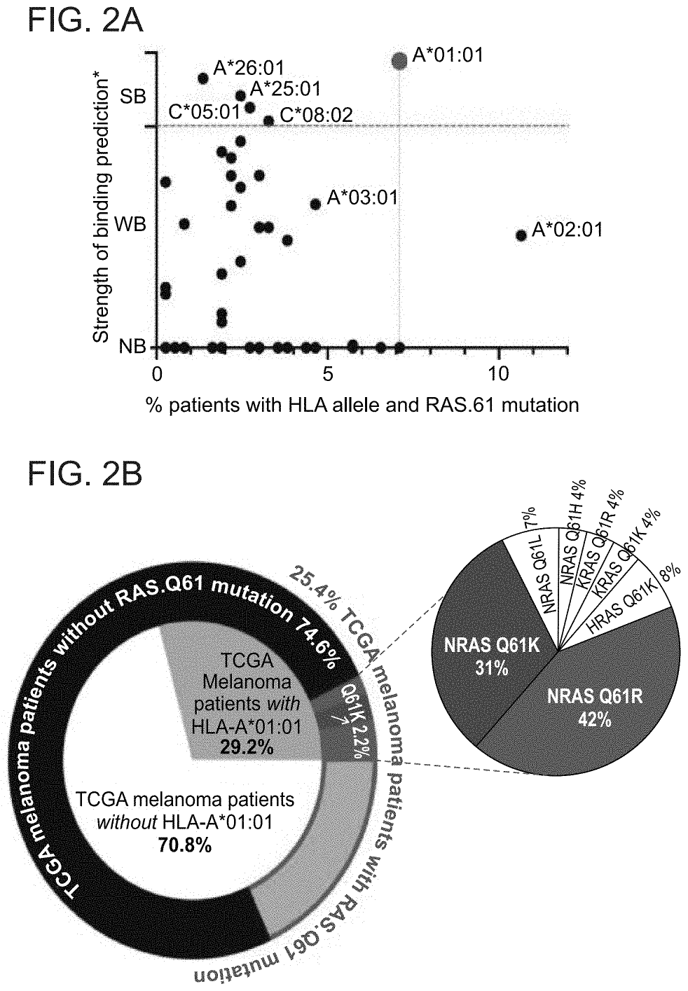

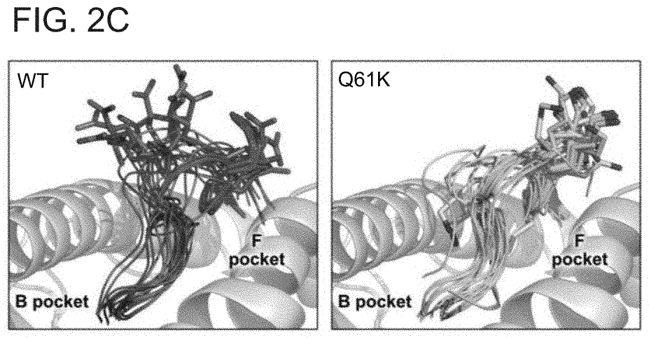

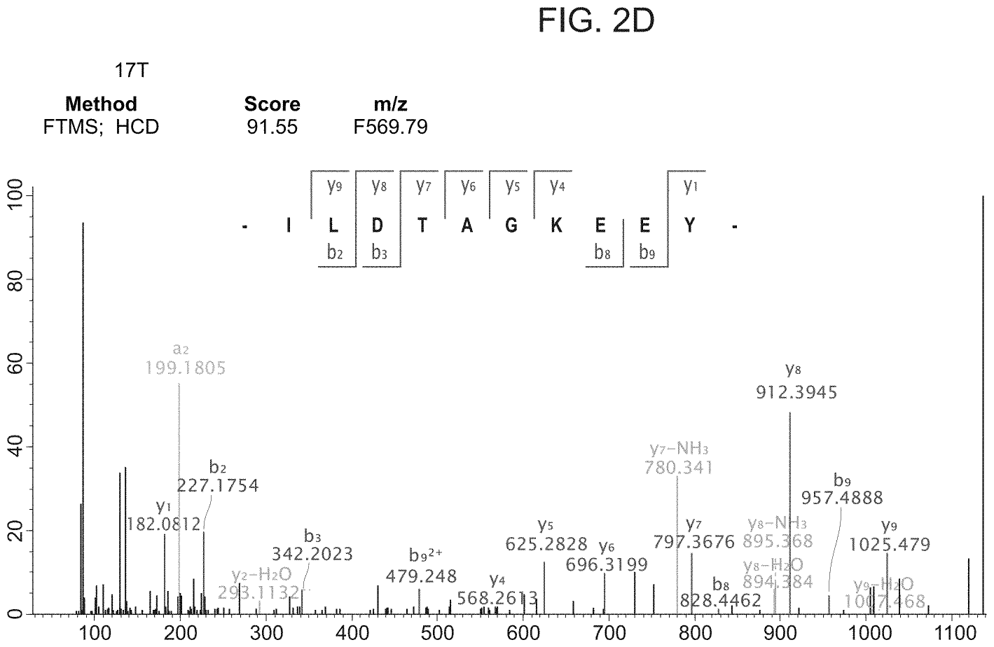

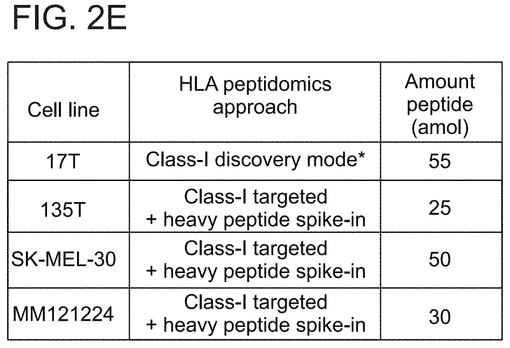

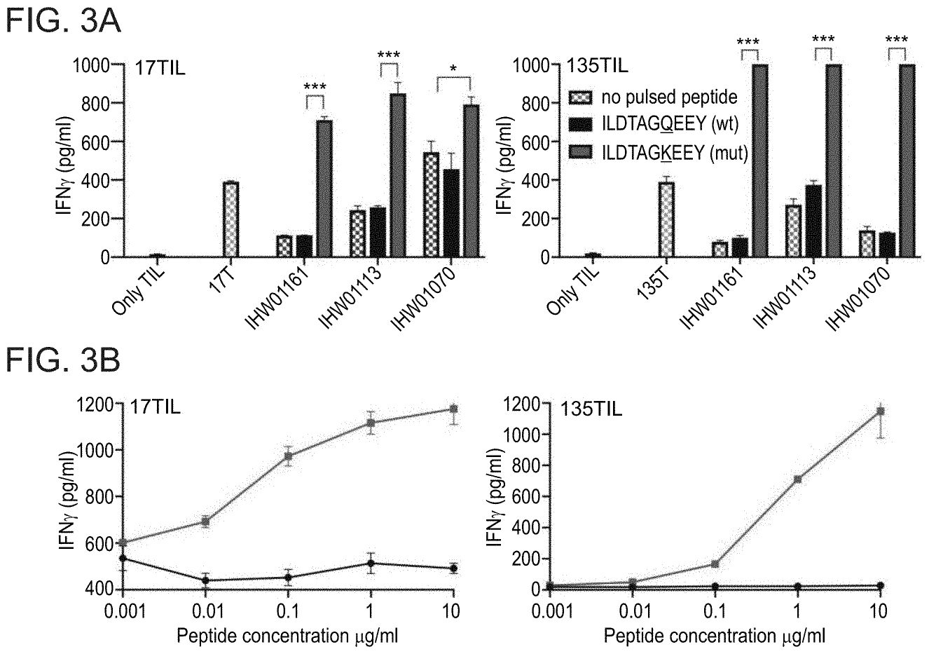

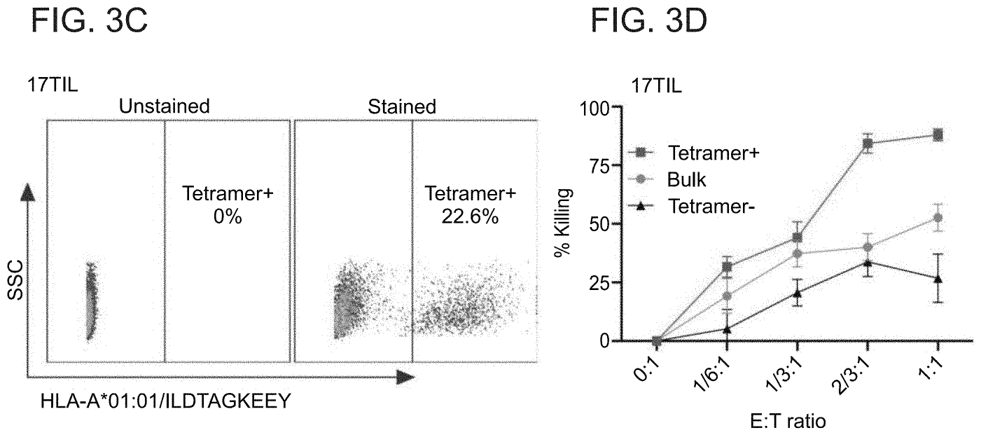

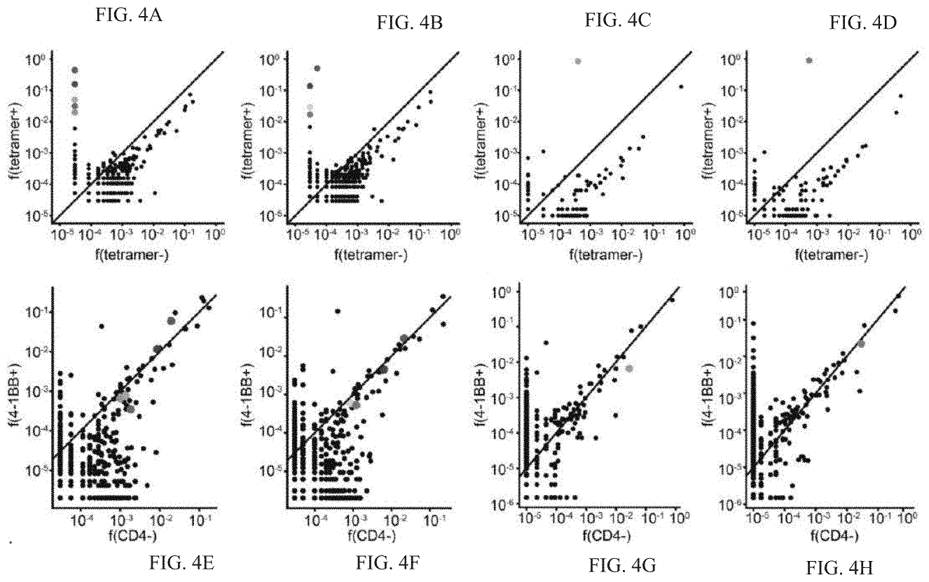

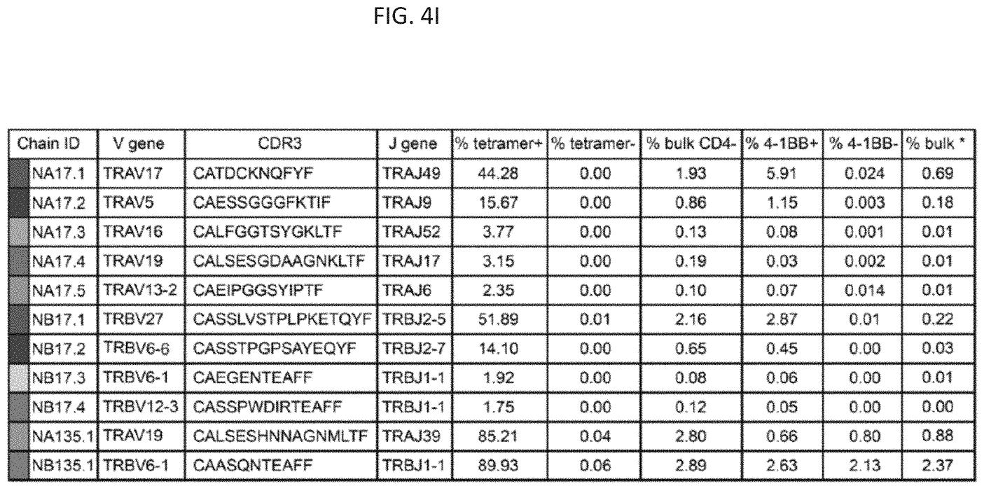

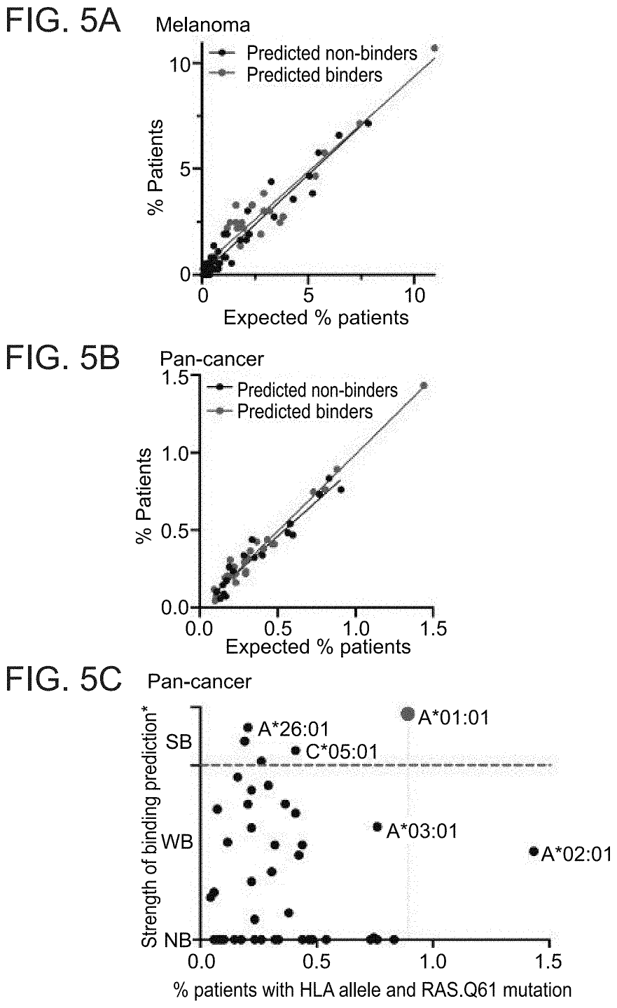

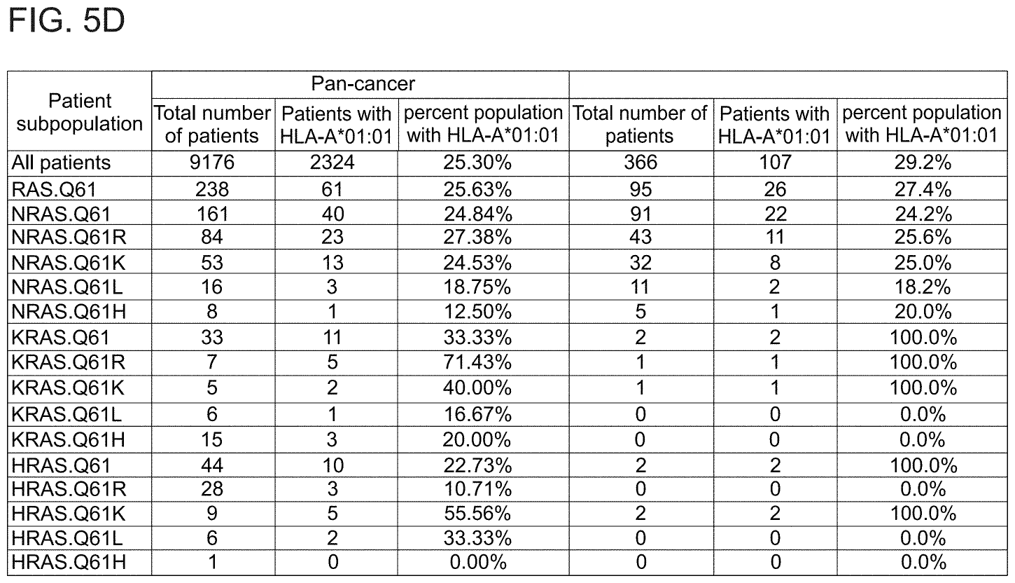

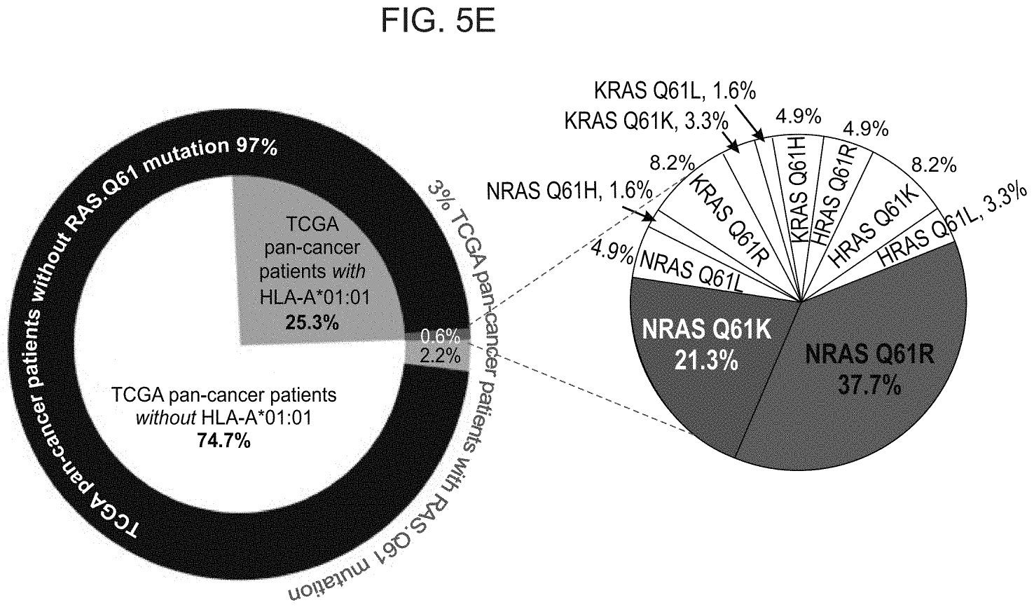

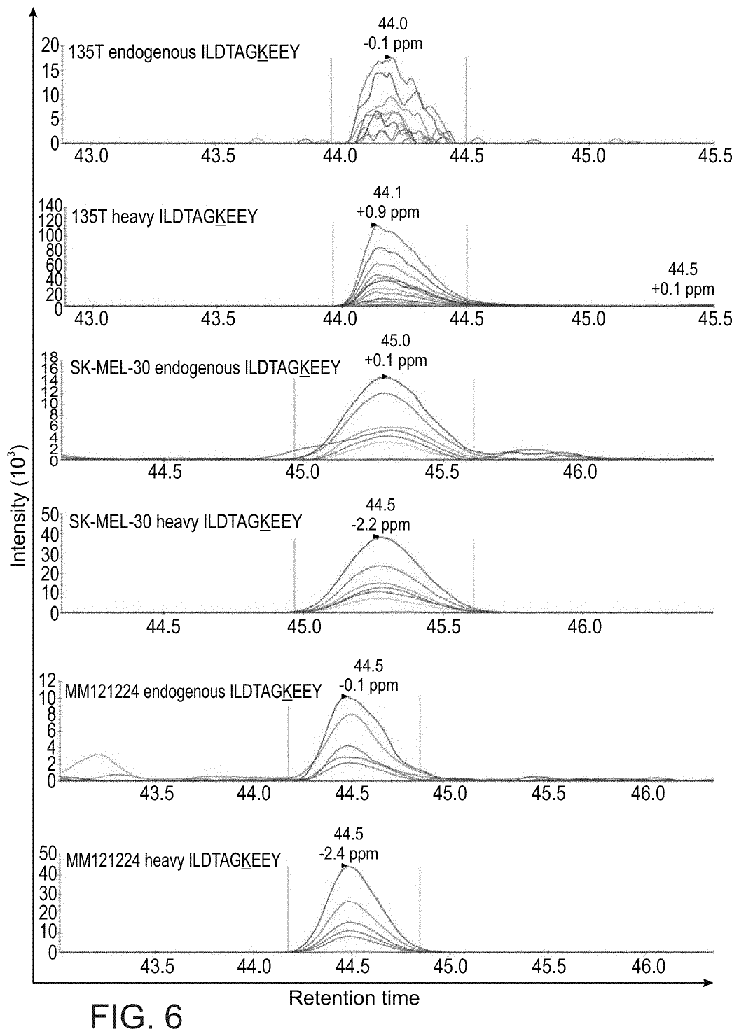

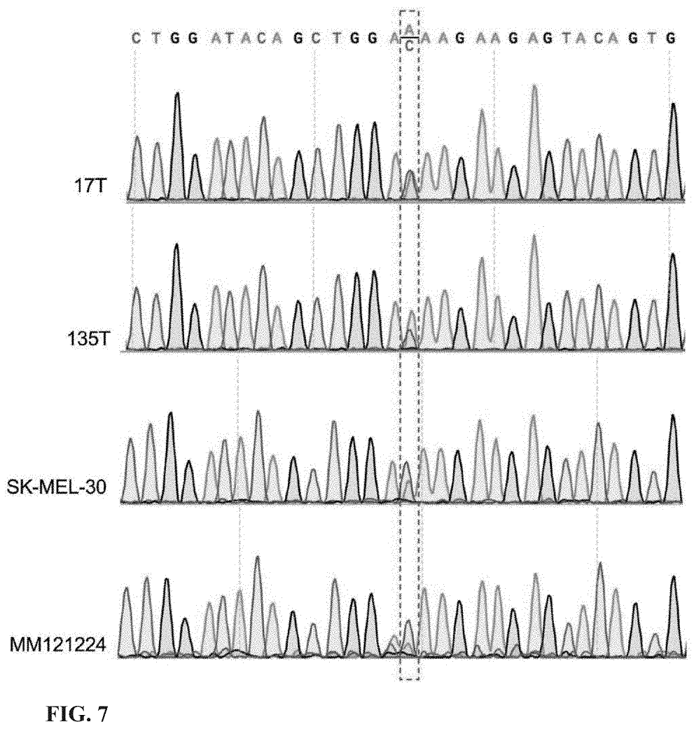

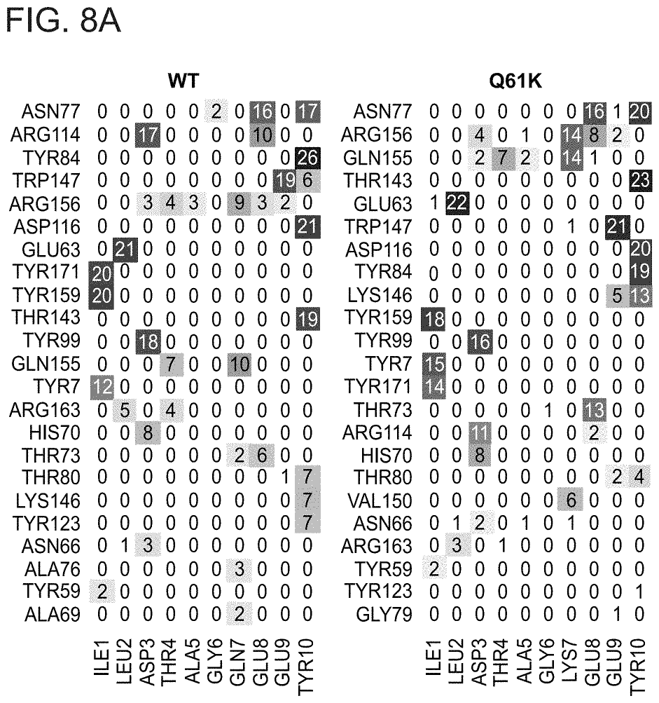

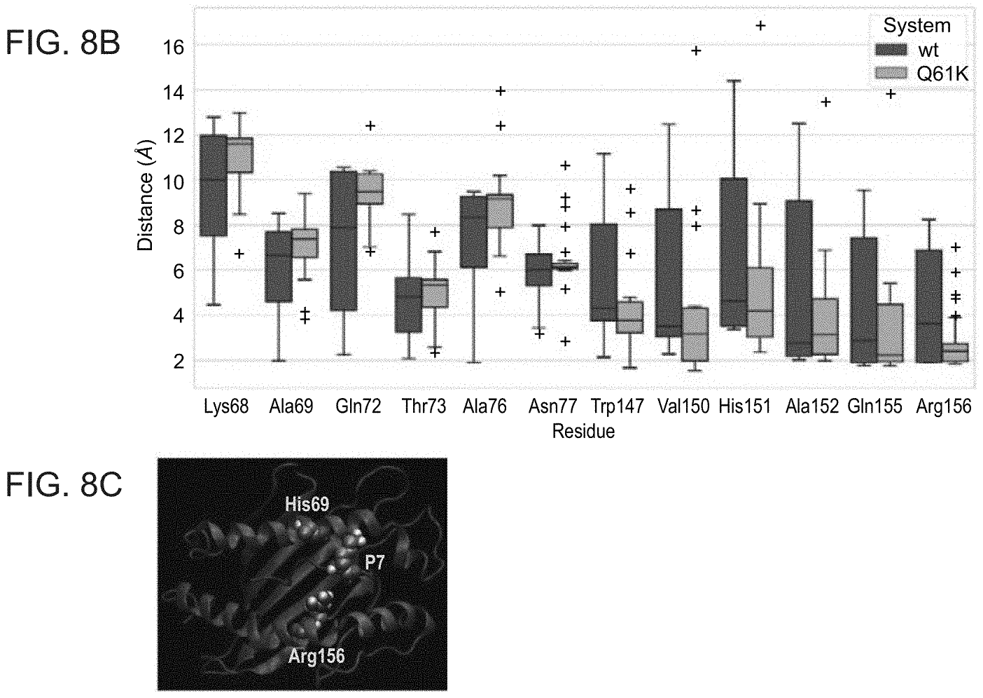

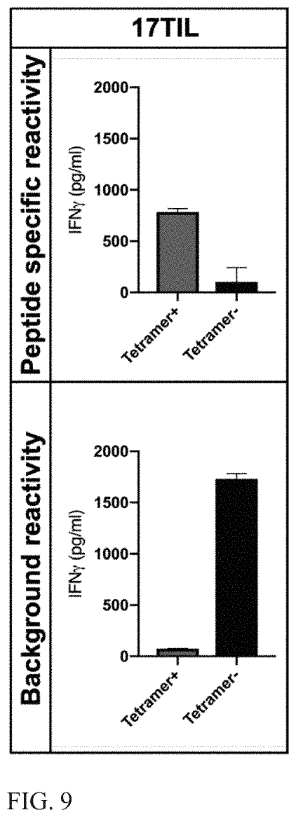

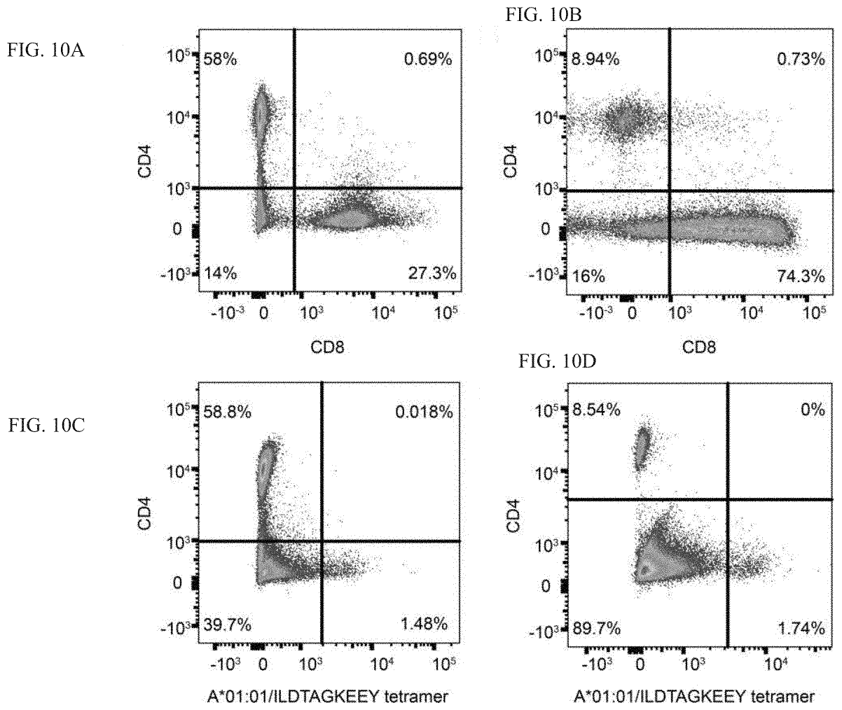

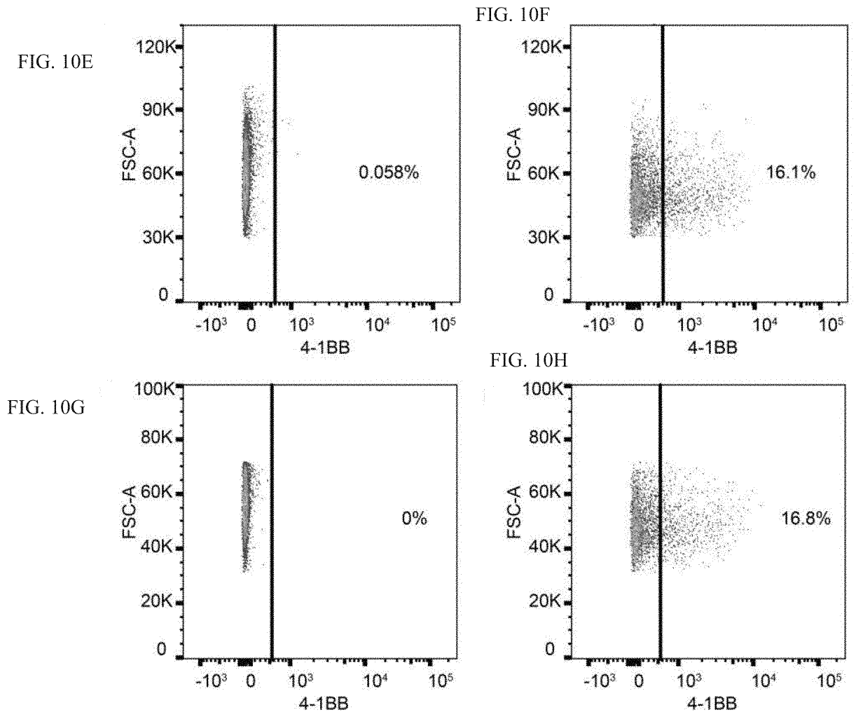

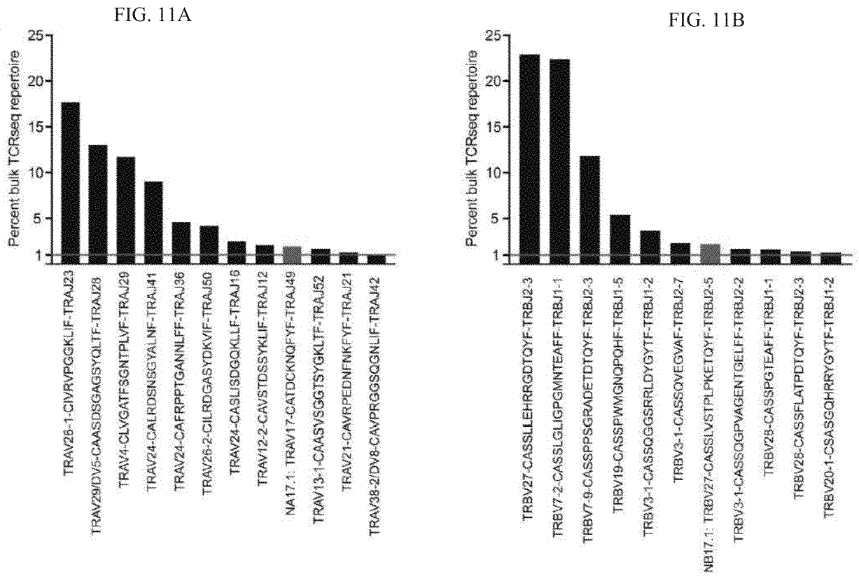

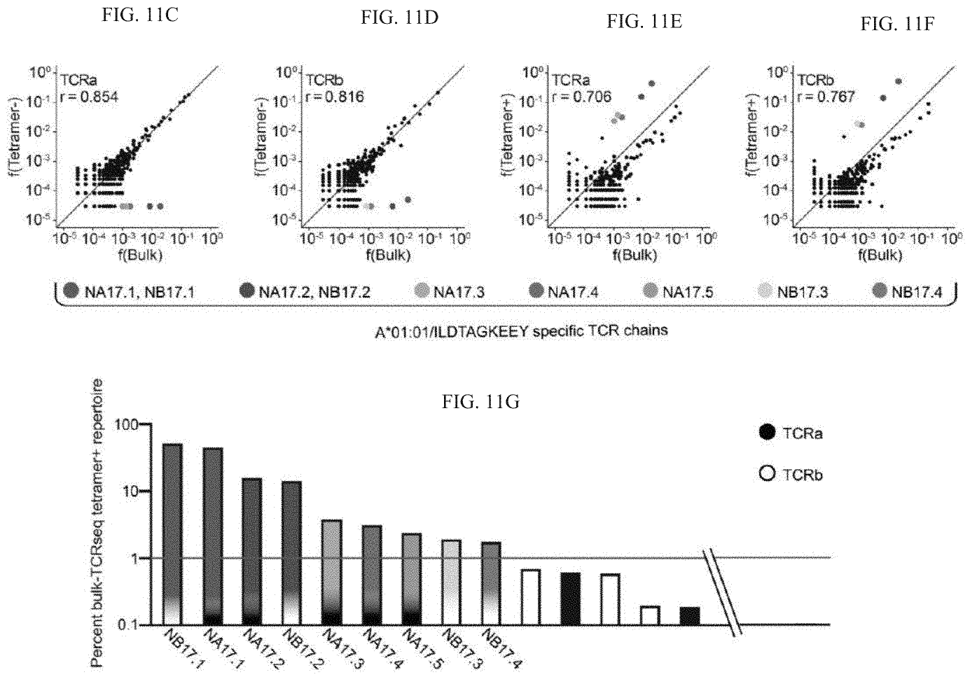

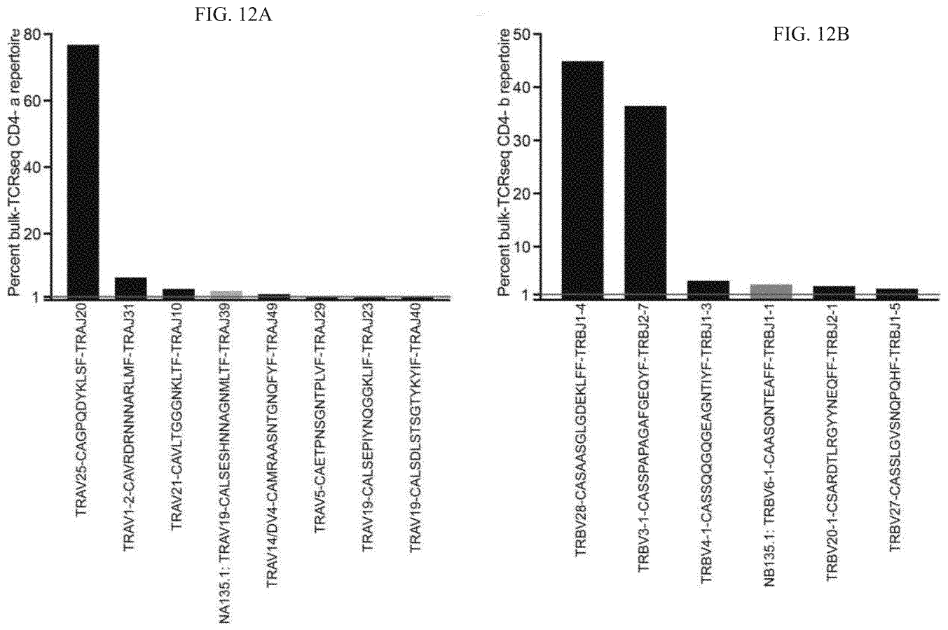

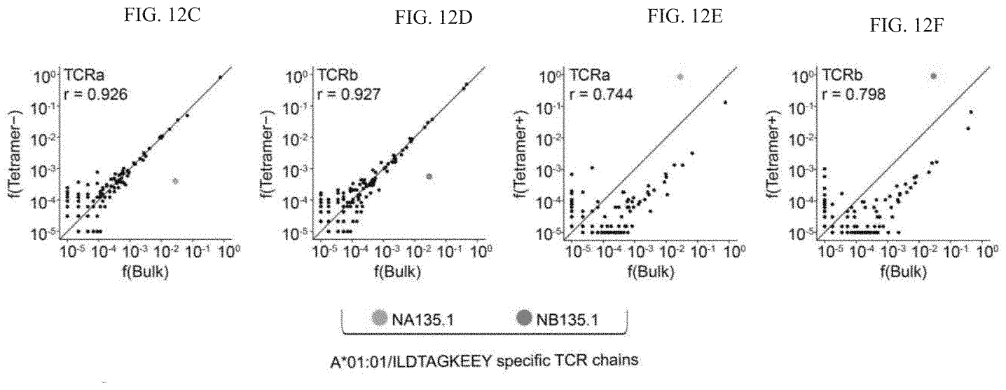

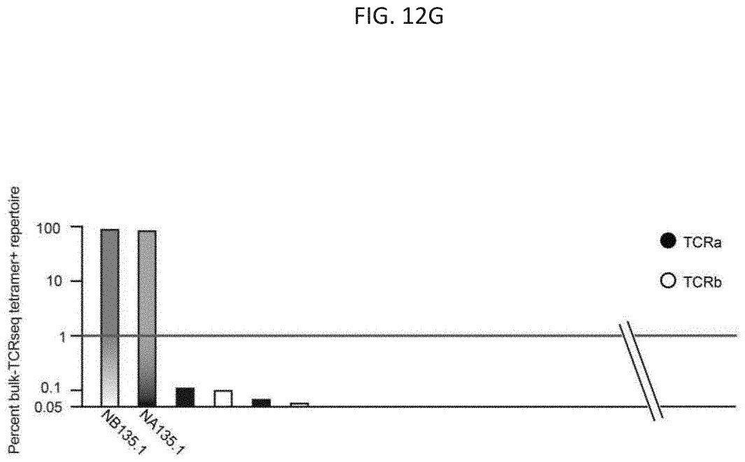

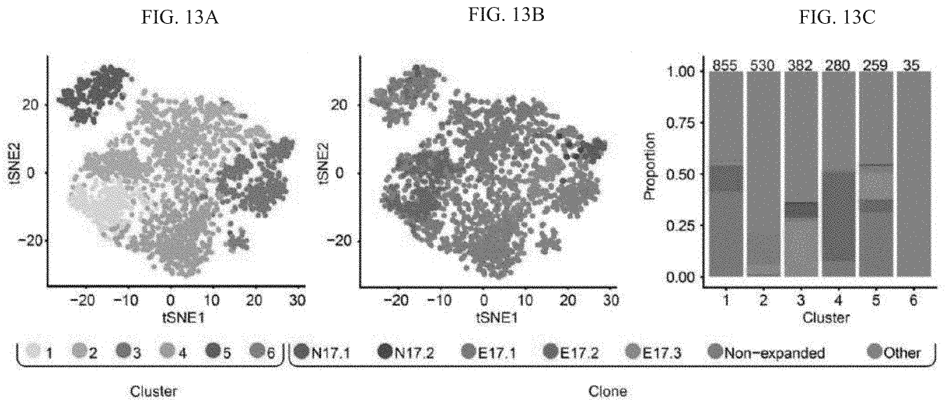

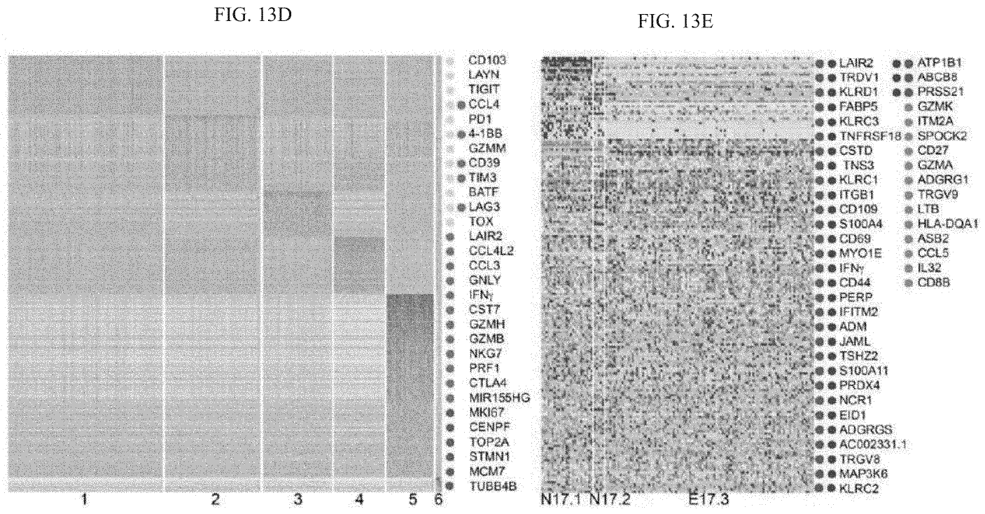

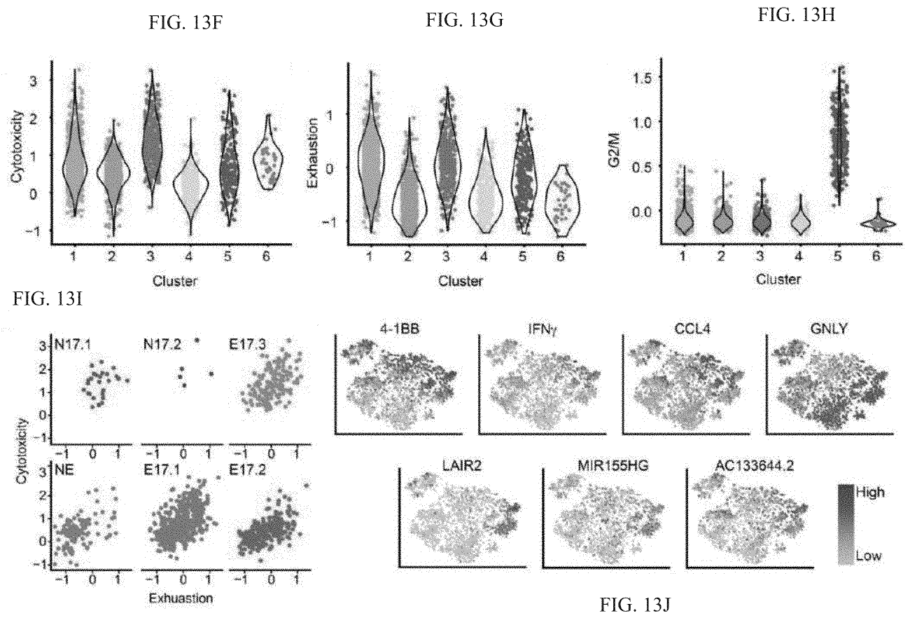

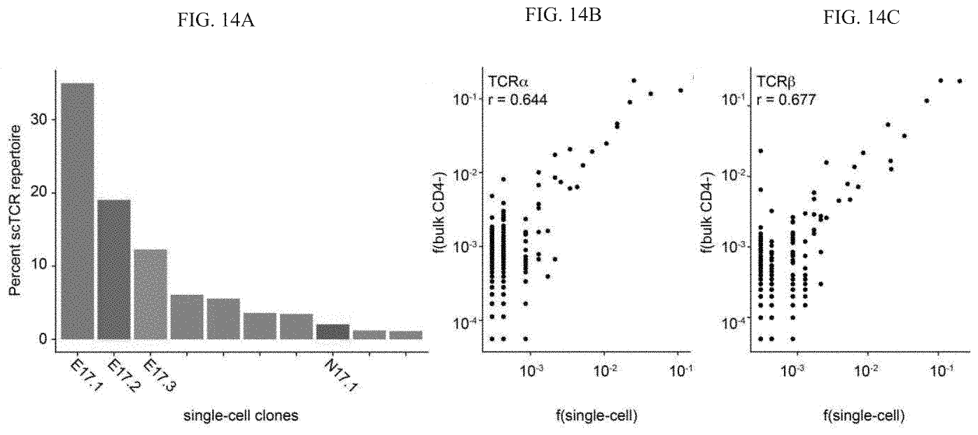

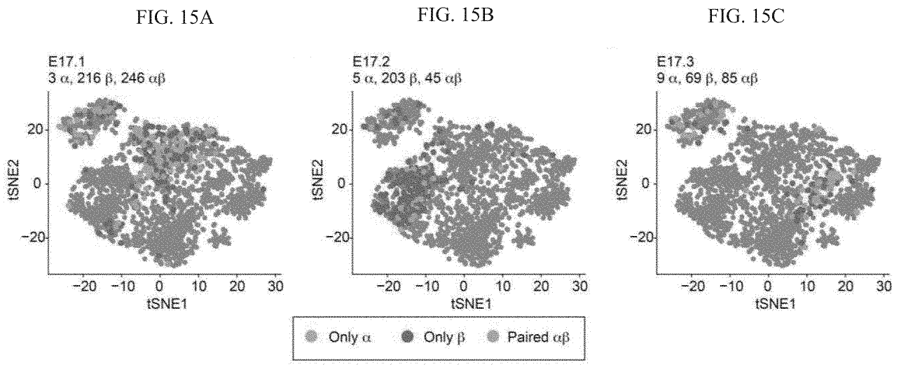

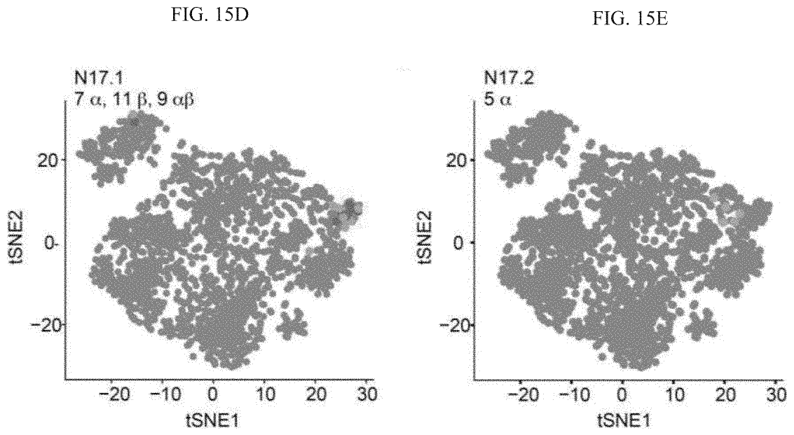

1. Tran E, Robbins P F, Rosenberg S A. “Final common pathway” of human cancer immunotherapy: targeting random somatic mutations. Nat Immunol. 2017; 18(3):255-262. doi:10.1038/ni.3682
2. Gubin M M, Zhang X, Schuster H, et al. Checkpoint blockade cancer immunotherapy targets tumour-specific mutant antigens. Nature. 2014; 515(7528):577-581. doi:10.1038/nature13988
3. Tumeh P C, Harview C L, Yearley J H, et al. PD-1 blockade induces responses by inhibiting adaptive immune resistance. Nature. 2014; 515(7528):568-571. doi:10.1038/nature13954
4. Robbins P F, Lu Y-C, El-Gamil M, et al. Mining exomic sequencing data to identify mutated antigens recognized by adoptively transferred tumor-reactive T cells. Nat Med. 2013; 19(6):747-752. www(dot)ncbi(dot)nlm(dotnih(dot)gov/pubmed/23644516. Accessed Mar. 24, 2018.
5. Lu Y-C, Yao X, Crystal J S, et al. Efficient Identification of Mutated Cancer Antigens Recognized by T Cells Associated with Durable Tumor Regressions. Clin Cancer Res. 2014; 20(13):3401-3410. doi:10.1158/1078-0432.CCR-14-0433
6. Tran E, Turcotte S, Gros A, Robbins P, . . . Y L-, 2014 undefined. Cancer immunotherapy based on mutation-specific CD4+ T cells in a patient with epithelial cancer. science(dot)sciencemag(dot)org. science(dot)sciencemag(dot)org/content/344/6184/641. short. Accessed Mar. 21, 2018.
7. Tran E, Robbins P F, Lu Y-C, et al. T-Cell Transfer Therapy Targeting Mutant KRAS in Cancer. N Engl J Med. 2016; 375(23):2255-2262. doi:10.1056/NEJMoa1609279
8. Rosenberg S A. Raising the Bar: The Curative Potential of Human Cancer Immunotherapy. Sci Transl Med. 2012; 4(127):127 ps8. doi:10.1126/scitranslmed.3003634
9. Ott P A, Hu Z, Keskin D B, et al. An immunogenic personal neoantigen vaccine for patients with melanoma. Nature. 2017; 547(7662):217-221. doi:10.1038/nature22991
10. Sahin U, Derhovanessian E, Miller M, et al. Personalized RNA mutanome vaccines mobilize poly-specific therapeutic immunity against cancer. Nature. 2017; 547(7662):222-226. doi:10.1038/nature23003
11. Carreno B M, Magrini V, Becker-Hapak M, et al. A dendritic cell vaccine increases the breadth and diversity of melanoma neoantigen-specific T cells. Science (80-). 2015; 348(6236):803-808. doi:10.1126/science.aaa3828
12. Kalaora S, Barnea E, Merhavi-Shoham E, et al. Use of HLA peptidomics and whole exome sequencing to identify human immunogenic neo-antigens. Oncotarget. 2016; 7(5):5110-5117. doi:10.18632/oncotarget.6960
13. Kalaora S, Wolf Y, Feferman T, et al. Combined Analysis of Antigen Presentation and T-cell Recognition Reveals Restricted Immune Responses in Melanoma. Cancer Discov. 2018; 8(11):1366-1375. doi:10.1158/2159-8290.CD-17-1418
14. Carbone D P, Ciernik I F, Kelley M J, et al. Immunization with mutant p53- and K-ras-derived peptides in cancer patients: immune response and clinical outcome. J Clin Oncol. 2005; 23(22):5099-5107. doi:10.1200/JCO.2005.03.158
15. Strønen E, Toebes M, Kelderman S, et al. Targeting of cancer neoantigens with donor-derived T cell receptor repertoires. Science. 2016; 352(6291): 1337-1341. doi:10.1126/science.aaf2288
16. Linard B, Bézieau S, Benlalam H, et al. A ras-mutated peptide targeted by CTL infiltrating a human melanoma lesion. J Immunol. 2002; 168(9):4802-4808. doi:10.4049/JIMMUNOL.168.9.4802
17. Iii T G-D, Spurkland A, Fossum B, Thorsby E, Gaudernack G, Wittinghofer A. T cell epitopes encompassing the mutational hot spot position 61 of p21 ras. Promiscuity in ras peptide binding to HLA. Eur J Immunol. 1994; 24(2):410-414. doi:10.1002/eji.1830240221
18. Sharkey M S, Lizée G, Gonzales M I, Patel S, Topalian S L. CD4(+) T-cell recognition of mutated B-RAF in melanoma patients harboring the V599E mutation. Cancer Res. 2004; 64(5): 1595-1599. www(dot)ncbi(dot)nlm(dot)nih(dot)gov/pubmed/14996715. Accessed Mar. 25, 2018.
19. Shamalov K, Levy S N, Horovitz-Fried M, Cohen C J. The mutational status of p53 can influence its recognition by human T-cells. Oncoimmunology. 2017; 6(4):e1285990. doi: 10.1080/2162402X.2017.0.1285990
20. Tran E, Ahmadzadeh M, Lu Y-C, et al. Immunogenicity of somatic mutations in human gastrointestinal cancers. Science. 2015; 350(6266): 1387-1390. doi:10.1126/science.aad1253
21. Tran E, Robbins P F, Lu Y-C, et al. T-Cell Transfer Therapy Targeting Mutant KRAS in Cancer. N Engl J Med. 2016; 375(23):2255-2262. doi:10.1056/NEJMoa1609279
22. Malekzadeh P, Pasetto A, Robbins P F, et al. Neoantigen screening identifies broad TP53 mutant immunogenicity in patients with epithelial cancers. J Clin Invest. 2019; 129(3):1109-1114. doi:10.1172/JC1123791
23. Deniger D C, Pasetto A, Robbins P F, et al. T-cell Responses to TP53 “Hotspot” Mutations and Unique Neoantigens Expressed by Human Ovarian Cancers. Clin Cancer Res. 2018; 24(22):5562-5573. doi:10.1158/1078-0432.CCR-18-0573
24. Cafri G, Yossef R, Pasetto A, et al. Memory T cells targeting oncogenic mutations detected in peripheral blood of epithelial cancer patients. Nat Commun. 2019; 10(1):449. doi:10.1038/s41467-019-08304-z
25. Iiizumi S, Ohtake J, Murakami N, et al. Identification of Novel HLA Class II-Restricted Neoantigens Derived from Driver Mutations. Cancers (Basel). 2019; 11(2):266. doi:10.3390/cancers11020266
26. Lawrence M S, Stojanov P, Polak P, et al. Mutational heterogeneity in cancer and the search for new cancer-associated genes. Nature. 2013; 499(7457):214-218. doi:10.1038/nature12213
27. Marty R, Kaabinejadian S, Rossell D, et al. MHC-I Genotype Restricts the Oncogenic Mutational Landscape. Cell. 2017; 171(6): 1272-1283.e15. doi: 10.1016/j.cell.2017.0.09.050
28. Simanshu D K, Nissley D V., McCormick F. RAS Proteins and Their Regulators in Human Disease. Cell. 2017; 170(1): 17-33. doi: 10.1016/j.cell.2017.0.06.009
29. Johnson D B, Lovly C M, Flavin M, et al. Impact of NRAS Mutations for Patients with Advanced Melanoma Treated with Immune Therapies. Cancer Immunol Res. 2015; 3(3):288-295. doi:10.1158/2326-6066.CIR-14-0207
30. Jurtz V, Paul S, Andreatta M, Marcatili P, Peters B, Nielsen M. NetMHCpan-4.0: Improved Peptide-MHC Class I Interaction Predictions Integrating Eluted Ligand and Peptide Binding Affinity Data. J Immunol. 2017; 199(9):3360-3368. doi:10.4049/jimmunol.1700893
31. Fritsch E F, Rajasagi M, Ott P A, Brusic V, Hacohen N, Wu C J. Cancer Immunology Miniatures HLA-Binding Properties of Tumor Neoepitopes in Humans. 2014. doi:10.1158/2326-6066.CIR-13-0227
32. Arafeh R, Qutob N, Emmanuel R, et al. Recurrent inactivating RASA2 mutations in melanoma. Nat Genet. 2015; 47(12): 1408-1410. doi:10.1038/ng.3427
33. Cox J, Mann M. MaxQuant enables high peptide identification rates, individualized p.p.b.-range mass accuracies and proteome-wide protein quantification. Nat Biotechnol. 2008; 26(12):1367-1372. doi:10.1038/nbt.1511
34. Toor J S, Rao A A, McShan A C, et al. A Recurrent Mutation in Anaplastic Lymphoma Kinase with Distinct Neoepitope Conformations. Front Immunol. 2018; 9:99. doi:10.3389/fimmu.2018.00099
35. London N, Raveh B, Cohen E, Fathi G, Schueler-Furman O. Rosetta FlexPepDock web server—high resolution modeling of peptide-protein interactions. Nucleic Acids Res. 2011; 39(Web Server issue):W249-53. doi:10.1093/nar/gkr431
36. Vajda S, Yueh C, Beglov D, et al. New Additions to the ClusPro Server Motivated by CAPRI. doi:10.1002/prot.25219
37. Antunes D A, Moll M, Devaurs D, Jackson K R, Lizee G, Kavraki L E. DINC 2.0: A New Protein—Peptide Docking Webserver Using an Incremental Approach. Cancer Res. 2017; 77(21):e55-e57. doi:10.1158/0008-5472.CAN-17-0511
38. IHWG FRED HUTCH Cell Lines &amp; Genes. www(dot)fredhutch(dot)org/en/labs/clinical/projects/ihwg/cell-lines-genes(dot)html. Accessed Mar. 4, 2018.
39. Giam K, Ayala-Perez R, Illing P T, et al. A comprehensive analysis of peptides presented by HLA-A1. Tissue Antigens. 2015; 85(6):492-496. doi:10.1111/tan.12565
40. Riaz N, Havel J J, Makarov V, et al. Tumor and Microenvironment Evolution during Immunotherapy with Nivolumab. Cell. 2017; 171(4):934-949.e16. doi: 10.1016/J.CELL.2017.0.09.028
41. Lu Y-C, Yao X, Li Y F, et al. Mutated PPP1R3B is recognized by T cells used to treat a melanoma patient who experienced a durable complete tumor regression. J Immunol. 2013; 190(12):6034-6042. doi:10.4049/jimmuno1.1202830
42. Wei X, Walia V, Lin J C, et al. Exome sequencing identifies GRIN2A as frequently mutated in melanoma. Nat Genet. 2011; 43(5):442-446. doi: 10.1038/ng 0.810
43. Klapper J A, Thomasian A A, Smith D M, et al. Single-pass, closed-system rapid expansion of lymphocyte cultures for adoptive cell therapy. J Immunol Methods. 2009; 345(1-2):90-99. doi:10.1016/j.jim.2009.04.009
44. ATCC.
45. DSMZ.
46. Shukla S A, Rooney M S, Rajasagi M, et al. Comprehensive analysis of cancer-associated somatic mutations in class I HLA genes. Nat Biotechnol. 2015; 33(11):1152-1158. doi:10.1038/nbt.3344
47. Scholtalbers J, Boegel S, Bukur T, et al. TCLP: an online cancer cell line catalogue integrating HLA type, predicted neo-epitopes, virus and gene expression. Genome Med. 2015; 7(1):118. doi:10.1186/s13073-015-0240-5
48. Marty R, Kaabinejadian S, Rossell D, Hildebrand W H, Font-Burgada J, Correspondence H C. MHC-I Genotype Restricts the Oncogenic Mutational Landscape In Brief HLA genotype-restricted immunoediting during tumor formation shapes the landscape of oncogenic mutations observed in clinically diagnosed tumors. Cell. 2017; 171. doi:10.1016/j.cell.2017.0.09.050
49. Cerami E, Gao J, Dogrusoz U, et al. The cBio Cancer Genomics Portal: An Open Platform for Exploring Multidimensional Cancer Genomics Data: FIG. 1. Cancer Discov. 2012; 2(5):401-404. doi:10.1158/2159-8290.CD-12-0095
50. Gao J, Aksoy B A, Dogrusoz U, et al. Integrative Analysis of Complex Cancer Genomics and Clinical Profiles Using the cBioPortal. Sci Signal. 2013; 6(269):p 11-p 11. doi:10.1126/scisignal.2004088
51. The Cancer Genome Atlas Program-National Cancer Institute. www(dot)cancer(dot)gov/about-nci/organization/ccg/research/structural-genomics/tcga. Accessed Mar. 21, 2019.
52. Páll S, Abraham M J, Kutzner C, Hess B, Lindahl E. Tackling Exascale Software Challenges in Molecular Dynamics Simulations with GROMACS. In: Springer, Cham; 2015:3-27. doi:10.1007/978-3-319-15976-8_1
53. Schmid N, Eichenberger A P, Choutko A, et al. Definition and testing of the GROMOS force-field versions 54A7 and 54B7. Eur Biophys J. 2011; 40(7):843-856. doi:10.1007/s00249-011-0700-9
54. Hess B, Bekker H, Berendsen H J C, Fraaije JGEM. LINCS: A linear constraint solver for molecular simulations. J Comput Chem. 1997; 18(12): 1463-1472. doi:10.1002/(SICI)1096-987X(199709)18:12<1463::AID-JCC4>3.0.00; 2-H
55. Bussi G, Donadio D, Parrinello M. Canonical sampling through velocity rescaling. J Chem Phys. 2007; 126(1):014101. doi:10.1063/1.2408420
56. Parrinello M, Rahman A. Polymorphic transitions in single crystals: A new molecular dynamics method. J Appl Phys. 1981; 52(12):7182-7190. doi:10.1063/1.328693
57. Essmann U, Perera L, Berkowitz M L, Darden T, Lee H, Pedersen L G. A smooth particle mesh Ewald method. J Chem Phys. 1995; 103(19):8577-8593. doi:10.1063/1.470117
58. Schrodinger L L C. The PyMOL Molecular Graphics System, Version 1.8. 2015.
59. Wernet P, Nordlund D, Bergmann U, et al. The Structure of the First Coordination Shell in Liquid Water. Science (80-). 2004; 304(5673):995 LP-999. doi:10.1126/science.1096205
60. McGibbon R T, Beauchamp K A, Harrigan M P, et al. MDTraj: A Modern Open Library for the Analysis of Molecular Dynamics Trajectories. Biophys J. 2015; 109(8):1528-1532. doi:10.1016/J.BPJ.2015.08.015
61. Milner E, Gutter-Kapon L, Bassani-Strenberg M, Barnea E, Beer I, Admon A. The Effect of Proteasome Inhibition on the Generation of the Human Leukocyte Antigen (HLA) Peptidome. Mol Cell Proteomics. 2013; 12(7):1853-1864. doi:10.1074/mcp.M112.026013
62. Bassani-Sternberg M, Barnea E, Beer I, Avivi I, Katz T, Admon A. Soluble plasma HLA peptidome as a potential source for cancer biomarkers. Proc Natl Acad Sci. 2010; 107(44):18769-18776. doi:10.1073/pnas 0.1008501107
63. Rappsilber J, Ishihama Y, Mann M. Stop and go extraction tips for matrix-assisted laser desorption/ionization, nanoelectrospray, and LC/MS sample pretreatment in proteomics. Anal Chem. 2003; 75(3):663-670. www(dot)ncbi(dot)nlm(dot)nih(dot)gov/pubmed/12585499. Accessed Mar. 5, 2019.
64. Ishihama Y, Rappsilber J, Andersen J S, Mann M. Microcolumns with self-assembled particle frits for proteomics. J Chromatogr A. 2002; 979(1-2):233-239. www(dot)ncbi(dot)nlm(dot)nih(dot)gov/pubmed/12498253. Accessed Mar. 5, 2019.
65. UniProt: a worldwide hub of protein knowledge. Nucleic Acids Res. 2019; 47(D1):D506-D515. doi:10.1093/nar/gky1049
66. Cox J, Neuhauser N, Michalski A, Scheltema R A, Olsen J V., Mann M. Andromeda: A Peptide Search Engine Integrated into the MaxQuant Environment. J Proteome Res. 2011; 10(4):1794-1805. doi:10.1021/pr101065j
67. Shelly Kalaora, Yochai Wolf, Tali Feferman, Eilon Barnea, Erez Greenstein, Dan Reshef, Itay Tirosh, Alexandre Reuben, Ronen Levy, Juliane Quinkhardt, Tana Omokoko, Nouar Qutob, Ofra Golani, Chantale Bernatchez, Cara Haymaker, Marie-Andrée F Y S. Combined analysis of antigen presentation and T cell recognition reveals restricted immune responses in melanoma. Cancer Discov. 2018.
68. Bosselut R, Huseby E, Oakes T, et al. Quantitative Characterization of the t Cell Receptor Repertoire of Naïve and Memory subsets Using an Integrated experimental and Computational Pipeline Which Is Robust, economical, and Versatile. Immunol. 2017; 8:1267. doi:10.3389/fimmu.2017.01267
69. Greiff V, Miho E, Menzel U, Reddy S T. Bioinformatic and Statistical Analysis of Adaptive Immune Repertoires. Trends Immunol. 2015; 36(11):738-749. doi:10.1016/j.it.2015.09.006

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of treating cancer in a subject comprising administering to the subject a therapeutically effective amount of T cells expressing a T cell receptor (TCR) having a CDR3 amino acid sequence selected from the group consisting of 199-210, thereby treating the cancer of the subject.

2. The method of claim 1, wherein said TCR binds to a peptide having a sequence as set forth in SEQ ID NO: 1 in a complex with HLA-A*01:01 allele in the subject.

3. The method of claim 1, wherein said T cells are autologous to the subject.

4. The method of claim 1, wherein said T cells are non-autologous to the subject.

5. The method of claim 1, wherein said T cells are genetically modified to express said T cell receptor.

6. The method of claim 1, wherein said T cells comprise CD8+ T cells.

7. The method of claim 1, wherein said cancer is selected from the group consisting of melanoma, colon cancer, breast cancer, thyroid cancer, stomach cancer, colorectal cancer, leukemia cancer, bladder cancer, lung cancer, ovarian cancer, breast cancer and prostate cancer.

8. The method of claim 1, wherein said cancer is melanoma.

9. An isolated population of T cells genetically modified to express a T cell receptor (TCR) having a CDR3 amino acid sequence selected from the group consisting of 199-210.

10. The isolated population of T cells of claim 9, being CD8+ T cells.

11-35. (canceled)

36. A method of treating cancer of a subject comprising:
(a) ascertaining the HLA profile of a subject;
(b) determining whether the subject expresses NRAS.Q61K; and
(c) when the subject has been identified as being HLA-A*01:01/NRAS.Q61K, treating said subject with a therapeutically effective amount of an agent that targets the peptide having an amino acid sequence as set forth in SEQ ID NO: 1, thereby treating the cancer.

37. A method of treating cancer of a subject comprising:
(a) ascertaining the HLA profile of a subject;
(b) determining whether the subject expresses a RAS variant selected from the group consisting of Q61K, Q61R, Q61L and Q61H; and
(c) when the subject expresses said RAS variant, treating said subject with a therapeutically effective amount of an agent that targets a peptide having an amino acid sequence selected from the group consisting of SEQ ID NOs: 1 and 12-132, wherein the peptide is selected according to the corresponding HLA profile as set forth in Table 1C.

38-39. (canceled)

40. The method of claim 37, wherein said peptide has an amino acid sequence selected from the group consisting of SEQ ID NOs: 1 and 12-28.

41. The method of claim 37, wherein said RAS variant is NRAS.

42. The method of claim 36, wherein said cancer is selected from the group consisting of melanoma, colon cancer, breast cancer, thyroid cancer, stomach cancer, colorectal cancer, leukemia cancer, bladder cancer, lung cancer, ovarian cancer, breast cancer and prostate cancer.

43. The method of claim 36, wherein said agent is selected from the group consisting of a vaccine, an antibody and a population of T cells expressing a receptor that targets said T cell epitope.

44. The method of claim 36, further comprising treating the subject with a checkpoint inhibitor.


SEQUENCE LISTING (247 sequences)

SEQ ID NO: 1 - protein, 10 aa, Artificial sequence
  synthetic peptide
ILDTAGKEEY

SEQ ID NO: 2 - protein, 10 aa, Artificial sequence
  synthetic peptide
ILDTAGQEEY

SEQ ID NO: 3 - DNA, 20 bp, Artificial sequence
  Single strand DNA oligonucleotide
ttggagcaggtggtgttggg

SEQ ID NO: 4 - DNA, 21 bp, Artificial sequence
  Single strand DNA oligonucleotide
gtatcaactgtccttgttggc

SEQ ID NO: 5 - protein, 27 aa, Artificial sequence
  synthetic peptide
GETCLLDILDTAGQEEYSAMRDQYMRT

SEQ ID NO: 6 - protein, 27 aa, Artificial sequence
  synthetic peptide
GETCLLDILDTAGREEYSAMRDQYMRT

SEQ ID NO: 7 - protein, 27 aa, Artificial sequence
  synthetic peptide
GETCLLDILDTAGKEEYSAMRDQYMRT

SEQ ID NO: 8 - protein, 27 aa, Artificial sequence
  synthetic peptide
GETCLLDILDTAGLEEYSAMRDQYMRT

SEQ ID NO: 9 - protein, 27 aa, Artificial sequence
  synthetic peptide
GETCLLDILDTAGHEEYSAMRDQYMRT

SEQ ID NO: 10 - protein, 11 aa, Artificial sequence
  synthetic peptide
CATDCKNQFYF

SEQ ID NO: 11 - protein, 13 aa, Artificial sequence
  synthetic peptide
CASEEGGGFKTIF

SEQ ID NO: 12 - protein, 10 aa, Artificial sequence
  synthetic peptide
ILDTAGLEEY

SEQ ID NO: 13 - protein, 10 aa, Artificial sequence
  synthetic peptide
ILDTAGHEEY

SEQ ID NO: 14 - protein, 13 aa, Artificial sequence
  synthetic peptide
LLDILDTAGHEEY

SEQ ID NO: 15 - protein, 13 aa, Artificial sequence
  synthetic peptide
LLDILDTAGLEEY

SEQ ID NO: 16 - protein, 10 aa, Artificial sequence
  synthetic peptide
ILDTAGREEY

SEQ ID NO: 17 - protein, 13 aa, Artificial sequence
  synthetic peptide
LLDILDTAGREEY

SEQ ID NO: 18 - protein, 13 aa, Artificial sequence
  synthetic peptide
LLDILDTAGKEEY

SEQ ID NO: 19 - protein, 11 aa, Artificial sequence
  synthetic peptide
DTAGHEEYSAM

SEQ ID NO: 20 - protein, 11 aa, Artificial sequence
  synthetic peptide
DTAGLEEYSAM

SEQ ID NO: 21 - protein, 11 aa, Artificial sequence
  synthetic peptide
DTAGREEYSAM

SEQ ID NO: 22 - protein, 11 aa, Artificial sequence
  synthetic peptide
DTAGHEEYSAM

SEQ ID NO: 23 - protein, 11 aa, Artificial sequence
  synthetic peptide
DTAGLEEYSAM

SEQ ID NO: 24 - protein, 11 aa, Artificial sequence
  synthetic peptide
DTAGKEEYSAM

SEQ ID NO: 25 - protein, 10 aa, Artificial sequence
  synthetic peptide
LLDILDTAGL

SEQ ID NO: 26 - protein, 11 aa, Artificial sequence
  synthetic peptide
DTAGREEYSAM

SEQ ID NO: 27 - protein, 10 aa, Artificial sequence
  synthetic peptide
LLDILDTAGL

SEQ ID NO: 28 - protein, 11 aa, Artificial sequence
  synthetic peptide
DTAGKEEYSAM

SEQ ID NO: 29 - protein, 11 aa, Artificial sequence
  synthetic peptide
DILDTAGLEEY

SEQ ID NO: 30 - protein, 11 aa, Artificial sequence
  synthetic peptide
DILDTAGLEEY

SEQ ID NO: 31 - protein, 8 aa, Artificial sequence
  synthetic peptide
DTAGLEEY

SEQ ID NO: 32 - protein, 13 aa, Artificial sequence
  synthetic peptide
ETCLLDILDTAGR

SEQ ID NO: 33 - protein, 12 aa, Artificial sequence
  synthetic peptide
DTAGLEEYSAMR

SEQ ID NO: 34 - protein, 14 aa, Artificial sequence
  synthetic peptide
GETCLLDILDTAGL

SEQ ID NO: 35 - protein, 13 aa, Artificial sequence
  synthetic peptide
ILDTAGLEEYSAM

SEQ ID NO: 36 - protein, 11 aa, Artificial sequence
  synthetic peptide
REEYSAMRDQY

SEQ ID NO: 37 - protein, 11 aa, Artificial sequence
  synthetic peptide
KEEYSAMRDQY

SEQ ID NO: 38 - protein, 14 aa, Artificial sequence
  synthetic peptide
CLLDILDTAGHEEY

SEQ ID NO: 39 - protein, 14 aa, Artificial sequence
  synthetic peptide
CLLDILDTAGLEEY

SEQ ID NO: 40 - protein, 11 aa, Artificial sequence
  synthetic peptide
HEEYSAMRDQY

SEQ ID NO: 41 - protein, 12 aa, Artificial sequence
  synthetic peptide
DTAGHEEYSAMR

SEQ ID NO: 42 - protein, 11 aa, Artificial sequence
  synthetic peptide
DILDTAGREEY

SEQ ID NO: 43 - protein, 11 aa, Artificial sequence
  synthetic peptide
LEEYSAMRDQY

SEQ ID NO: 44 - protein, 11 aa, Artificial sequence
  synthetic peptide
DILDTAGHEEY

SEQ ID NO: 45 - protein, 13 aa, Artificial sequence
  synthetic peptide
ILDTAGHEEYSAM

SEQ ID NO: 46 - protein, 14 aa, Artificial sequence
  synthetic peptide
CLLDILDTAGREEY

SEQ ID NO: 47 - protein, 11 aa, Artificial sequence
  synthetic peptide
HEEYSAMRDQY

SEQ ID NO: 48 - protein, 10 aa, Artificial sequence
  synthetic peptide
ILDTAGLEEY

SEQ ID NO: 49 - protein, 11 aa, Artificial sequence
  synthetic peptide
DILDTAGHEEY

SEQ ID NO: 50 - protein, 14 aa, Artificial sequence
  synthetic peptide
CLLDILDTAGKEEY

SEQ ID NO: 51 - protein, 11 aa, Artificial sequence
  synthetic peptide
DILDTAGKEEY

SEQ ID NO: 52 - protein, 12 aa, Artificial sequence
  synthetic peptide
DTAGKEEYSAMR

SEQ ID NO: 53 - protein, 12 aa, Artificial sequence
  synthetic peptide
DTAGREEYSAMR

SEQ ID NO: 54 - protein, 9 aa, Artificial sequence
  synthetic peptide
LDILDTAGL

SEQ ID NO: 55 - protein, 11 aa, Artificial sequence
  synthetic peptide
REEYSAMRDQY

SEQ ID NO: 56 - protein, 8 aa, Artificial sequence
  synthetic peptide
DTAGHEEY

SEQ ID NO: 57 - protein, 12 aa, Artificial sequence
  synthetic peptide
GLEEYSAMRDQY

SEQ ID NO: 58 - protein, 11 aa, Artificial sequence
  synthetic peptide
LEEYSAMRDQY

SEQ ID NO: 59 - protein, 10 aa, Artificial sequence
  synthetic peptide
ILDTAGHEEY

SEQ ID NO: 60 - protein, 11 aa, Artificial sequence
  synthetic peptide
KEEYSAMRDQY

SEQ ID NO: 61 - protein, 12 aa, Artificial sequence
  synthetic peptide
LDILDTAGLEEY

SEQ ID NO: 62 - protein, 10 aa, Artificial sequence
  synthetic peptide
LLDILDTAGK

SEQ ID NO: 63 - protein, 13 aa, Artificial sequence
  synthetic peptide
ETCLLDILDTAGK

SEQ ID NO: 64 - protein, 9 aa, Artificial sequence
  synthetic peptide
AGHEEYSAM

SEQ ID NO: 65 - protein, 11 aa, Artificial sequence
  synthetic peptide
HEEYSAMRDQY

SEQ ID NO: 66 - protein, 13 aa, Artificial sequence
  synthetic peptide
ILDTAGLEEYSAM

SEQ ID NO: 67 - protein, 13 aa, Artificial sequence
  synthetic peptide
ILDTAGREEYSAM

SEQ ID NO: 68 - protein, 13 aa, Artificial sequence
  synthetic peptide
ILDTAGKEEYSAM

SEQ ID NO: 69 - protein, 11 aa, Artificial sequence
  synthetic peptide
ILDTAGLEEYS

SEQ ID NO: 70 - protein, 10 aa, Artificial sequence
  synthetic peptide
ILDTAGHEEY

SEQ ID NO: 71 - protein, 13 aa, Artificial sequence
  synthetic peptide
ILDTAGHEEYSAM

SEQ ID NO: 72 - protein, 11 aa, Artificial sequence
  synthetic peptide
LEEYSAMRDQY

SEQ ID NO: 73 - protein, 9 aa, Artificial sequence
  synthetic peptide
AGLEEYSAM

SEQ ID NO: 74 - protein, 9 aa, Artificial sequence
  synthetic peptide
AGLEEYSAM

SEQ ID NO: 75 - protein, 13 aa, Artificial sequence
  synthetic peptide
ETCLLDILDTAGL

SEQ ID NO: 76 - protein, 9 aa, Artificial sequence
  synthetic peptide
AGHEEYSAM

SEQ ID NO: 77 - protein, 9 aa, Artificial sequence
  synthetic peptide
AGHEEYSAM

SEQ ID NO: 78 - protein, 11 aa, Artificial sequence
  synthetic peptide
REEYSAMRDQY

SEQ ID NO: 79 - protein, 10 aa, Artificial sequence
  synthetic peptide
LLDILDTAGL

SEQ ID NO: 80 - protein, 11 aa, Artificial sequence
  synthetic peptide
CLLDILDTAGL

SEQ ID NO: 81 - protein, 9 aa, Artificial sequence
  synthetic peptide
AGHEEYSAM

SEQ ID NO: 82 - protein, 8 aa, Artificial sequence
  synthetic peptide
DTAGLEEY

SEQ ID NO: 83 - protein, 9 aa, Artificial sequence
  synthetic peptide
AGLEEYSAM

SEQ ID NO: 84 - protein, 11 aa, Artificial sequence
  synthetic peptide
KEEYSAMRDQY

SEQ ID NO: 85 - protein, 9 aa, Artificial sequence
  synthetic peptide
AGREEYSAM

SEQ ID NO: 86 - protein, 11 aa, Artificial sequence
  synthetic peptide
DILDTAGREEY

SEQ ID NO: 87 - protein, 8 aa, Artificial sequence
  synthetic peptide
DTAGREEY

SEQ ID NO: 88 - protein, 13 aa, Artificial sequence
  synthetic peptide
ILDTAGREEYSAM

SEQ ID NO: 89 - protein, 10 aa, Artificial sequence
  synthetic peptide
ILDTAGLEEY

SEQ ID NO: 90 - protein, 13 aa, Artificial sequence
  synthetic peptide
ILDTAGKEEYSAM

SEQ ID NO: 91 - protein, 12 aa, Artificial sequence
  synthetic peptide
LDILDTAGHEEY

SEQ ID NO: 92 - protein, 11 aa, Artificial sequence
  synthetic peptide
DILDTAGKEEY

SEQ ID NO: 93 - protein, 10 aa, Artificial sequence
  synthetic peptide
AGREEYSAMR

SEQ ID NO: 94 - protein, 10 aa, Artificial sequence
  synthetic peptide
ILDTAGREEY

SEQ ID NO: 95 - protein, 14 aa, Artificial sequence
  synthetic peptide
GREEYSAMRDQYMR

SEQ ID NO: 96 - protein, 11 aa, Artificial sequence
  synthetic peptide
CLLDILDTAGK

SEQ ID NO: 97 - protein, 10 aa, Artificial sequence
  synthetic peptide
ILDTAGHEEY

SEQ ID NO: 98 - protein, 10 aa, Artificial sequence
  synthetic peptide
ILDTAGLEEY

SEQ ID NO: 99 - protein, 9 aa, Artificial sequence
  synthetic peptide
AGREEYSAM

SEQ ID NO: 100 - protein, 13 aa, Artificial sequence
  synthetic peptide
ETCLLDILDTAGL

SEQ ID NO: 101 - protein, 11 aa, Artificial sequence
  synthetic peptide
ILDTAGHEEYS

SEQ ID NO: 102 - protein, 9 aa, Artificial sequence
  synthetic peptide
AGHEEYSAM

SEQ ID NO: 103 - protein, 14 aa, Artificial sequence
  synthetic peptide
LLDILDTAGHEEYS

SEQ ID NO: 104 - protein, 13 aa, Artificial sequence
  synthetic peptide
ETCLLDILDTAGR

SEQ ID NO: 105 - protein, 12 aa, Artificial sequence
  synthetic peptide
DTAGLEEYSAMR

SEQ ID NO: 106 - protein, 12 aa, Artificial sequence
  synthetic peptide
DTAGREEYSAMR

SEQ ID NO: 107 - protein, 10 aa, Artificial sequence
  synthetic peptide
ILDTAGREEY

SEQ ID NO: 108 - protein, 12 aa, Artificial sequence
  synthetic peptide
REEYSAMRDQYM

SEQ ID NO: 109 - protein, 14 aa, Artificial sequence
  synthetic peptide
DILDTAGHEEYSAM

SEQ ID NO: 110 - protein, 14 aa, Artificial sequence
  synthetic peptide
LLDILDTAGLEEYS

SEQ ID NO: 111 - protein, 10 aa, Artificial sequence
  synthetic peptide
TAGLEEYSAM

SEQ ID NO: 112 - protein, 8 aa, Artificial sequence
  synthetic peptide
DTAGHEEY

SEQ ID NO: 113 - protein, 11 aa, Artificial sequence
  synthetic peptide
REEYSAMRDQY

SEQ ID NO: 114 - protein, 11 aa, Artificial sequence
  synthetic peptide
DILDTAGLEEY

SEQ ID NO: 115 - protein, 14 aa, Artificial sequence
  synthetic peptide
DILDTAGKEEYSAM

SEQ ID NO: 116 - protein, 14 aa, Artificial sequence
  synthetic peptide
LLDILDTAGREEYS

SEQ ID NO: 117 - protein, 10 aa, Artificial sequence
  synthetic peptide
AGKEEYSAMR

SEQ ID NO: 118 - protein, 12 aa, Artificial sequence
  synthetic peptide
DTAGKEEYSAMR

SEQ ID NO: 119 - protein, 9 aa, Artificial sequence
  synthetic peptide
AGHEEYSAM

SEQ ID NO: 120 - protein, 14 aa, Artificial sequence
  synthetic peptide
LLDILDTAGKEEYS

SEQ ID NO: 121 - protein, 14 aa, Artificial sequence
  synthetic peptide
DILDTAGREEYSAM

SEQ ID NO: 122 - protein, 12 aa, Artificial sequence
  synthetic peptide
DTAGHEEYSAMR

SEQ ID NO: 123 - protein, 9 aa, Artificial sequence
  synthetic peptide
AGREEYSAM

SEQ ID NO: 124 - protein, 12 aa, Artificial sequence
  synthetic peptide
GREEYSAMRDQY

SEQ ID NO: 125 - protein, 13 aa, Artificial sequence
  synthetic peptide
HEEYSAMRDQYMR

SEQ ID NO: 126 - protein, 12 aa, Artificial sequence
  synthetic peptide
GKEEYSAMRDQY

SEQ ID NO: 127 - protein, 12 aa, Artificial sequence
  synthetic peptide
GHEEYSAMRDQY

SEQ ID NO: 128 - protein, 12 aa, Artificial sequence
  synthetic peptide
GLEEYSAMRDQY

SEQ ID NO: 129 - protein, 14 aa, Artificial sequence
  synthetic peptide
DILDTAGLEEYSAM

SEQ ID NO: 130 - protein, 13 aa, Artificial sequence
  synthetic peptide
REEYSAMRDQYMR

SEQ ID NO: 131 - protein, 10 aa, Artificial sequence
  synthetic peptide
ILDTAGKEEY

SEQ ID NO: 132 - protein, 9 aa, Artificial sequence
  synthetic peptide
AGREEYSAM

SEQ ID NO: 133 - protein, 11 aa, Artificial sequence
  synthetic peptide
DTAGQEEYSAM

SEQ ID NO: 134 - protein, 11 aa, Artificial sequence
  synthetic peptide
DTAGQEEYSAM

SEQ ID NO: 135 - protein, 13 aa, Artificial sequence
  synthetic peptide
LLDILDTAGQEEY

SEQ ID NO: 136 - protein, 14 aa, Artificial sequence
  synthetic peptide
CLLDILDTAGQEEY

SEQ ID NO: 137 - protein, 11 aa, Artificial sequence
  synthetic peptide
DILDTAGQEEY

SEQ ID NO: 138 - protein, 11 aa, Artificial sequence
  synthetic peptide
DILDTAGQEEY

SEQ ID NO: 139 - protein, 14 aa, Artificial sequence
  synthetic peptide
DILDTAGQEEYSAM

SEQ ID NO: 140 - protein, 8 aa, Artificial sequence
  synthetic peptide
DTAGQEEY

SEQ ID NO: 141 - protein, 12 aa, Artificial sequence
  synthetic peptide
DTAGQEEYSAMR

SEQ ID NO: 142 - protein, 12 aa, Artificial sequence
  synthetic peptide
DTAGQEEYSAMR

SEQ ID NO: 143 - protein, 12 aa, Artificial sequence
  synthetic peptide
GQEEYSAMRDQY

SEQ ID NO: 144 - protein, 10 aa, Artificial sequence
  synthetic peptide
ILDTAGQEEY

SEQ ID NO: 145 - protein, 10 aa, Artificial sequence
  synthetic peptide
ILDTAGQEEY

SEQ ID NO: 146 - protein, 13 aa, Artificial sequence
  synthetic peptide
ILDTAGQEEYSAM

SEQ ID NO: 147 - protein, 13 aa, Artificial sequence
  synthetic peptide
ILDTAGQEEYSAM

SEQ ID NO: 148 - protein, 12 aa, Artificial sequence
  synthetic peptide
LDILDTAGQEEY

SEQ ID NO: 149 - protein, 14 aa, Artificial sequence
  synthetic peptide
LLDILDTAGQEEYS

SEQ ID NO: 150 - protein, 11 aa, Artificial sequence
  synthetic peptide
QEEYSAMRDQY

SEQ ID NO: 151 - protein, 11 aa, Artificial sequence
  synthetic peptide
QEEYSAMRDQY

SEQ ID NO: 152 - protein, 11 aa, Artificial sequence
  synthetic peptide
QEEYSAMRDQY

SEQ ID NO: 153 - protein, 14 aa, Artificial sequence
  synthetic peptide
CALFGGTSYGKLTF

SEQ ID NO: 154 - protein, 16 aa, Artificial sequence
  synthetic peptide
CALSESGDAAGNKLTF

SEQ ID NO: 155 - protein, 13 aa, Artificial sequence
  synthetic peptide
CAEIPGGSYIPTF

SEQ ID NO: 156 - protein, 17 aa, Artificial sequence
  synthetic peptide
CASSLVSTPLPKETQYF

SEQ ID NO: 157 - protein, 15 aa, Artificial sequence
  synthetic peptide
CASSTPGPSAYEQYF

SEQ ID NO: 158 - protein, 11 aa, Artificial sequence
  synthetic peptide
CAEGENTEAFF

SEQ ID NO: 159 - protein, 14 aa, Artificial sequence
  synthetic peptide
CASSPWDIRTEAFF

SEQ ID NO: 160 - protein, 16 aa, Artificial sequence
  synthetic peptide
CALSESHNNAGNMLTF

SEQ ID NO: 161 - protein, 11 aa, Artificial sequence
  synthetic peptide
CAASQNTEAFF

SEQ ID NO: 162 - protein, 12 aa, Artificial sequence
  synthetic peptide
CIVRVPGGKLIF

SEQ ID NO: 163 - protein, 15 aa, Artificial sequence
  synthetic peptide
CAASDSGAGSYQLTF

SEQ ID NO: 164 - protein, 15 aa, Artificial sequence
  synthetic peptide
CLVGATFSGNTPLVF

SEQ ID NO: 165 - protein, 14 aa, Artificial sequence
  synthetic peptide
CALRDSNSGYALNF

SEQ ID NO: 166 - protein, 14 aa, Artificial sequence
  synthetic peptide
CAFRPPTGANNLFF

SEQ ID NO: 167 - protein, 14 aa, Artificial sequence
  synthetic peptide
CILRDGASYDKVIF

SEQ ID NO: 168 - protein, 13 aa, Artificial sequence
  synthetic peptide
CASLISDGQKLLF

SEQ ID NO: 169 - protein, 13 aa, Artificial sequence
  synthetic peptide
CAVSTDSSYKLIF

SEQ ID NO: 170 - protein, 11 aa, Artificial sequence
  synthetic peptide
CATDCKNQFYF

SEQ ID NO: 171 - protein, 16 aa, Artificial sequence
  synthetic peptide
CAASVSGGTSYGKLTF

SEQ ID NO: 172 - protein, 14 aa, Artificial sequence
  synthetic peptide
CAVRPEDNFNFKYF

SEQ ID NO: 173 - protein, 14 aa, Artificial sequence
  synthetic peptide
CAVPRGGSQGNLIF

SEQ ID NO: 174 - protein, 16 aa, Artificial sequence
  synthetic peptide
CASSLLEHRRGDTQYF

SEQ ID NO: 175 - protein, 18 aa, Artificial sequence
  synthetic peptide
CASSLGLIGPGMNTEAFF

SEQ ID NO: 176 - protein, 18 aa, Artificial sequence
  synthetic peptide
CASSPPSGRADETDTQYF

SEQ ID NO: 177 - protein, 14 aa, Artificial sequence
  synthetic peptide
CASSPWMGNQPQHF

SEQ ID NO: 178 - protein, 17 aa, Artificial sequence
  synthetic peptide
CASSQGGSRRLDYGYTF

SEQ ID NO: 179 - protein, 11 aa, Artificial sequence
  synthetic peptide
CASSQVEGVAF

SEQ ID NO: 180 - protein, 17 aa, Artificial sequence
  synthetic peptide
CASSLVSTPLPKETQYF

SEQ ID NO: 181 - protein, 18 aa, Artificial sequence
  synthetic peptide
CASSQGPVAGENTGELFF

SEQ ID NO: 182 - protein, 11 aa, Artificial sequence
  synthetic peptide
CASSPGTEAFF

SEQ ID NO: 183 - protein, 14 aa, Artificial sequence
  synthetic peptide
CASSFLATPDTQYF

SEQ ID NO: 184 - protein, 14 aa, Artificial sequence
  synthetic peptide
CSASGQHRRYGYTF

SEQ ID NO: 185 - protein, 11 aa, Artificial sequence
  synthetic peptide
CAGPQDYKLSF

SEQ ID NO: 186 - protein, 14 aa, Artificial sequence
  synthetic peptide
CAVRDRNNNARLMF

SEQ ID NO: 187 - protein, 13 aa, Artificial sequence
  synthetic peptide
CAVLTGGGNKLTF

SEQ ID NO: 188 - protein, 16 aa, Artificial sequence
  synthetic peptide
CALSESHNNAGNMLTF

SEQ ID NO: 189 - protein, 15 aa, Artificial sequence
  synthetic peptide
CAMRAASNTGNQFYF

SEQ ID NO: 190 - protein, 14 aa, Artificial sequence
  synthetic peptide
CAETPNSGNTPLVF

SEQ ID NO: 191 - protein, 16 aa, Artificial sequence
  synthetic peptide
CALSEPIYNQGGKLIF

SEQ ID NO: 192 - protein, 16 aa, Artificial sequence
  synthetic peptide
CALSDLSTSGTYKYIF

SEQ ID NO: 193 - protein, 16 aa, Artificial sequence
  synthetic peptide
CALSDLSTSGTYKYIF

SEQ ID NO: 194 - protein, 16 aa, Artificial sequence
  synthetic peptide
CASSPAPAGAFGEQYF

SEQ ID NO: 195 - protein, 17 aa, Artificial sequence
  synthetic peptide
CASSQQGQGEAGNTIYF

SEQ ID NO: 196 - protein, 11 aa, Artificial sequence
  synthetic peptide
CAASQNTEAFF

SEQ ID NO: 197 - protein, 16 aa, Artificial sequence
  synthetic peptide
CSARDTLRGYYNEQFF

SEQ ID NO: 198 - protein, 14 aa, Artificial sequence
  synthetic peptide
CASSLGVSNQPQHF

SEQ ID NO: 199 - protein, 11 aa, Artificial sequence
  Complementarity-determining region (CDR)  amino acid sequence
CATDCKNQFYF

SEQ ID NO: 200 - protein, 17 aa, Artificial sequence
  Complementarity-determining region (CDR)  amino acid sequence
CASSLVSTPLPKETQYF

SEQ ID NO: 201 - protein, 13 aa, Artificial sequence
  Complementarity-determining region (CDR)  amino acid sequence
CAESSGGGFKTIF

SEQ ID NO: 202 - protein, 15 aa, Artificial sequence
  Complementarity-determining region (CDR)  amino acid sequence
CASSTPGPSAYEQYF

SEQ ID NO: 203 - protein, 14 aa, Artificial sequence
  Complementarity-determining region (CDR)  amino acid sequence
CALFGGTSYGKLTF

SEQ ID NO: 204 - protein, 11 aa, Artificial sequence
  Complementarity-determining region (CDR)  amino acid sequence
CAEGENTEAFF

SEQ ID NO: 205 - protein, 16 aa, Artificial sequence
  Complementarity-determining region (CDR)  amino acid sequence
CALSESGDAAGNKLTF

SEQ ID NO: 206 - protein, 14 aa, Artificial sequence
  Complementarity-determining region (CDR)  amino acid sequence
CASSPWDIRTEAFF

SEQ ID NO: 207 - protein, 16 aa, Artificial sequence
  Complementarity-determining region (CDR)  amino acid sequence
CALSETINNAGNMLTF

SEQ ID NO: 208 - protein, 11 aa, Artificial sequence
  Complementarity-determining region (CDR)  amino acid sequence
CASSQNTEAFF

SEQ ID NO: 209 - protein, 16 aa, Artificial sequence
  Complementarity-determining region (CDR)  amino acid sequence
CALSESHNNAGNMLTF

SEQ ID NO: 210 - protein, 11 aa, Artificial sequence
  Complementarity-determining region (CDR)  amino acid sequence
CAASQNTEAFF

SEQ ID NO: 211 - protein, 33 aa, Artificial sequence
  Complementarity-determining region (CDR) nucleic acid sequence
TGTGCTACGGACTGTAAGAACCAGTTCTATTTT

SEQ ID NO: 212 - protein, 51 aa, Artificial sequence
  Complementarity-determining region (CDR) nucleic acid sequence
TGTGCCAGCAGTTTAGTGTCAACACCACTACCTAAAGAGACCCAGTACTTC

SEQ ID NO: 213 - protein, 39 aa, Artificial sequence
  Complementarity-determining region (CDR) nucleic acid sequence
TGTGCAGAGAGTAGCGGTGGAGGCTTCAAAACTATCTTT

SEQ ID NO: 214 - protein, 45 aa, Artificial sequence
  Complementarity-determining region (CDR) nucleic acid sequence
TGTGCCAGCAGTACACCGGGACCATCGGCGTACGAGCAGTACTTC

SEQ ID NO: 215 - protein, 42 aa, Artificial sequence
  Complementarity-determining region (CDR) nucleic acid sequence
TGTGCTCTATTTGGTGGTACTAGCTATGGAAAGCTGACATTT

SEQ ID NO: 216 - protein, 33 aa, Artificial sequence
  Complementarity-determining region (CDR) nucleic acid sequence
TGTGCCGAGGGAGAAAACACTGAAGCTTTCTTT

SEQ ID NO: 217 - protein, 48 aa, Artificial sequence
  Complementarity-determining region (CDR) nucleic acid sequence
TGTGCTCTGAGTGAGTCGGGGGATGCTGCAGGCAACAAGCTAACTTTT

SEQ ID NO: 218 - protein, 42 aa, Artificial sequence
  Complementarity-determining region (CDR) nucleic acid sequence
TGTGCCAGCAGTCCCTGGGACATTCGAACTGAAGCTTTCTTT

SEQ ID NO: 219 - protein, 48 aa, Artificial sequence
  Complementarity-determining region (CDR) nucleic acid sequence
TGTGCTCTGAGTGAGACCATTAATAATGCAGGCAACATGCTCACCTTT

SEQ ID NO: 220 - protein, 33 aa, Artificial sequence
  Complementarity-determining region (CDR) nucleic acid sequence
TGTGCCAGCAGCCAGAACACTGAAGCTTTCTTT

SEQ ID NO: 221 - protein, 48 aa, Artificial sequence
  Complementarity-determining region (CDR) nucleic acid sequence
TGTGCTCTGAGTGAGTCCCATAATAATGCAGGCAACATGCTCACCTTT

SEQ ID NO: 222 - protein, 33 aa, Artificial sequence
  Complementarity-determining region (CDR) nucleic acid sequence
TGTGCCGCGAGCCAGAACACTGAAGCTTTCTTT

SEQ ID NO: 223 - protein, 337 aa, Artificial sequence
  V region nucleotide sequence
ATGGAAACTCTCCTGGGAGTGTCTTTGGTGATTCTATGGCTTCAACTGGCTAGGGTGAACAGTCAACAGGGAGAAGAGGATCCTCAGGCCTTGAGCATCCAGGAGGGTGAAAATGCCACCATGAACTGCAGTTACAAAACTAGTATAAACAATTTACAGTGGTATAGACAAAATTCAGGTAGAGGCCTTGTCCACCTAATTTTAATACGTTCAAATGAAAGAGAGAAACACAGTGGAAGATTAAGAGTCACGCTTGACACTTCCAAGAAAAGCAGTTCCTTGTTGATCACGGCTTCCCGGGCAGCAGACACTGCTTCTTACTTCTGTGCTACGGACG

SEQ ID NO: 224 - protein, 344 aa, Artificial sequence
  V region nucleotide sequence
ATGGGCCCCCAGCTCCTTGGCTATGTGGTCCTTTGCCTTCTAGGAGCAGGCCCCCTGGAAGCCCAAGTGACCCAGAACCCAAGATACCTCATCACAGTGACTGGAAAGAAGTTAACAGTGACTTGTTCTCAGAATATGAACCATGAGTATATGTCCTGGTATCGACAAGACCCAGGGCTGGGCTTAAGGCAGATCTACTATTCAATGAATGTTGAGGTGACTGATAAGGGAGATGTTCCTGAAGGGTACAAAGTCTCTCGAAAAGAGAAGAGGAATTTCCCCCTGATCCTGGAGTCGCCCAGCCCCAACCAGACCTCTCTGTACTTCTGTGCCAGCAGTTTATC

SEQ ID NO: 225 - protein, 340 aa, Artificial sequence
  V region nucleotide sequence
ATGAAGACATTTGCTGGATTTTCGTTCCTGTTTTTGTGGCTGCAGCTGGACTGTATGAGTAGAGGAGAGGATGTGGAGCAGAGTCTTTTCCTGAGTGTCCGAGAGGGAGACAGCTCCGTTATAAACTGCACTTACACAGACAGCTCCTCCACCTACTTATACTGGTATAAGCAAGAACCTGGAGCAGGTCTCCAGTTGCTGACGTATATTTTTTCAAATATGGACATGAAACAAGACCAAAGACTCACTGTTCTATTGAATAAAAAGGATAAACATCTGTCTCTGCGCATTGCAGACACCCAGACTGGGGACTCAGCTATCTACTTCTGTGCAGAGAGTA

SEQ ID NO: 226 - protein, 344 aa, Artificial sequence
  V region nucleotide sequence
ATGAGCATCAGCCTCCTGTGCTGTGCAGCCTTTCCTCTCCTGTGGGCAGGTCCAGTGAATGCTGGTGTCACTCAGACCCCAAAATTCCGCATCCTGAAGATAGGACAGAGCATGACACTGCAGTGTACCCAGGATATGAACCATAACTACATGTACTGGTATCGACAAGACCCAGGCATGGGGCTGAAGCTGATTTATTATTCAGTTGGTGCTGGTATCACTGATAAAGGAGAAGTCCCGAATGGCTACAACGTCTCCAGATCAACCACAGAGGATTTCCCGCTCAGGCTGGAGTTGGCTGCTCCCTCCCAGACATCTGTGTACTTCTGTGCCAGCAGTTACTC

SEQ ID NO: 227 - protein, 329 aa, Artificial sequence
  V region nucleotide sequence
ATGAAGCCCACCCTCATCTCAGTGCTTGTGATAATATTTATACTCAGAGGAACAAGAGCCCAGAGAGTGACTCAGCCCGAGAAGCTCCTCTCTGTCTTTAAAGGGGCCCCAGTGGAGCTGAAGTGCAACTATTCCTATTCTGGGAGTCCTGAACTCTTCTGGTATGTCCAGTACTCCAGACAACGCCTCCAGTTACTCTTGAGACACATCTCTAGAGAGAGCATCAAAGGCTTCACTGCTGACCTTAACAAAGGCGAGACATCTTTCCACCTGAAGAAACCATTTGCTCAAGAGGAAGACTCAGCCATGTATTACTGTGCTCTAAGTGG

SEQ ID NO: 228 - protein, 344 aa, Artificial sequence
  V region nucleotide sequence
ATGAGCATCGGGCTCCTGTGCTGTGTGGCCTTTTCTCTCCTGTGGGCAAGTCCAGTGAATGCTGGTGTCACTCAGACCCCAAAATTCCAGGTCCTGAAGACAGGACAGAGCATGACACTGCAGTGTGCCCAGGATATGAACCATAACTCCATGTACTGGTATCGACAAGACCCAGGCATGGGACTGAGGCTGATTTATTACTCAGCTTCTGAGGGTACCACTGACAAAGGAGAAGTCCCCAATGGCTACAATGTCTCCAGATTAAACAAACGGGAGTTCTCGCTCAGGCTGGAGTCGGCTGCTCCCTCCCAGACATCTGTGTACTTCTGTGCCAGCAGTGAAGC

SEQ ID NO: 229 - protein, 350 aa, Artificial sequence
  V region nucleotide sequence
ATGCTGACTGCCAGCCTGTTGAGGGCAGTCATAGCCTCCATCTGTGTTGTATCCAGCATGGCTCAGAAGGTAACTCAAGCGCAGACTGAAATTTCTGTGGTGGAGAAGGAGGATGTGACCTTGGACTGTGTGTATGAAACCCGTGATACTACTTATTACTTATTCTGGTACAAGCAACCACCAAGTGGAGAATTGGTTTTCCTTATTCGTCGGAACTCTTTTGATGAGCAAAATGAAATAAGTGGTCGGTATTCTTGGAACTTCCAGAAATCCACCAGTTCCTTCAACTTCACCATCACAGCCTCACAAGTCGTGGACTCAGCAGTATACTTCTGTGCTCTGAGTGAGGC

SEQ ID NO: 230 - protein, 347 aa, Artificial sequence
  V region nucleotide sequence
ATGGACTCCTGGACCTTCTGCTGTGTGTCCCTTTGCATCCTGGTAGCGAAGCATACAGATGCTGGAGTTATCCAGTCACCCCGCCATGAGGTGACAGAGATGGGACAAGAAGTGACTCTGAGATGTAAACCAATTTCAGGCCACAACTCCCTTTTCTGGTACAGACAGACCATGATGCGGGGACTGGAGTTGCTCATTTACTTTAACAACAACGTTCCGATAGATGATTCAGGGATGCCCGAGGATCGATTCTCAGCTAAGATGCCTAATGCATCATTCTCCACTCTGAAGATCCAGCCCTCAGAACCCAGGGACTCAGCTGTGTACTTCTGTGCCAGCAGTTTAGC

SEQ ID NO: 231 - protein, 350 aa, Artificial sequence; identical to SEQ ID NO: 229
  V region nucleotide sequence

SEQ ID NO: 232 - protein, 344 aa, Artificial sequence; identical to SEQ ID NO: 226
  V region nucleotide sequence

SEQ ID NO: 233 - protein, 350 aa, Artificial sequence; identical to SEQ ID NO: 229
  V region nucleotide sequence

SEQ ID NO: 234 - protein, 344 aa, Artificial sequence; identical to SEQ ID NO: 228
  V region nucleotide sequence

SEQ ID NO: 235 - protein, 56 aa, Artificial sequence
  J region nucleotide sequence
GAACACCGGTAACCAGTTCTATTTTGGGACAGGGACAAGTTTGACGGTCATTCCAA

SEQ ID NO: 236 - protein, 48 aa, Artificial sequence
  J region nucleotide sequence
ACCAAGAGACCCAGTACTTCGGGCCAGGCACGCGGCTCCTGGTGCTCG

SEQ ID NO: 237 - protein, 61 aa, Artificial sequence
  J region nucleotide sequence
GGAAATACTGGAGGCTTCAAAACTATCTTTGGAGCAGGAACAAGACTATTTGTTAAAGCAA

SEQ ID NO: 238 - protein, 47 aa, Artificial sequence
  J region nucleotide sequence
CTCCTACGAGCAGTACTTCGGGCCGGGCACCAGGCTCACGGTCACAG

SEQ ID NO: 239 - protein, 69 aa, Artificial sequence
  J region nucleotide sequence
CTAATGCTGGTGGTACTAGCTATGGAAAGCTGACATTTGGACAAGGGACCATCTTGACTGTCCATCCAA

SEQ ID NO: 240 - protein, 48 aa, Artificial sequence
  J region nucleotide sequence
TGAACACTGAAGCTTTCTTTGGACAAGGCACCAGACTCACAGTTGTAG

SEQ ID NO: 241 - protein, 63 aa, Artificial sequence
  J region nucleotide sequence
TGATCAAAGCTGCAGGCAACAAGCTAACTTTTGGAGGAGGAACCAGGGTGCTAGTTAAACCAA

SEQ ID NO: 242 - protein, 48 aa, Artificial sequence; identical to SEQ ID NO: 240
  J region nucleotide sequence

SEQ ID NO: 243 - protein, 63 aa, Artificial sequence
  J region nucleotide sequence
TGAATAATAATGCAGGCAACATGCTCACCTTTGGAGGGGGAACAAGGTTAATGGTCAAACCCC

SEQ ID NO: 244 - protein, 48 aa, Artificial sequence; identical to SEQ ID NO: 240
  J region nucleotide sequence

SEQ ID NO: 245 - protein, 63 aa, Artificial sequence; identical to SEQ ID NO: 243
  J region nucleotide sequence

SEQ ID NO: 246 - protein, 48 aa, Artificial sequence; identical to SEQ ID NO: 240
  J region nucleotide sequence

SEQ ID NO: 247 - DNA, 31 bp, Artificial Sequence
  nucleic acid sequence surrounding mutation in NRAS
  misc_feature (16)..(16): n could be A or C
ctggatacagctgganaagaagagtacagtg